(19) United States
(12) Patent Application Publication
(10) Pub. No.: US 2010/0022627 A1
(43) Pub. Date: Jan. 28, 2010

(54) PREDICTIVE BIOMARKERS FOR CHRONIC ALLOGRAFT NEPHROPATHY

(76) Inventor: Andreas Scherer, Freiburg (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/295,298

(22) PCT Filed: Apr. 2, 2007
(86) PCT No.: PCT/EP2007/002953

§ 371 Date: Sep. 30, 2008

(30) Foreign Application Priority Data
Apr. 3, 2006 (GB) 0606776.3

(51) Int. Cl. A61K 31/7088; C12Q 1/68; G01N 33/53

(52) U.S. Cl. 514/44 R; 435/6; 435/7.1

(57) ABSTRACT

The invention relates to the analysis and identification of genes that are modulated in transplant rejection. This alteration of gene expression provides a molecular signature to accurately detect transplant rejection.

### FIELD OF THE INVENTION

This invention relates generally to the analytical testing of tissue samples in vitro, and more particularly to gene- or protein-based tests useful in prediction of chronic allograft nephropathy.

### BACKGROUND OF THE INVENTION

Chronic transplant dysfunction is a phenomenon in solid organ transplants displaying a gradual deterioration of graft function following transplantation, eventually leading to graft failure, and which is accompanied by characteristic histological features. Clinically, chronic transplant dysfunction in kidney grafts, e.g., chronic/sclerosing allograft nephropathy (“CAN”), manifests itself as a slowly progressive decline in glomerular filtration rate, usually in conjunction with proteinuria and arterial hypertension. Despite clinical application of potent immunoregulatory drugs and biologic agents, chronic rejection remains a common and serious post-transplantation complication. Chronic rejection is a relentlessly progressive process.

The single most common cause for early graft failure, especially within one month post-transplantation, is immunologic rejection of the allograft. The unfavorable impact of the rejection is magnified by the fact that: (a) the use of high-dose anti-rejection therapy, superimposed upon maintenance immunosuppression, is primarily responsible for the morbidity and mortality associated with transplantation, (b) the immunization against “public” HLA-specificities resulting from a rejected graft renders this patient population difficult to retransplant and (c) the return of the immunized recipient with a failed graft to the pool of patients awaiting transplantation enhances the perennial problem of organ shortage.

Histopathological evaluation of biopsy tissue is the gold standard for the diagnosis of CAN, while prediction of the onset of CAN is currently impossible. Current monitoring and diagnostic modalities are ill-suited to the diagnosis of CAN at an early stage.

### SUMMARY

The invention pertains to molecular diagnostic methods using gene expression profiling further refine the BANFF 97 disease classification (Racusen L C, et al., Kidney Int. 55(2):713-23 (1999)). The invention also provides for methods for using biomarkers as predictive or early diagnostic biomarkers when applied at early time points after transplantation when graft dysfunction by other more conventional means is not yet detectable.

Accordingly, in one aspect, the invention pertains to a method for predicting the onset of a rejection of a transplanted organ in a subject, comprising the steps of: (a) obtaining a post-transplantation sample from the subject; (b) determining the level of gene expression in the post-transplantation sample of a combination of a plurality of genes selected from the group consisting of the genes of: Table 4; Table 5; Table 6; Table 7; and Table 8 in combination with a predictive model selected from the group consisting of a PLDSA model and an OPLS model; (c) comparing the magnitude of gene expression of the at least one gene in the post-transplantation sample with the magnitude of gene expression of the same gene in a control sample; and (d) determining whether the expression level of at least one gene is up-regulated or down-regulated relative to the control sample, wherein up-regulation or down-regulation of at least one gene indicates that the subject is likely to experience transplant rejection, thereby predicting the onset of rejection of the transplanted organ in the subject.

The sample comprises cells obtained from the subject. The sample can be selected from the group consisting of: a graft biopsy; blood; serum; and urine. The rejection can be chronic/sclerosing allograph nephropathy. The magnitude of expression in the sample differs from the control magnitude of expression by a factor of at least about 1.5, or by a factor of at least about 2.

In another aspect, the invention pertains to a method for predicting the onset of a rejection of a transplanted organ in a subject, comprising the steps of: (a) obtaining a post-transplantation sample from the subject; (b) determining the level of gene expression in the post-transplantation sample of a combination of a plurality of genes selected from the group consisting of the genes of: Table 4; Table 5; Table 6; Table 7; and Table 8 in combination with a predictive model selected from the group consisting of a PLDSA model and an OPLS model; and (c) comparing the gene expression pattern of the combination of gene in the post-transplantation sample with the pattern of gene expression of the same combination of gene in a control sample, wherein a similarity in the expression pattern of the gene expression pattern of the combination of gene in the post-transplantation sample compared to the expression pattern same combination of gene in a control sample expression profile indicates indicates that the subject is likely to experience transplant rejection, thereby predicting the onset of rejection of the transplanted organ in the subject.

In another aspect, the invention pertains to a method of monitoring transplant rejection in a subject, comprising the steps of: (a) taking as a baseline value the magnitude of gene expression of a combination of a plurality of genes in a sample obtained from a transplanted subject who is known not to develop rejection; (b) detecting a magnitude of gene expression corresponding to the combination of a plurality of genes in a sample obtained from a patient post-transplantation; and (c) comparing the first value with the second value, wherein a first value lower or higher than the second value predicts that the transplanted subject is at risk of developing rejection, wherein the plurality of genes are selected from the group consisting of the genes of: Table 4; Table 5; Table 6; Table 7; and Table 8 in combination with a predictive model selected from the group consisting of a PLDSA model and an OPLS model.

In another aspect, the invention pertains to a method of monitoring transplant rejection in a subject, comprising the steps of: (a) detecting a pattern of gene expression corresponding to a combination of a plurality of genes from a sample obtained from a donor subject at the day of transplantation; (b) detecting a pattern of gene expression corresponding to the plurality of genes from a sample obtained from a recipient subject post-transplantation; and (c) comparing the first value with the second value, wherein a first value lower or higher than the second value predicts that the recipient subject is at risk of developing rejection; wherein the a plurality of genes selected from the group consisting of the genes of: Table 4; Table 5; Table 6; Table 7; and Table 8 in combination with a predictive model selected from the group consisting of a PLDSA model and an OPLS model.

In another aspect, the invention pertains to a method for monitoring transplant rejection in a subject at risk thereof, comprising the steps of: (a) obtaining a pre-administration sample from a transplanted subject prior to administration of a rejection inhibiting agent; (b) detecting the magnitude of gene expression of a plurality of genes in the pre-administration sample; and (c) obtaining one or more post-administration samples from the transplanted subject; detecting the pattern of gene expression of a plurality of genes in the post-administration sample or samples, comparing the pattern of gene expression of the plurality of genes in the pre-administration sample with the pattern of gene expression in the post-administration sample or samples, and adjusting the agent accordingly, wherein the plurality of genes are selected from the group consisting of the genes of: Table 2; Table 3 and Table 4 in combination with a predictive model selected from the group consisting of a PLDSA model and an OPLS model.

In another aspect, the invention pertains to a method for preventing, inhibiting, reducing or treating transplant rejection in a subject in need of such treatment comprising administering to the subject a compound that modulates the synthesis, expression or activity of one or more genes or gene products encoded thereof of genes selected from the group consisting of the genes of: Table 4; Table 5; Table 6; Table 7; and Table 8 in combination with a predictive model selected from the group consisting of a PLDSA model and an OPLS model, so that at least one symptom of rejection is ameliorated.

In another aspect, the invention pertains to a method for identifying agents for use in the prevention, inhibition, reduction or treatment of transplant rejection comprising monitoring the level of gene expression of one or more genes or gene products selected from the group consisting of the genes of: Table 4; Table 5; Table 6; Table 7; and Table 8 in combination with a predictive model selected from the group consisting of a PLDSA model and an OPLS model.

The transplanted subject can be a kidney transplanted subject. The pattern of gene expression can be assessed by detecting the presence of a protein encoded by the gene. The presence of the protein can be detected using a reagent which specifically binds to the protein. The pattern of gene expression can be detected by techniques selected from the group consisting of Northern blot analysis, reverse transcription PCR and real time quantitative PCR. The magnitude of gene expression of one gene or a plurality of genes can be detected.

In another aspect, the invention pertains to use of the combination of the plurality of genes or an expression products thereof as listed in Table 2, Table 3 or Table 4 in combination with a predictive model selected from the group consisting of a PLDSA model and an OPLS model as a biomarker for transplant rejection.

In another aspect, the invention pertains to use of a compound which modulates the synthesis, expression of activity of one or more genes as identified in Table 2, Table 3 or Table 4 in combination with a predictive model selected from the group consisting of a PLDSA model and an OPLS model, or an expression product thereof, for the preparation of a medicament for prevention or treatment of transplant rejection in a subject.

### BRIEF DESCRIPTION OF DRAWINGS

FIG. 1 is a schematic diagram detailing the time course of biopsy samples for diagnosis of stable allograft function (normal, N) and chronic allograft rejection (CAN) by histopathological evaluation;

FIG. 2 is a scatter plot derived by partial least squares discrimination analysis (PLDA) of biomarker data obtained at Biomarker week 06;

FIG. 3 is a graph derived by PLSDA of data obtained at Biomarker week 06 comparing observed versus predicted biomarker data;

FIG. 4 is a graph of biomarker data relating to the Biomarker week 06 PLSDA model: Validation by Response Permutation;

FIG. 5 is a scatter plot derived by orthogonal partial least squares analysis (OPLS) of biomarker data obtained at Biomarker week 12;

FIG. 6 is a graph of biomarker data relating to the Biomarker week 12 OPLS model: Validation by Response Permutation;

FIG. 7 is a graph derived by OPLS of data obtained at Biomarker week 12 comparing observed versus predicted biomarker data;

FIG. 8 is a scatter plot derived by PLDA of biomarker data obtained at Biomarker week 06;

FIG. 9 is a graph of biomarker data relating to the Biomarker week 12 PLSDA model: Validation by Response Permutation;

FIG. 10 is a graph derived by OPLS of data obtained at Biomarker week 12 comparing observed versus predicted biomarker data;

FIG. 11 is a scatter plot derived by orthogonal signal correction (OSC) in a global analysis of biomarker data;

FIG. 12 is a graph of biomarker data relating to Biomarker global analysis OSC model: Validation by response permutation;

FIG. 13 is a graph derived by global analysis OSC modeling of data comparing observed versus predicted biomarker data;

FIG. 14 is a scatter plot derived by OPLS in a global analysis of biomarker data; and

FIG. 15 is a graph derived by global analysis OPLS modeling of data comparing observed versus predicted biomarker data.

FIG. 16 is a chart showing week 6 post-TX timepoint, 4.5 months before clinical/histopath. evidence of CAN.

FIG. 17 is graph of biomarker identification at week 6 (4.5 months before CAN). Good separation of patient groups (PLSDA model with 49 probe sets).

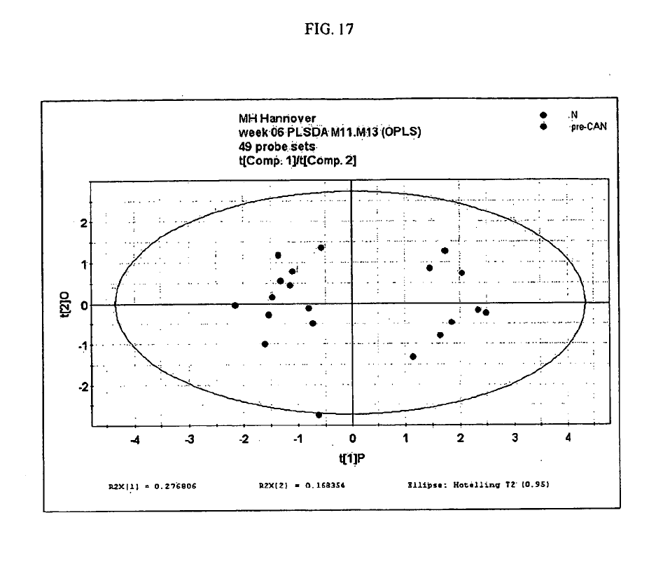

FIG. 18 is graph showing cross-validation at week 6 (4.5 months before CAN). Cross-validation (“leave one group of 7 samples out”): Model provides clear separation between N and pre-CAN.

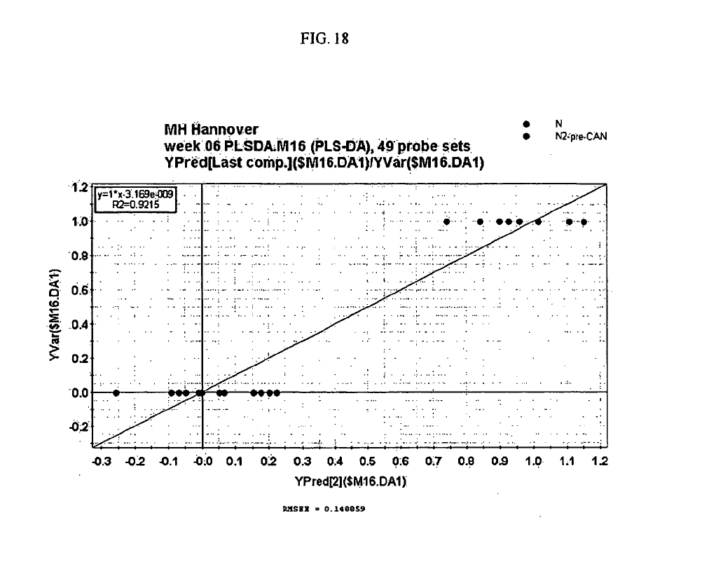

FIG. 19 is a chart showing week 6 post-TX timepoint, 3 months before clinical/histopath. evidence of CAN.

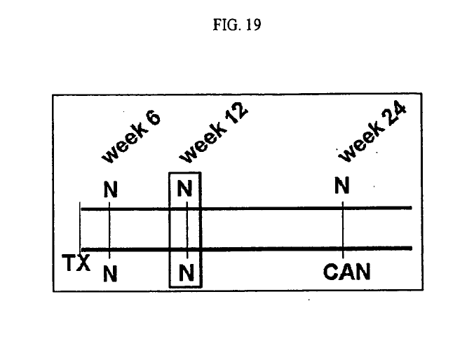

FIG. 20 is a chart showing the overlap of biomarkers identified at week 6 (t test<0.05, 1.2 FC) and week 12 (t test<0.05, 1.5 FC). Small overlap between week 06 and week 12 biological genelists may indicate the presence of different underlying biological processes/pathways at specific timepoints.

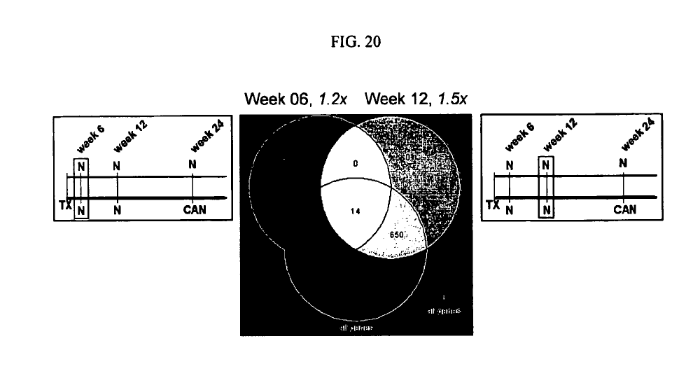

FIG. 21 is a figure the OSC model with 201 probe sets. OSC model with 201 probe sets differentiates groups by timepoint and diagnosis.

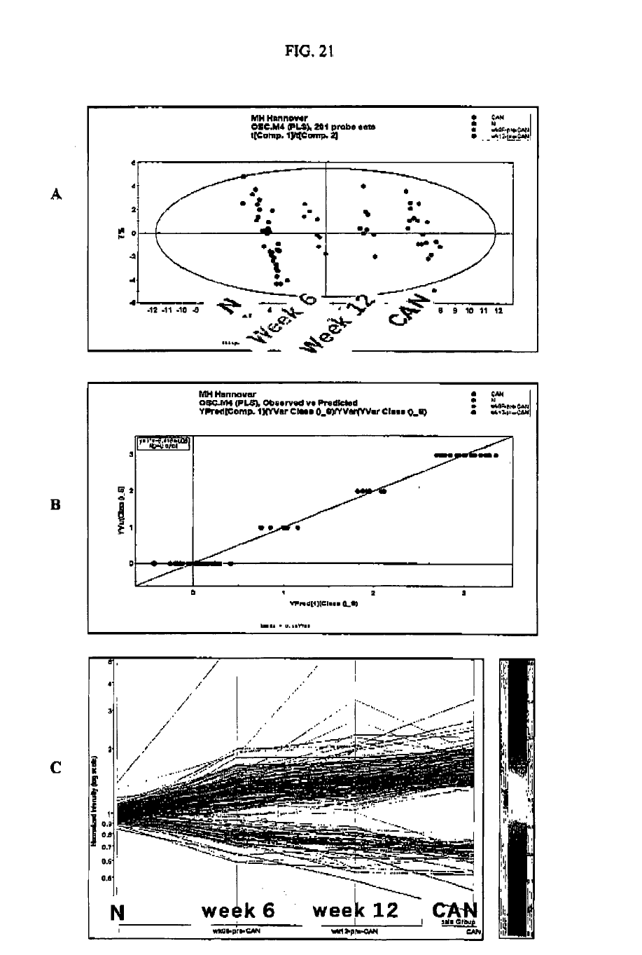

FIG. 22 is a figure showing pathway analysis and biological mechanisms. Transient activation of pathways at different timepoints.

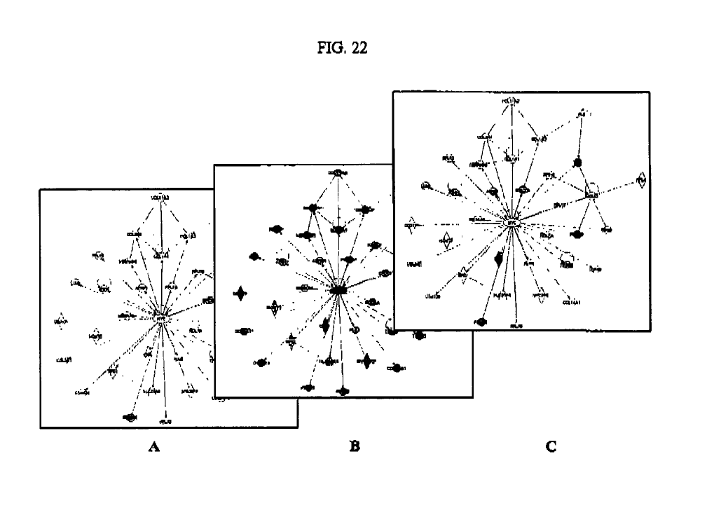

FIG. 23 is a figure showing model validation by permutation. Model validation by Permutation analysis: 100 iterations (i.e. fit of 100 PLS models compared to fit of“real model”).

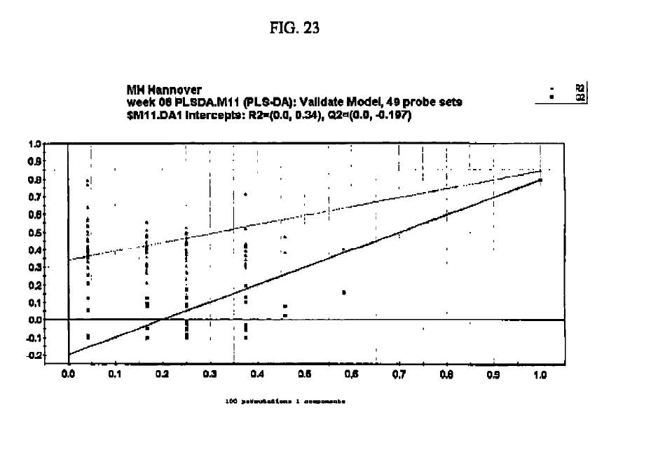

### DETAILED DESCRIPTION

Definitions

To further facilitate an understanding of the present invention, a number of terms and phrases are defined below:

The terms “down-regulation” or “down-regulated” are used interchangeably herein and refer to the decrease in the amount of a target gene or a target protein. The term “down-regulation” or “down-regulated” also refers to the decreases in processes or signal transduction cascades involving a target gene or a target protein.

The term “transplantation” as used herein refers to the process of taking a cell, tissue, or organ, called a “transplant” or “graft” from one subject and placing it or them into a (usually) different subject. The subject who provides the transplant is called the “donor” and the subject who received the transplant is called the “recipient”. An organ, or graft, transplanted between two genetically different subjects of the same species is called an “allograft”. A graft transplanted between subjects of different species is called a “xenograft”.

The term “transplant rejection” as used herein is defined as functional and structural deterioration of the organ due to an active immune response expressed by the recipient, and independent of non-immunologic causes of organ dysfunction.

The term “chronic rejection” as used herein refers to rejection of the transplanted organs (e.g., kidney). The term also applies to a process leading to loss of graft function and late graft loss developing after the first 30-120 post-transplant days. In kidneys, the development of nephrosclerosis (hardening of the renal vessels), with proliferation of the vascular intima of renal vessels, and intimal fibrosis, with marked decrease in the lumen of the vessels, takes place. The result is renal ischemia, hypertension, tubular atrophy, interstitial fibrosis, and glomerular atrophy with eventual renal failure. In addition to the established influence of HLA incompatibility, the age, number of nephrons, and ischemic history of a donor kidney may contribute to ultimate progressive renal failure in transplanted patients.

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

A “gene” includes a polynucleotide containing at least one open reading frame that is capable of encoding a particular polypeptide or protein after being transcribed and translated. Any of the polynucleotide sequences described herein may be used to identify larger fragments or full-length coding sequences of the gene with which they are associated. Methods of isolating larger fragment sequences are known to those of skill in the art, some of which are described herein.

A “gene product” includes an amino acid (e.g., peptide or polypeptide) generated when a gene is transcribed and translated.

The term “magnitude of expression” as used herein refers to quantifying marker gene transcripts and comparing this quantity to the quantity of transcripts of a constitutively expressed gene. The term “magnitude of expression” means a “normalized, or standardized amount of gene expression”. For example, the overall expression of all genes in cells varies (i.e., it is not constant). To accurately assess whether the detection of increased mRNA transcript is significant, it is preferable to “normalize” gene expression to accurately compare levels of expression between samples, i.e., it is a baselevel against which gene expression is compared. In one embodiment, the expressed gene is associated with a biological pathway/process selected from the group consisting of: the wnt pathway (e.g., NFAT, NE-dig, frizzled-9, hes-1), TGFbeta (e.g., NOMO, SnoN), glucose and fatty acid transport and metabolism (e.g., GLUT4), vascular smooth muscle differentiation (e.g., amnionless, ACLP, lumican), vascular sclerosis (e.g., THRA, IGFBP4), ECM (e.g., collagen), and immune response (e.g., TNF, NFAT, GM-CSF). Quantification of gene transcripts was accomplished using competitive reverse transcription polymerase chain reaction (RT-PCR) and the magnitude of gene expression was determined by calculating the ratio of the quantity of gene expression of each marker gene to the quantity of gene expression of the expressed gene.

The term “differentially expressed”, as applied to a gene, includes the differential production of mRNA transcribed from a gene or a protein product encoded by the gene. A differentially expressed gene may be overexpressed or underexpressed as compared to the expression level of a normal or control cell. In one aspect, it includes a differential that is at least 2 times, at least 3 times, at least 4 times, at least 5 times, at least 6 times, at least 7 times, at least 8 times, at least 9 times or at least 10 times higher or lower than the expression level detected in a control sample. In a preferred embodiment, the expression is higher than the control sample. The term “differentially expressed” also includes nucleotide sequences in a cell or tissue which are expressed where silent in a control cell or not expressed where expressed in a control cell. In particular, this term refers to refers to a given allograft gene expression level and is defined as an amount which is substantially greater or less than the amount of the corresponding baseline expression level. Baseline is defined here as being the level of expression in healthy tissue. Healthy tissue includes a transplanted organ without pathological findings.

The term “sample” as used herein refers to cells obtained from a biopsy. The term “sample” also refers to cells obtained from a fluid sample including, but not limited to, a sample of bronchoalveolar lavage fluid, a sample of bile, pleural fluid or peritoneal fluid, or any other fluid secreted or excreted by a normally or abnormally functioning allograft, or any other fluid resulting from exudation or transudation through an allograft or in anatomic proximity to an allograft, or any fluid in fluid communication with the allograft. A fluid test sample may also be obtained from essentially any body fluid including: blood (including peripheral blood), lymphatic fluid, sweat, peritoneal fluid, pleural fluid, bronchoalveolar lavage fluid, pericardial fluid, gastrointestinal juice, bile, urine, feces, tissue fluid or swelling fluid, joint fluid, cerebrospinal fluid, or any other named or unnamed fluid gathered from the anatomic area in proximity to the allograft or gathered from a fluid conduit in fluid communication with the allograft. A “post-transplantation fluid test sample” refers to a sample obtained from a subject after the transplantation has been performed.

Sequential samples can also be obtained from the subject and the quantification of immune activation gene biomarkers determined as described herein, and the course of rejection can be followed over a period of time. In this case, for example, the baseline magnitude of gene expression of the biomarker gene(s) is the magnitude of gene expression in a post-transplant sample taken after the transplant. For example, an initial sample or samples can be taken within the nonrejection period, for example, within one week of transplantation and the magnitude of expression of biomarker genes in these samples can be compared with the magnitude of expression of the genes in samples taken after one week. In one embodiment, the samples are taken on weeks 6, 12 and 24 post-transplantation.

The term “biopsy” as used herein refers to a specimen obtained by removing tissue from living patients for diagnostic examination. The term includes aspiration biopsies, brush biopsies, chorionic villus biopsies, endoscopic biopsies, excision biopsies, needle biopsies (specimens obtained by removal by aspiration through an appropriate needle or trocar that pierces the skin, or the external surface of an organ, and into the underlying tissue to be examined), open biopsies, punch biopsies (trephine), shave biopsies, sponge biopsies, and wedge biopsies. In one embodiment, a fine needle aspiration biopsy is used. In another embodiment, a minicore needle biopsy is used. A conventional percutaneous core needle biopsy can also be used.

The term “up-regulation” or “up-regulated” are used interchangeably herein and refer to the increase or elevation in the amount of a target gene or a target protein. The term “up-regulation” or “up-regulated” also refers to the increase or elevation of processes or signal transduction cascades involving a target gene or a target protein.

The term “gene cluster” or “cluster” as used herein refers to a group of genes related by expression pattern. In other words, a cluster of genes is a group of genes with similar regulation across different conditions, such as graft non-rejection versus graft rejection. The expression profile for each gene in a cluster should be correlated with the expression profile of at least one other gene in that cluster. Correlation may be evaluated using a variety of statistical methods. Often, but not always, members of a gene cluster have similar biological functions in addition to similar gene expression patterns.

A “probe set” as used herein refers to a group of nucleic acids that may be used to detect two or more genes. Detection may be, for example, through amplification as in PCR and RT-PCR, or through hybridization, as on a microarray, or through selective destruction and protection, as in assays based on the selective enzymatic degradation of single or double stranded nucleic acids. Probes in a probe set may be labeled with one or more fluorescent, radioactive or other detectable moieties (including enzymes). Probes may be any size so long as the probe is sufficiently large to selectively detect the desired gene. A probe set may be in solution, as would be typical for multiplex PCR, or a probe set may be adhered to a solid surface, as in an array or microarray. It is well known that compounds such as PNAs may be used instead of nucleic acids to hybridize to genes. In addition, probes may contain rare or unnatural nucleic acids such as inosine.

The terms “polynucleotide” and “oligonucleotide” are used interchangeably, and include polymeric forms of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for guanine when the polynucleotide is RNA. This, the term “polynucleotide sequence” is the alphabetical representation of a polynucleotide molecule. This alphabetical representation can be inputted into databases in a computer having a central processing unit and used for bioinformatics applications such as functional genomics and homology searching.

The term “cDNAs” includes complementary DNA, that is mRNA molecules present in a cell or organism made into cDNA with an enzyme such as reverse transcriptase. A “cDNA library” includes a collection of mRNA molecules present in a cell or organism, converted into cDNA molecules with the enzyme reverse transcriptase, then inserted into “vectors” (other DNA molecules that can continue to replicate after addition of foreign DNA). Exemplary vectors for libraries include bacteriophage, viruses that infect bacteria (e g., lambda phage). The library can then be probed for the specific cDNA (and thus mRNA) of interest.

A “primer” includes a short polynucleotide, generally with a free 3′-OH group that binds to a target or “template” present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or “set of primers” consisting of “upstream” and a “downstream” primer, and a catalyst of polymerization, such as a DNA polymerase, and typically a thermally-stable polymerase enzyme. Methods for PCR are well known in the art, and are taught, for example, in MacPherson et al., IRL Press at Oxford University Press (1991)). All processes of producing replicate copies of a polynucleotide, such as PCR or gene cloning, are collectively referred to herein as “replication”. A primer can also be used as a probe in hybridization reactions, such as Southern or Northern blot analyses (see, e.g., Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

The term “polypeptide” includes a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” includes either natural and/or unnatural or synthetic amino acids, including glycine and both the D or L optical isomers, and amino acid analogs and peptidomimetics. A peptide of three or more amino acids is commonly referred to as an oligopeptide. Peptide chains of greater than three or more amino acids are referred to as a polypeptide or a protein.

The term “hybridization” includes a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. The stringency of a hybridization reaction includes the difficulty with which any two nucleic acid molecules will hybridize to one another. Under stringent conditions, nucleic acid molecules at least 60%, 65%, 70%, 75% identical to each other remain hybridized to each other, whereas molecules with low percent identity cannot remain hybridized. A preferred, non-limiting example of highly stringent hybridization conditions are hybridization in 6x sodium chloride/sodium citrate (SSC) at about 45° C., followed by one or more washes in 0.2×SSC, 0.1% SDS at 50° C., preferably at 55° C., more preferably at 60° C., and even more preferably at 65° C.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to hydrogen bond with each other, according to generally accepted base-pairing rules.

As used herein, the terms “marker” and “biomarker” are used interchangeably and include a polynucleotide or polypeptide molecule which is present or modulated (i.e., increased or decreased) in quantity or activity determined using a statistical model (e.g., PLSDA and OPLS), in subjects at risk for organ rejection relative to the quantity or activity in subjects that are not at risk for organ rejection. The relative change in quantity or activity of the biomarker is correlated with the incidence or risk of incidence of rejection.

As used herein, the term “panel of markers” includes a group of biomarkers determined using a statistical model (e.g., PLSDA and OPLS), the quantity or activity of each member of which is correlated with the incidence or risk of incidence of organ rejection. In certain embodiments, a panel of biomarkers may include only those biomarkers which are either increased in quantity or activity in subjects at risk for organ rejection. In other embodiments, a panel of biomarkers may include only those biomarkers which are either decreased in quantity or activity in subjects at risk for organ rejection.

Abbreviations for select terms are summarized in Table 1 below.

TABLE 1
Abbreviations:

| Abbreviation | Term |
|---|---|
| AEBP/ACLP | Adipocyte enhancer binding protein/aortic |
| | carboxylase like protein |
| Amn | amnionless |
| BMD | BioMarker Development |
| CAN | Chronic allograft nephropathy |
| CP | Ceruloplasmin, ferroxidase |
| CSF2RB | colony stimulating factor 2 receptor, beta |
| CV | Coefficient of variance |
| Dlg3, Ne-dlg | Neuroendocrine discs large |
| Fzd-9 | Frizzled 9 |
| GLUT4/ | solute carrier family 2 (facilitated glucose |
| SLC2A12 | transporter), member 12 |
| Hes-1 | Hairy and enhancer of split 1 |
| HGF | hepatocyte growth factor (hepapoietin A; |
| | scatter factor) |
| IGFBP4 | insulin-like growth factor binding protein 4 |
| Lcn | lumican |
| NFAT | Nuclear factor of activated T cells |
| OPLS | Orthogonal projections of latent structures by |
| | means of partial least squares |
| PLS | Projections of latent structures by means of |
| | partial least squares |
| PLS-DA | Projections of latent structures by means of |
| | partial least squares-discriminant analysis |
| pM5/NOMO | Nodal modulator 2 |
| Ski-l/SnoN | Ski-like (snoN) |
| THRA | Thyroid hormone receptor alpha |

Predictive Biomarkers of Chronic Rejection

The invention is based, in part, on the discovery that select genes are modulated in CAN and these genes can be used as predictive biomarkers before the onset of overt CAN. Advances in highly parallel, automated DNA hybridization techniques combined with the growing wealth of human gene sequence information have made it feasible to simultaneously analyze expression levels for thousands of genes (see, e.g., Schena et al., 1995, Science 270:467-470; Lockhart et al., 1996, Nature Biotechnology 14:1675-1680; Blanchard et al., 1996, Nature Biotechnology 14:1649; Ashby et al., U.S. Pat. No. 5,569,588, issued Oct. 29, 1996; Perou et al., 2000, Nature 406:747-752). Methods such as the gene-by-gene quantitative RT-PCR are highly accurate but relatively labor intensive. While it is possible to analyze the expression of thousands of genes using quantitative PCR, the effort and expense would be enormous. Instead, as an example of large scale analysis, an entire population of mRNAs may be converted to cDNA and hybridized to an ordered array of probes that represent anywhere from ten to ten thousand or more genes. The relative amount of cDNA that hybridizes to each of these probes is a measure of the expression level of the corresponding gene. The data may then be statistically analyzed to reveal informative patterns of gene expression. Indeed, early diagnosis of renal allograft rejection and new prognostic biomarkers are important minimize and personalize immunosuppression. In addition to histopathological differential diagnosis, gene expression profiling significantly improves disease classification by defining a “molecular signature.”

Several previous studies have successfully applied a transcriptomic approach to distinguish different classes of kidney transplants. However, the heterogeneity of microarray platforms and various data analysis methods complicates the identification of robust signatures of CAN.

To address this issue, comparative multivariate data analyses (e.g., PLSDA; OPLS; OSC) was performed on gene expression profiles of serial renal protocol biopsies from patients with stable graft function throughout at least one year after renal transplantation and patients who had diagnosed chronic allograft nephropathy (CAN; grade 1) at the week 24 biopsy but not at biopsies of earlier time points (week 06 and week 12). As presented in Example I, these studies identify molecular signatures predictive of the onset of CAN. The molecular signature comprises a combination of algorithm and genes identified by the algorithm at various time points. That is, the present invention relates to the identification of genes, which are modulated (i.e., up-regulated or down-regulated) during rejection, in particular during early CAN. A highly statistically significant correlation has been found between the expression of one or more biomarker gene(s) and CAN, thereby providing a “molecular signature” for transplant rejection (e.g., CAN). These biomarker genes and their expression products can be used in the management, prognosis and treatment of patients at risk of transplant rejection as they are useful to identify organs that are likely to undergo rejection.

Clinical Features of CAN

Chronic transplant dysfunction is a phenomenon in solid organ transplants displaying a gradual deterioration of graft function months to years after transplantation, eventually leading to graft failure, and which is accompanied by characteristic histological features. Clinically, chronic allograft nephropathy in kidney grafts (i.e., CAN) manifests itself as a slowly progressive decline in glomerular filtration rate, usually in conjunction with proteinuria and arterial hypertension.

The cardinal histomorphologic feature of CAN in all parenchymal allografts is fibroproliferative endarteritis. The vascular lesion affects the whole length of the arteries in a patchy pattern. There is concentric myointimal proliferation resulting in fibrous thickening and the characteristic ‘onion skin’ appearance of the intima in small arteries. Other findings include endothelial swelling, foam cell accumulation, disruption of the internal elastic lamina, hyalinosis and medial thickening, and presence of subendothelial T-lymphocytes and macrophages (Hruban R H, et al., Am J Pathol 137(4):871-82 (1990)). In addition, a persistent focal perivascular inflammation is often seen.

In addition to vascular changes, kidneys undergoing CAN also show interstitial fibrosis, tubular atrophy, and glumerulopathy. Chronic transplant glumerolopathy—duplication of the capillary walls and mesangial matrix increase—has been identified as a highly specific feature of kidneys with CAN (Solez K, Clin Transplant.; 8(3 Pt 2):345-50 (1994)). Less specific lesions are glomerular ischemic collapse, tubular atrophy, and interstitial fibrosis. Furthermore, peritubular capillary basement splitting and laminations are associated with late decline of graft function (Monga M, et al., Ultrastruct Pathol. 14(3):201-9 (1990)). The criteria for histological diagnosis of CAN in kidney allografts are internationally standardized in the Banff 97 scheme for Renal Allograft Pathology (Racusen L C, et al., Kidney Int. 55(2):713-23 (1999)); (adopted from Kouwenhoven et al., Transpl Int. 2000;13(6):385-401. 2000). Table 2 summarizes the Banff 97 criteria for chronic/sclerosing allograft nephropathy (CAN) (Racusen L C, et al., Kidney Int. 55(2):713-23 (1999)).

TABLE 2

| Grade | Histopathological Findings |
|---|---|
| I - mild | Mild interstitial fibrosis and tubular atrophy without (a) |
| | or with (b) specific changes suggesting chronic rejection |
| II - moderate | Moderate interstitial fibrosis and tubular atrophy (a) or (b) |
| III - severe | Severe interstitial fibrosis and tubular atrophy and |
| | tubular loss (a) or (b) |

For Banff 97, an “adequate” specimen is defined as a biopsy with 10 or more glumeruli and at least two arteries. Two working hypotheses are proposed to understand the process of CAN (Kouwenhoven et al., Transpl Int. 2000;13(6):385-401. 2000). The first and probably the most important set of risk factors have been lumped under the designation of “alloantigen-dependent”, immunological or rejection-related factors. Among these, late onset and increased number of acute rejection episodes; younger recipient age; male-to-female sex mismatch; a primary diagnosis of autoimmune hepatitis or biliary disease; baseline immunosuppression and non-caucasian recipient race have all been associated with an increased risk of developing chronic rejection. More specifically, (a) histoincompatibility: long-term graft survival appear to be strongly correlated with their degree of histocompatibility matching between donor and recipient; (b) Acute rejections: onset, frequency, and severity of acute rejection episodes are independent risk factors of CAN. Acute rejection is the most consistently identified risk factor for the occurrence of CAN; (c) Suboptimal immunosuppression due to too low maintenance dose of cyclosporine or non-compliance; and (d) Anti-donor specific antibodies: many studies have shown that following transplantation, the majority of patients produce antibodies. The second set of risk factors are referred to as “non-alloantigen-dependent” or “non-immunological” risk factors that also contribute to the development of chronic rejection include advanced donor age, pre-existing atherosclerosis in the donor organ, and prolonged cold ischemic time. Non-alloimmune responses to disease and injury, such as ischemia, can cause or aggravate CAN. More specifically, (a) recurrence of the original disease, such as glomerulonephritis; (b) consequence of the transplantation surgical injury; (c) duration of ischemia: intimal hyperplasia correlates with duration of ischemia; (d) kidney grafts from cadavers versus those from living related and unrelated donors; (e) viral infections: CMV infection directly affects intercellular adhesion molecules such as ICAM-1; (f) hyperlipidemia; (g) hypertension; (h) age; (i) gender: the onset of transplant arterosclerosis was earlier in male than in female; (j) race; and (k) the amount of functional tissue—reduced number of nephrons and hyperfiltration.

CAN is characterized by morphological evidence of destruction of the transplanted organ. The common denominator of all parenchymal organs is the development of intimal hyperplasia. T cells and macrophages are the predominant graft-invading cell types, with an excess of CD4⁺ over CD8⁺ T cells. Increased expression of adhesion molecules (ICAM-1, VCAM-1) and MHC antigens are seen in allografts with CAN, and increased TGF-β is frequently found. A short description of the route through which a graft may develop CAN follows:

Endothelial Cell Activation by Ischemia, Surgical Manipulation, and Reperfusion Injury.

In consequence, the endothelial cells produce oxygen free radicals and they release increased amounts of the cytokines IL-1, IL-6, IFN-γ, TNF-α and the chemokines IL-8, macrophage chemoattractant protein 1 (MCP-1), macrophage inflammatory protein 1α and 1β (MIP-1α MIP-1 β), colony stimulating factors, and multiple growth factors such as, platelet derived growth factor (PDGF), insulin like growth factor 1 (IGF-1), transforming growth factor β (TGF-β), and pro-thrombotic molecules such as tissue factor and plasminogen activator inhibitor (PAI). These cytokines activate the migration of neutrophils, monocytes/macrophages and T-lymphocytes to the site of injury where they interact with the endothelial cells by means of adhesion molecules, including ICAM-1, VCAM-1, P- and E-selectin. The increased expression of these adhesion molecules is induced by the cytokines IL-1β, IFN-γ, and TNF-α. Extravasation of leucocytes is facilitated by activated complement and oxygen-free radicals that increase the permeability between endothelial cells.

Limitations to Current Clinical Approaches for CAN Diagnosis

The differentiation of the diagnosis of rejection, e.g., CAN, from other etiologies for graft dysfunction and institution of effective therapy is a complex process because: (a) the percutaneous core needle biopsy of grafts, the best of available current tools to diagnose rejection is performed usually after the “fact”, i.e., graft dysfunction and graft damage (irreversible in some instances) are already present, (b) the morphological analysis of the graft provides modest clues with respect to the potential for reversal of a given rejection episode, and minimal clues regarding the likelihood of recurrence (“rebound”), and (c) the mechanistic basis of the rejection phenomenon, a prerequisite for the design of therapeutic strategies, is poorly defined by current diagnostic indices, including morphologic features of rejection.

The diagnosis of, for example, renal allograft rejection is made usually by the development of graft dysfunction (e.g., an increase in the concentration of serum creatinine) and morphologic evidence of graft injury in areas of the graft also manifesting mononuclear cell infiltration. Two caveats apply, however, to the use of abnormal renal function as an indicator of the rejection process: first, deterioration in renal function is not always available as a clinical clue to diagnose rejection since many of the cadaveric renal grafts suffer from acute (reversible) renal failure in the immediate post-transplantation period due to injury from harvesting and ex vivo preservation procedures. Second, even when immediately unimpaired renal function is present, graft dysfunction might develop due to a non-immunologic cause, such as immunosuppressive therapy itself.

For example, cyclosporine (CsA) nephrotoxicity, a complication that is not readily identified solely on the basis of plasma/blood concentrations of CsA, is a common complication. The clinical importance of distinguishing rejection from CsA nephrotoxicity cannot be overemphasized since the therapeutic strategies are diametrically opposite: escalation of immunosuppressants for rejection, and reduction of CsA dosage for nephrotoxicity.

The invention is based, in part, on the observation that increased or decreased expression of on or more genes and/or the encoded proteins is associated with certain graft rejection states. As a result of the data described herein, methods are now available for the rapid and reliable diagnosis of acute and chronic rejection, even in cases where allograft biopsies show only mild cellular infiltrates. Described herein is an analysis of genes that are modulated (e.g., up-regulated or down-regulated) simultaneously and which provide a molecular signature to accurately detect transplant rejection.

The invention further provides classic molecular methods and large scale methods for measuring expression of suitable biomarker genes. The methods described herein are particularly useful for detecting chronic transplant rejection and preferably early chronic transplant rejection. In one embodiment, the chronic transplant rejection is the result of CAN. Most typically, the subject (i.e., the recipient of a transplant) is a mammal, such as a human. The transplanted organ can include any transplantable organ or tissue, for example kidney, heart, lung, liver, pancreas, bone, bone marrow, bowel, nerve, stem cells (or stem cell-derived cells), tissue component and tissue composite. In a preferred embodiment, the transplant is a kidney transplant.

The methods described herein are useful to assess the efficacy of anti-rejection therapy. Such methods involve comparing the pre-administration magnitude of the transcripts of the biomarker genes to the post-administration magnitude of the transcripts of the same genes, where a post-administration magnitude of the transcripts of the genes that is less than the pre-administration magnitude of the transcripts of the same genes indicates the efficacy of the anti-rejection therapy. Any candidates for prevention and/or treatment of transplant rejection, (such as drugs, antibodies, or other forms of rejection or prevention) can be screened by comparison of magnitude of biomarker expression before and after exposure to the candidate. In addition, valuable information can be gathered in this manner to aid in the determination of future clinical management of the subject upon whose biological material the assessment is being performed. The assessment can be performed using a sample from the subject, using the methods described herein for determining the magnitude of gene expression of the biomarker genes. Analysis can further comprise detection of an infectious agent.

Biological Pathways Associated with Biomarkers of the Invention

Biomarkers of the present invention identify select biological pathways affected by CAN and, as such, these biological pathways are of relevance to solid organ allograft nephropathy. Indeed, this meta-analysis revealed robust biomarker signatures for select biological pathways which can represent gene clusters. Such biological pathways include, but are not limited to, e.g., wnt pathway (i.e., NFAT (Murphy et al., J Immunol. 69(7):3717-25 (2002)); NE-dlg (Hanada et al., Int. J. Cancer 86(4):480-8 (2000)); frizzled-9 (Karasawa et al., J. Biol. Chem. 277(40):37479-86 (2002)); Hes-1 (Deregowski et al., J Biol Chem. 281(10):6203-10 (2006); Piscione et al., Gene Expr. Patterns 4(6):707-11 (2004)), TGFbeta/Smad signaling pathway (i.e., Smad3 (Saika et al., Am. J. Pathol. 164(2):651-63 (2004); Smad2 (Ju et al., Mol. Cell Biol. 26(2):654-67 (2006); pM5/NOMO (Hafner et al., EMBO J. Aug. 4, 2004;23(15):3041-50; SnoN (Zhu et al., Mol. Cell Biol. 25(24):10731-44 (2005); Wilkinson et al., Mol. Cell Biol. 25(3):1200-12 (2005)), glucose and fatty acid transport and metabolism (i.e., GLUT4 (Linden et al., Am J Physiol Renal Physiol. 290(1):F205-13. (2006)), vascular smooth muscle differentiation (i.e., lumican (Onda et al., 72(2): 142-9 (2002); ceruloplasmin (Chen et al., Biochem. Biophys. Res. Commun. 281(2):475-82 (2001); amnionless (Moestrup S K, Curr Opin Lipidol. 16(3):301-6 (2005); aortic carboxypeptidase-like protein (ACLP)), vascular sclerosis (THRA (Sato et al, Circ. Res. 97(6):550-7 (2005); IGFBP4; AE binding protein-1 (Layne et al., J. Biol. Chem. 273(25):15654-60 (1998); Abderrahim et al, Exp. Cell Res. 293(2):219-28 (2004)); ECM (collagen), and immune response (NFAT (Murphy et al., J Immunol. 69(7):3717-25 (2002));TNF, GM-CSF (Steinman R. M., Annu Rev. Immunol 9:271-96 (1991); Xu et al., Trends Pharmacol. Sci. 25(5):254-8 (2004)). Jehle and coworkers have demonstrated that insulin-like growth factor binding protein 4 in serum is characteristic of chronic renal failure. Jehle et al., Kidney Int. 57(3):1209-10 (2000). Azuma and coworkers have shown that Hepatocyte growth factor (HGF) plays a renotropic role in renal regeneration and protection from acute ischemic injury and that HGF treatment greatly contribute to a reduction of susceptibility to the subsequent development of CAN in a rat model. Azuma et al. J. Am. Soc. Nephrol. 12(6):1280-92 (2001).

The advent of large scale gene expression analysis has revealed that groups of genes are often expressed together in a coordinated manner. For example, whole genome expression analysis in the yeast Saccharomyces cerevisiae showed coordinate regulation of metabolic genes during a change in growth conditions known as the diauxic shift (DiRisi et al., 1997, Science 278:680-686; Eisen et al., 1998, PNAS 95:14863-14868). The diauxic shift occurs when yeast cells fermenting glucose to ethanol exhaust the glucose in the media and begin to metabolize the ethanol. In the presence of glucose, genes of the glycolytic pathway are expressed and carry out the fermentation of glucose to ethanol. When the glucose is exhausted, yeast cells must metabolize the ethanol, a process that depends heavily on the Krebs cycle and respiration.

Accordingly, the expression of glycolysis genes decreases, and the expression of Krebs cycle and respiratory genes increases in a coordinate manner. Similar coordinate gene regulation has been found in various cancer cells. Genes encoding proteins involved in cell cycle progression and DNA synthesis are often coordinately overexpressed in cancerous cells (Ross et al., 2000, Nature Genet. 24:227-235; Perou et al, 1999, PNAS 96:9212-9217; Perou et al., 2000, Nature 406:747-752).

The coordinate regulation of genes is logical from a functional point of view. Most cellular processes require multiple genes, for example: glycolysis, the Krebs cycle, and cell cycle progression are all multi-gene processes. Coordinate expression of functionally related genes is therefore essential to permit cells to perform various cellular activities. Such groupings of genes can be called “gene clusters” (Eisen et al., 1998, PNAS 95:14863-68).

Clustering of gene expression is not only a functional necessity, but also a natural consequence of the mechanisms of transcriptional control. Gene expression is regulated primarily by transcriptional regulators that bind to cis-acting DNA sequences, also called regulatory elements. The pattern of expression for a particular gene is the result of the sum of the activities of the various transcriptional regulators that act on that gene. Therefore, genes that have a similar set of regulatory elements will also have a similar expression pattern and will tend to cluster together. Of course, it is also possible, and quite common, for genes that have different regulatory elements to be expressed coordinately under certain circumstances.

It is anticipated that the analysis of more than one gene cluster will be useful not only for diagnosing transplant rejection but also for determining appropriate medical interventions. For example, chronic allograft nephropathy is a general description for a disorder that has many variations and many different optimal treatment strategies. In one embodiment, the invention provides a method for simultaneously identifying graft rejection and determining an appropriate treatment. In general, the invention provides methods comprising measuring representatives of different, informative biomarker genes which can represent gene clusters, that indicate an appropriate treatment protocol.

Detecting Gene Expression

In certain aspects of the present invention, the magnitude of expression is determined for one or more biomarker genes in sample obtained from a subject. The sample can comprise cells obtained from the subject, such as from a graft biopsy. Other samples include, but are not limited to fluid samples such as blood, plasma, serum, lymph, CSF, cystic fluid, ascites, urine, stool and bile. The sample may also be obtained from bronchoalveolar lavage fluid, pleural fluid or peritoneal fluid, or any other fluid secreted or excreted by a normally or abnormally functioning allograft, or any other fluid resulting from exudation or transudation through an allograft or in anatomic proximity to an allograft, or any fluid in fluid communication with the allograft.

Many different methods are known in the art for measuring gene expression. Classical methods include quantitative RT-PCR, Northern blots and ribonuclease protection assays. Certain examples described herein use competitive reverse transcription (RT)-PCR to measure the magnitude of expression of biomarker genes. Such methods may be used to examine expression of subject genes as well as entire gene clusters. However, as the number of genes to be examined increases, the time and expense may become cumbersome.

Large scale detection methods allow faster, less expensive analysis of the expression levels of many genes simultaneously. Such methods typically involve an ordered array of probes affixed to a solid substrate. Each probe is capable of hybridizing to a different set of nucleic acids. In one method, probes are generated by amplifying or synthesizing a substantial portion of the coding regions of various genes of interest. These genes are then spotted onto a solid support. Then, mRNA samples are obtained, converted to cDNA, amplified and labeled (usually with a fluorescence label). The labeled cDNAs are then applied to the array, and cDNAs hybridize to their respective probes in a manner that is linearly related to their concentration. Detection of the label allows measurement of the amount of each cDNA adhered to the array. Many methods for performing such DNA array experiments are well known in the art. Exemplary methods are described below but are not intended to be limiting.

Microarrays are known in the art and consist of a surface to which probes that correspond in sequence to gene products (e.g., cDNAs, mRNAs, oligonucleotides) are bound at known positions. In one embodiment, the microarray is an array (i.e., a matrix) in which each position represents a discrete binding site for a product encoded by a gene (e.g., a protein or RNA), and in which binding sites are present for products of most or almost all of the genes in the organism's genome. In a preferred embodiment, the “binding site” (hereinafter, “site”) is a nucleic acid or nucleic acid derivative to which a particular cognate cDNA can specifically hybridize. The nucleic acid or derivative of the binding site can be, e.g., a synthetic oligomer, a full-length cDNA, a less-than full length cDNA, or a gene fragment.

Usually the microarray will have binding sites corresponding to at least 100 genes and more preferably, 500, 1000, 4000 or more. In certain embodiments, the most preferred arrays will have about 98-100% of the genes of a particular organism represented. In other embodiments, customized microarrays that have binding sites corresponding to fewer, specifically selected genes can be used. In certain embodiments, customized microarrays comprise binding sites for fewer than 4000, fewer than 1000, fewer than 200 or fewer than 50 genes, and comprise binding sites for at least 2, preferably at least 3, 4, 5 or more genes of any of the biomarkers of Table 4, Table 5, Table 6, Table 7, and Table 8. Preferably, the microarray has binding sites for genes relevant to testing and confirming a biological network model of interest.

The nucleic acids to be contacted with the microarray may be prepared in a variety of ways. Methods for preparing total and poly(A)+ RNA are well known and are described generally in Sambrook et al., supra. Labeled cDNA is prepared from mRNA by oligo dT-primed or random-primed reverse transcription, both of which are well known in the art (see e.g., Klug and Berger, 1987, Methods Enzymol. 152:316-325). Reverse transcription may be carried out in the presence of a dNTP conjugated to a detectable label, most preferably a fluorescently labeled dNTP. Alternatively, isolated mRNA can be converted to labeled antisense RNA synthesized by in vitro transcription of double-stranded cDNA in the presence of labeled dNTPs (Lockhart et al., 1996, Nature Biotech. 14:1675). The cDNAs or RNAs can be synthesized in the absence of detectable label and may be labeled subsequently, e.g., by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent.

When fluorescent labels are used, many suitable fluorophores are known, including fluorescein, lissamine, phycoerythrin, rhodamine (Perkin Elmer Cetus), Cy2, Cy3, Cy3.5, Cy5, Cy5.5, Cy7, FluorX (Amersham) and others (see, e.g., Kricka, 1992, Academic Press San Diego, Calif.).

In another embodiment, a label other than a fluorescent label is used. For example, a radioactive label, or a pair of radioactive labels with distinct emission spectra, can be used (see Zhao et al., 1995, Gene 156:207; Pietu et al., 1996, Genome Res. 6:492). However, use of radioisotopes is a less-preferred embodiment.

Nucleic acid hybridization and wash conditions are chosen so that the population of labeled nucleic acids will specifically hybridize to appropriate, complementary nucleic acids affixed to the matrix. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch.

Optimal hybridization conditions will depend on the length (e.g., oligomer versus polynucleotide greater than 200 bases) and type (e.g., RNA, DNA, PNA) of labeled nucleic acids and immobilized polynucleotide or oligonucleotide. General parameters for specific (i.e., stringent) hybridization conditions for nucleic acids are described in Sambrook et al., supra, and in Ausubel et al., 1987, Current Protocols in Molecular Biology, Greene Publishing and Wiley-Interscience, New York, which is incorporated in its entirety for all purposes. Non-specific binding of the labeled nucleic acids to the array can be decreased by treating the array with a large quantity of non-specific DNA—a so-called “blocking” step.

When fluorescently labeled probes are used, the fluorescence emissions at each site of a transcript array can be, preferably, detected by scanning confocal laser microscopy. When two fluorophores are used, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al., 1996, Genome Research 6:639-645). In a preferred embodiment, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., 1996, Genome Res. 6:639-645 and in other references cited herein. Alternatively, the fiber-optic bundle described by Ferguson et al., 1996, Nature Biotech. 14:1681-1684, may be used to monitor mRNA abundance levels at a large number of sites simultaneously. Fluorescent microarray scanners are commercially available from Affymetrix, Packard BioChip Technologies, BioRobotics and many other suppliers.

Signals are recorded, quantitated and analyzed using a variety of computer software. In one embodiment the scanned image is despeckled using a graphics program (e.g. Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores is preferably calculated. The ratio is independent of the absolute expression level of the cognate gene, but is useful for genes whose expression is significantly modulated by drug administration, gene deletion, or any other tested event.

In one embodiment, transcript arrays reflecting the transcriptional state of a cell of interest are made by hybridizing a mixture of two differently labeled sets of cDNAs to the microarray. One cell is a cell of interest while the other is used as a standardizing control. The relative hybridization of each cell's cDNA to the microarray then reflects the relative expression of each gene in the two cells.

In preferred embodiments, expression levels of genes of a biomarker model in different samples and conditions may be compared using a variety of statistical methods. A variety of statistical methods are available to assess the degree of relatedness in expression patterns of different genes. The statistical methods may be broken into two related portions: metrics for determining the relatedness of the expression pattern of one or more gene, and clustering methods, for organizing and classifying expression data based on a suitable metric (Sherlock, 2000, Curr. Opin. Immunol. 12:201-205; Butte et al., 2000, Pacific Symposium on Biocomputing, Hawaii, World Scientific, p. 418-29).

In one embodiment, Pearson correlation may be used as a metric. In brief, for a given gene, each data point of gene expression level defines a vector describing the deviation of the gene expression from the overall mean of gene expression level for that gene across all conditions. Each gene's expression pattern can then be viewed as a series of positive and negative vectors. A Pearson correlation coefficient can then be calculated by comparing the vectors of each gene to each other. An example of such a method is described in Eisen et al. (1998, supra). Pearson correlation coefficients account for the direction of the vectors, but not the magnitudes.

In another embodiment, Euclidean distance measurements may be used as a metric. In these methods, vectors are calculated for each gene in each condition and compared on the basis of the absolute distance in multidimensional space between the points described by the vectors for the gene. In another embodiment, both Euclidean distance and Correlation coefficient were used in the clustering.

In a further embodiment, the relatedness of gene expression patterns may be determined by entropic calculations (Butte et al. 2000, supra). Entropy is calculated for each gene's expression pattern. The calculated entropy for two genes is then compared to determine the mutual information. Mutual information is calculated by subtracting the entropy of the joint gene expression patterns from the entropy calculated for each gene individually. The more different two gene expression patterns are, the higher the joint entropy will be and the lower the calculated mutual information. Therefore, high mutual information indicates a non-random relatedness between the two expression patterns.

In another embodiment, agglomerative clustering methods may be used to identify gene clusters. In one embodiment, Pearson correlation coefficients or Euclidean metrics are determined for each gene and then used as a basis for forming a dendrogram. In one example, genes were scanned for pairs of genes with the closest correlation coefficient. These genes are then placed on two branches of a dendrogram connected by a node, with the distance between the depth of the branches proportional to the degree of correlation. This process continues, progressively adding branches to the tree. Ultimately a tree is formed in which genes connected by short branches represent clusters, while genes connected by longer branches represent genes that are not clustered together. The points in multidimensional space by Euclidean metrics may also be used to generate dendrograms.

In yet another embodiment, divisive clustering methods may be used. For example, vectors are assigned to each gene's expression pattern, and two random vectors are generated. Each gene is then assigned to one of the two random vectors on the basis of probability of matching that vector. The random vectors are iteratively recalculated to generate two centroids that split the genes into two groups. This split forms the major branch at the bottom of a dendrogram. Each group is then further split in the same manner, ultimately yielding a fully branched dendrogram.

In a further embodiment, self-organizing maps (SOM) may be used to generate clusters. In general, the gene expression patterns are plotted in n-dimensional space, using a metric such as the Euclidean metrics described above. A grid of centroids is then placed onto the n-dimensional space and the centroids are allowed to migrate towards clusters of points, representing clusters of gene expression. Finally the centroids represent a gene expression pattern that is a sort of average of a gene cluster. In certain embodiments, SOM may be used to generate centroids, and the genes clustered at each centroid may be further represented by a dendrogram. An exemplary method is described in Tamayo et al, 1999, PNAS 96:2907-12 Once centroids are formed, correlation must be evaluated by one of the methods described supra.

In another embodiment, PLSDA, OPLS and OSC multivariate analyses may be used as a means of classification. As detailed in Example I, the biomarker models of the invention (e.g., PLSDA, OPLS and OSC models and the genes identified by such models) are useful to classify tissue with latent CAN and/or early CAN.

In another aspect, the invention provides probe sets. Preferred probe sets are designed to detect expression of one or more genes and provide information about the status of a graft. Preferred probe sets of the invention comprise probes that are useful for the detection of at least two genes belonging to any of the biomarker genes of Table 4, Table 5, Table 6, Table 7, and Table 8. Probe sets of the invention comprise probes useful for the detection of no more than 10,000 gene transcripts, and preferred probe sets will comprise probes useful for the detection of fewer than 4000, fewer than 1000, fewer than 200, fewer than 100, fewer than 90, fewer than 80, fewer than 70, fewer than 60, fewer than 50, fewer than 40, fewer than 30, fewer than 20, fewer than 10 gene transcripts. The probe sets of the invention are targeted at the detection of gene transcripts that are informative about transplant status. Probe sets of the invention may also comprise a large or small number of probes that detect gene transcripts that are not informative about transplant status. In preferred embodiments, probe sets of the invention are affixed to a solid substrate to form an array of probes. It is anticipated that probe sets may also be useful for multiplex PCR. The probes of probe sets may be nucleic acids (e.g., DNA, RNA, chemically modified forms of DNA and RNA), or PNA, or any other polymeric compound capable of specifically interacting with the desired nucleic acid sequences.

Computer readable media comprising a biomarker(s) of the present invention is also provided. As used herein, “computer readable media” includes a medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. The skilled artisan will readily appreciate how any of the presently known computer readable mediums can be used to create a manufacture comprising computer readable medium having recorded thereon a biomarker of the present invention.

As used herein, “recorded” includes a process for storing information on computer readable medium. Those skilled in the art can readily adopt any of the presently known methods for recording information on computer readable medium to generate manufactures comprising the biomarkers of the present invention.

A variety of data processor programs and formats can be used to store the biomarker information of the present invention on computer readable medium. For example, the nucleic acid sequence corresponding to the biomarkers can be represented in a word processing text file, formatted in commercially-available software such as WordPerfect and MicroSoft Word, or represented in the form of an ASCII file, stored in a database application, such as DB2, Sybase, Oracle, or the like. Any number of dataprocessor structuring formats (e.g., text file or database) may be adapted in order to obtain computer readable medium having recorded thereon the biomarkers of the present invention.

By providing the biomarkers of the invention in computer readable form, one can routinely access the biomarker sequence information for a variety of purposes. For example, one skilled in the art can use the nucleotide or amino acid sequences of the invention in computer-readable form to compare a target sequence or target structural motif with the sequence information stored within the data storage means. Search means are used to identify fragments or regions of the sequences of the invention which match a particular target sequence or target motif.

The invention also includes an array comprising a biomarker(s) of the present invention. The array can be used to assay expression of one or more genes in the array. In one embodiment, the array can be used to assay gene expression in a tissue to ascertain tissue specificity of genes in the array. In this manner, up to about 4700 genes can be simultaneously assayed for expression. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development and differentiation, disease progression, in vitro processes, such a cellular transformation and senescence, autonomic neural and neurological processes, such as, for example, pain and appetite, and cognitive functions, such as learning or memory.

The array is also useful for ascertaining the effect of the expression of a gene on the expression of other genes in the same cell or in different cells. This provides, for example, for a selection of alternate molecular targets for therapeutic intervention if the ultimate or downstream target cannot be regulated.

The array is also useful for ascertaining differential expression patterns of one or more genes in normal and diseased cells. This provides a battery of genes that could serve as a molecular target for diagnosis or therapeutic intervention.

Proteins

It is further anticipated that increased levels of certain proteins may also provide diagnostic information about transplants. In certain embodiments, one or more proteins encoded by genes of Table 4, Table 5, Table 6, Table 7, and Table 8 may be detected, and elevated or decreased protein levels may be used to predict graft rejection. In a preferred embodiment, protein levels are detected in a post-transplant fluid sample, and in a particularly preferred embodiment, the fluid sample is peripheral blood or urine. In another preferred embodiment, protein levels are detected in a graft biopsy.

In view of this specification, methods for detecting proteins are well known in the art. Examples of such methods include Western blotting, enzyme-linked immunosorbent assays (ELISAs), one- and two-dimensional electrophoresis, mass spectroscopy and detection of enzymatic activity. Suitable antibodies may include polyclonal, monoclonal, fragments (such as Fab fragments), single chain antibodies and other forms of specific binding molecules.

Predictive Medicine

The present invention pertains to the field of predictive medicine in which diagnostic assays, prognostic assays, pharmacogenetics and monitoring clinical trials are used for prognostic (predictive) purposes to thereby diagnose and treat a subject prophylactically. Accordingly, one aspect of the present invention relates to diagnostic assays for determining biomarker protein and/or nucleic acid expression from a sample (e.g., blood, serum, cells, tissue) to thereby determine whether a subject is likely to reject a transplant.

Another aspect of the invention pertains to monitoring the influence of agents (e.g., drugs, compounds) on the expression or activity of biomarker in clinical trials as described in further detail in the following sections.

An exemplary method for detecting the presence or absence of biomarker protein or genes of the invention in a sample involves obtaining a sample from a test subject and contacting the sample with a compound or an agent capable of detecting the protein or nucleic acid (e.g., mRNA, genomic DNA) that encodes the biomarker protein such that the presence of the biomarker protein or nucleic acid is detected in the sample. A preferred agent for detecting mRNA or genomic DNA corresponding to a biomarker gene or protein of the invention is a labeled nucleic acid probe capable of hybridizing to a mRNA or genomic DNA of the invention. Suitable probes for use in the diagnostic assays of the invention are described herein.

A preferred agent for detecting biomarker protein is an antibody capable of binding to biomarker protein, preferably an antibody with a detectable label. Antibodies can be polyclonal, or more preferably, monoclonal. An intact antibody, or a fragment thereof (eg., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “sample” is intended to include tissues, cells and biological fluids isolated from a subject, as well as tissues, cells and fluids present within a subject. That is, the detection method of the invention can be used to detect biomarker mRNA, protein, or genomic DNA in a sample in vitro as well as in vivo. For example, in vitro techniques for detection of biomarker mRNA include Northern hybridizations and in situ hybridizations. In vitro techniques for detection of biomarker protein include enzyme linked immunosorbent assays (ELISAs), Western blots, immunoprecipitations and immunofluorescence. In vitro techniques for detection of biomarker genomic DNA include Southern hybridizations. Furthermore, in vivo techniques for detection of biomarker protein include introducing, into a subject, a labeled anti-biomarker antibody. For example, the antibody can be labeled with a radioactive biomarker whose presence and location in a subject can be detected by standard imaging techniques.

In one embodiment, the sample contains protein molecules from the test subject. Alternatively, the sample can contain mRNA molecules from the test subject or genomic DNA molecules from the test subject. A preferred sample is a serum sample isolated by conventional means from a subject.

The methods further involve obtaining a control sample (e.g., biopsies from non transplanted healthy kidney or from transplanted healthy kidney showing no sign of rejection) from a control subject, contacting the control sample with a compound or agent capable of detecting biomarker protein, mRNA, or genomic DNA, such that the presence of biomarker protein, mRNA or genomic DNA is detected in the sample, and comparing the presence of biomarker protein, mRNA or genomic DNA in the control sample with the presence of biomarker protein, mRNA or genomic DNA in the test sample.

The invention also encompasses kits for detecting the presence of biomarker in a sample. For example, the kit can comprise a labeled compound or agent capable of detecting biomarker protein or mRNA in a sample; means for determining the amount of biomarker in the sample; and means for comparing the amount of biomarker in the sample with a standard. The compound or agent can be packaged in a suitable container. The kit can further comprise instructions for using the kit to detect biomarker protein or nucleic acid.

The diagnostic methods described herein can furthermore be utilized to identify subjects having or at risk of developing a disease or disorder associated with aberrant biomarker expression or activity. As used herein, the term “aberrant” includes a biomarker expression or activity which deviates from the wild type biomarker expression or activity. Aberrant expression or activity includes increased or decreased expression or activity, as well as expression or activity which does not follow the wild type developmental pattern of expression or the subcellular pattern of expression. For example, aberrant biomarker expression or activity is intended to include the cases in which a mutation in the biomarker gene causes the biomarker gene to be under-expressed or over-expressed and situations in which such mutations result in a non-functional biomarker protein or a protein which does not function in a wild-type fashion, e.g., a protein which does not interact with a biomarker ligand or one which interacts with a non-biomarker protein ligand.

Furthermore, the prognostic assays described herein can be used to determine whether a subject can be administered an agent (e.g., an agonist, antagonist, peptidomimetic, protein, peptide, nucleic acid, small molecule, or other drug candidate) to reduce the risk of rejection, e.g., cyclospsorin. Thus, the present invention provides methods for determining whether a subject can be effectively treated with an agent for a disorder associated with increased gene expression or activity of the combination of genes in Table 4, Table 5, Table 6, Table 7, and Table 8.

Monitoring the influence of agents (e.g., drugs) on the expression or activity of a genes can be applied not only in basic drug screening, but also in clinical trials. For example, the effectiveness of an agent determined by a screening assay as described herein to increase gene expression, protein levels, or up-regulate activity, can be monitored in clinical trials of subjects exhibiting by examining the molecular signature and any changes in the molecular signature during treatment with an agent.

For example, and not by way of limitation, genes and their encoded proteins that are modulated in cells by treatment with an agent (e.g., compound, drug or small molecule) which modulates gene activity can be identified. In a clinical trial, cells can be isolated and RNA prepared and analyzed for the levels of expression of genes implicated associated with rejection. The levels of gene expression (e.g., a gene expression pattern) can be quantified by northern blot analysis or RT-PCR, as described herein, or alternatively by measuring the amount of protein produced, by one of the methods as described herein. In this way, the gene expression pattern can serve as a molecular signature, indicative of the physiological response of the cells to the agent. Accordingly, this response state may be determined before, and at various points during treatment of the subject with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a gene or combination of genes, the protein encoded by the genes, mRNA, or genomic DNA in the preadministration sample; (iii) obtaining one or more post-administration samples from the subject; (iv) detecting the level of expression or activity of the biomarker protein, mRNA, or genomic DNA in the post-administration samples; (v) comparing the level of expression or activity of the biomarker protein, mRNA, or genomic DNA in the pre-administration sample with the a gene or combination of genes, the protein encoded by the genes, mRNA, or genomic DNA in the post administration sample or samples; and (vi) altering the administration of the agent to the subject accordingly. For example, increased administration of the agent may be desirable to decrease the expression or activity of the genes to lower levels, i.e., to increase the effectiveness of the agent to protect against transplant rejection. Alternatively, decreased administration of the agent may be desirable to decrease expression or activity of biomarker to lower levels than detected, i.e., to decrease the effectiveness of the agent e.g., to avoid toxicity. According to such an embodiment, gene expression or activity may be used as an indicator of the effectiveness of an agent, even in the absence of an observable phenotypic response.

The present invention provides for both prophylactic and therapeutic methods for preventing transplant rejection. With regards to both prophylactic and therapeutic methods of treatment, such treatments may be specifically tailored or modified, based on knowledge obtained from the field of pharmacogenomics. “Pharmacogenomics”, as used herein, includes the application of genomics technologies such as gene sequencing, statistical genetics, and gene expression analysis to drugs in clinical development and on the market. More specifically, the term refers the study of how a subject's genes determine his or her response to a drug (e.g., a subject's “drug response phenotype”, or “drug response genotype”). Thus, another aspect of the invention provides methods for tailoring a subject's prophylactic or therapeutic treatment with either the biomarker molecules of the present invention or biomarker modulators according to that subject's drug response genotype. Pharmacogenomics allows a clinician or physician to target prophylactic or therapeutic treatments to subjects who will most benefit from the treatment and to avoid treatment of subjects who will experience toxic drug-related side effects.

In one aspect, the invention provides a method for preventing transplant rejection in a subject, associated with increased biomarker expression or activity, by administering to the subject a compound or agent which modulates biomarker expression. Examples of such compounds or agents are e.g., compounds or agents having immunosuppressive properties, such as those used in transplantation (e.g., a calcineurin inhibitor, cyclosporin A or FK 506); a mTOR inhibitor (e.g., rapamycin, 40-O -(2-hydroxyethyl)-rapamycin, CC1779, ABT578, AP23573, biolimus-7 or biolimus-9); an ascomycin having immuno-suppressive properties (e.g., ABT-281, ASM981, etc.); corticosteroids; cyclophosphamide; azathioprene; methotrexate; leflunomide; mizoribine; mycophenolic acid or salt; mycophenolate mofetil; 15-deoxyspergualine or an immunosuppressive homologue, analogue or derivative thereof; a PKC inhibitor (e.g., as disclosed in WO 02/38561 or WO 03/82859, the compound of Example 56 or 70); a JAK3 kinase inhibitor (e.g., N-benzyl-3,4dihydroxy-benzylidene-cyanoacetamide a-cyano-3,4dihydroxy)-]N-benzylcinnamamide (Tyrphostin AG 490), prodigiosin 25-C (PNU156804), [4-(4′-hydroxyphenyl)-amino-6,7-dimethoxyquinazoline] (WHI-P131), [4-(3′-bromo-4′-hydroxylphenyl)-amino-6,7-dimethoxyquinazoline] (WHI-P154), [4-(3′,5′-dibromo-4′-hydroxylphenyl)-amino-6,7-dimethoxyquinazoline] WHI-P97, KRX-211, 3-{(3R,4R)4-methyl-3-[methyl-(7H-pyrrolo[2,3-d]pyrimidin4-yl)-amino]-piperidin-1-yl)-3-oxo-propionitrile, in free form or in a pharmaceutically acceptable salt form, e.g., mono-citrate (also called CP-690,550), or a compound as disclosed in WO 04/052359 or WO 05/066156); a S1P receptor agonist or modulator (e.g., FTY720 optionally phosphorylated or an analog thereof, e.g., 2-amino-2-[4-(3-benzyloxyphenylthio)-2-chlorophenyl]ethyl-1,3-propanediol optionally phosphorylated or 1-{4-[1-(4-cyclohexyl-3-trifluoromethyl-benzyloxyimino)-ethyl]-2-ethyl-benzyl}-azetidine-3-carboxylic acid or its pharmaceutically acceptable salts); immunosuppressive monoclonal antibodies (e.g., monoclonal antibodies to leukocyte receptors, e.g., MHC, CD2, CD3, CD4, CD7, CD8, CD25, CD28, CD40, CD45, CD52, CD58, CD80, CD86 or their ligands); other immunomodulatory compounds (e.g., a recombinant binding molecule having at least a portion of the extracellular domain of CTLA4 or a mutant thereof, e.g., an at least extracellular portion of CTLA4 or a mutant thereof joined to a non-CTLA4 protein sequence, e.g., CTLA41 g (for ex. designated ATCC 68629) or a mutant thereof, e.g., LEA29Y); adhesion molecule inhibitors (e.g., LFA-1 antagonists, ICAM-1 or -3 antagonists, VCAM4 antagonists or VLA-4 antagonists). These compounds or agents may also be used in combination.

Another aspect of the invention pertains to methods of modulating biomarker protein expression or activity for therapeutic purposes. Accordingly, in an exemplary embodiment, the modulatory method of the invention involves contacting a cell with a biomarker protein or agent that modulates one or more of the activities of a biomarker protein activity associated with the cell. An agent that modulates biomarker protein activity can be an agent as described herein, such as a nucleic acid or a protein, a naturally-occurring target molecule of a biomarker protein (e.g., a biomarker protein substrate), a biomarker protein antibody, a biomarker protein agonist or antagonist, a peptidomimetic of a biomarker protein agonist or antagonist, or other small molecule. In one embodiment, the agent stimulates one or more biomarker protein activities. Examples of such stimulatory agents include active biomarker protein and a nucleic acid molecule encoding biomarker protein that has been introduced into the cell. In another embodiment, the agent inhibits one or more biomarker protein activities. Examples of such inhibitory agents include antisense biomarker protein nucleic acid molecules, anti-biomarker protein antibodies, and biomarker protein inhibitors. These modulatory methods can be performed in vitro (e.g., by culturing the cell with the agent) or, alternatively, in vivo (e.g., by administering the agent to a subject). As such, the present invention provides methods of treating a subject afflicted with a disease or disorder characterized by aberrant expression or activity of a biomarker protein or nucleic acid molecule. In one embodiment, the method involves administering an agent (e.g., an agent identified by a screening assay described herein), or combination of agents that modulates (e.g., up-regulates or down-regulates) biomarker protein expression or activity. In another embodiment, the method involves administering a biomarker protein or nucleic acid molecule as therapy to compensate for reduced or aberrant biomarker protein expression or activity.

Stimulation of biomarker protein activity is desirable in situations in which biomarker protein is abnormally down-regulated and/or in which increased biomarker protein activity is likely to have a beneficial effect. For example, stimulation of biomarker protein activity is desirable in situations in which a biomarker is down-regulated and/or in which increased biomarker protein activity is likely to have a beneficial effect. Likewise, inhibition of biomarker protein activity is desirable in situations in which biomarker protein is abnormally up-regulated and/or in which decreased biomarker protein activity is likely to have a beneficial effect.

The biomarker protein and nucleic acid molecules of the present invention, as well as agents, or modulators which have a stimulatory or inhibitory effect on biomarker protein activity (e.g., biomarker gene expression), as identified by a screening assay described herein, can be administered to subjects to treat (prophylactically or therapeutically) biomarker-associated disorders (e.g., prostate cancer) associated with aberrant biomarker protein activity. In conjunction with such treatment, pharmacogenomics (i.e., the study of the relationship between a subject's genotype and that subject's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a biomarker molecule or biomarker modulator as well as tailoring the dosage and/or therapeutic regimen of treatment with a biomarker molecule or biomarker modulator.

One pharmacogenomics approach to identifying genes that predict drug response, known as “a genome-wide association”, relies primarily on a high-resolution map of the human genome consisting of already known gene-related biomarkers (e.g., a “bi-allelic” gene biomarker map which consists of 60,000-100,000 polymorphic or variable sites on the human genome, each of which has two variants). Such a high-resolution genetic map can be compared to a map of the genome of each of a statistically significant number of subjects taking part in a Phase II/III drug trial to identify biomarkers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, subjects can be grouped into genetic categories depending on a particular pattern of SNPs in their subject genome. In such a manner, treatment regimens can be tailored to groups of genetically similar subjects, taking into account traits that may be common among such genetically similar subjects.

Alternatively, a method termed the “candidate gene approach”, can be utilized to identify genes that predict drug response. According to this method, if a gene that encodes a drugs target is known (e.g., a biomarker protein of the present invention), all common variants of that gene can be fairly easily identified in the population and it can be determined if having one version of the gene versus another is associated with a particular drug response.

Information generated from more than one of the above pharmacogenomics approaches can be used to determine appropriate dosage and treatment regimens for prophylactic or therapeutic treatment of a subject. This knowledge, when applied to dosing or drug selection, can avoid adverse reactions or therapeutic failure and thus enhance therapeutic or prophylactic efficiency when treating a subject with a biomarker molecule or biomarker modulator, such as a modulator identified by one of the exemplary screening assays described herein.

This invention is further illustrated by the following examples which should not be construed as limiting. The contents of all references, patents and published patent applications cited throughout this application, are incorporated herein by reference.

### Examples

### Example 1

### Identifying Biomarkers Predictive of Chronic/Sclerosing Allograft Nephropathy

1 Introduction and Purpose of the Studies

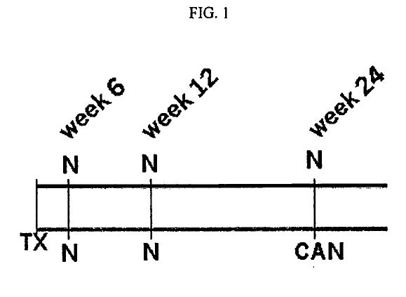

Histopathological evaluation of biopsy tissue is the gold standard of diagnosis of chronic renal allograft nephropathy (CAN), while prediction of the onset of CAN is currently impossible. Molecular diagnostics, like gene expression profiling, may aid to further refine the BANFF 97 disease classification (Racusen L C, et al., Kidney Int. 55(2):713-23 (1999)), and may also be employed as predictive or early diagnostic biomarkers when applied at early time points after transplantation when by other means graft dysfunction is not yet detectable. In the present study, gene expression profiling was applied to biopsy RNA extracted from serial renal protocol biopsies from patients which showed no overt deterioration of graft function within about at least one year after transplantation, and patients which had overt chronic allograft nephropathy (CAN) as diagnosed at the week 24 biopsy, but not at week 06 or week 12 biopsy (see FIG. 1). Specifically, to identify genomic biomarkers of chronic/sclerosing allograft nephropathy which, based on mRNA expression levels derived from kidney biopsies of renal transplant patients, allows for early detection/diagnosis (prediction) of future CAN at a time point when histopathological investigations of the same kidneys fail to diagnose CAN. Three analysis approaches were followed: (1) identification of genomic biomarker for early diagnosis (prediction) at week 06 post TX (18 weeks before histopathological diagnosis of CAN); (2) identification of genomic biomarker for early diagnosis (prediction) at week 12 post TX (12 weeks before histopathological diagnosis of CAN); and (3) identification of genomic biomarker for early diagnosis (prediction) at week 06 post TX (18 weeks before histopathological diagnosis of CAN), or week 12 post TX (12 weeks before histopathological diagnosis of CAN), or the diagnosis of CAN versus N.

1.1 Patient Stratification

Kidney biopsy samples from renal transplant patients at all three timepoints were analysed. In this study, the dataset encompassed 67 biopsy samples or subsets of these. The sample distribution across the different grades of chronic/sclerosing allograft nephropathy (CAN) is shown below in Table 3A.

TABLE 3A
Number of samples with different grade of disease
recruited from two clinical centers

| | Patient Number from |
|---|---|
| Grade of CAN | MHH |
| 0: stable graft | 33 |
| 0: Week 06: latent CAN | 8 |
| 0: Week 12: latent CAN | 8 |
| I: mild | 18 |
| Total | 67 |

The “normal” samples were stratified into the following groups as follows:

Source: patients with stable renal allograft function throughout the observation period (number of biopsy samples: 36)

Source: patients with declining renal allograft function, as diagnosed on week 24 biopsy;
Week 6 post-TX (18 weeks before histopathological evidence of CAN): 8 samples
Week 12 post-TX (12 weeks before histopathological evidence of CAN): 8 samples

The “CAN grade I” samples were obtained from patients at any time after transplantation.

TABLE 3B
Comparison of data from patients without clinical signs of rejection or
nephropathy (N = 12) and patients with overt CAN at week 24 (N = 8).

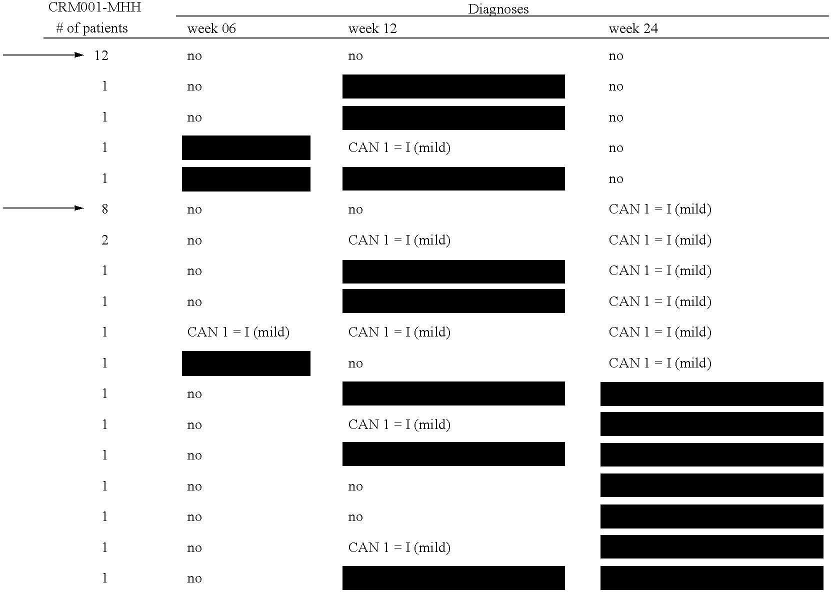

2 Sample Processing

2.1 RNA Extraction and Purification

Total RNA was obtained by acid guanidinium thiocyanate-phenol-chloroform extraction (Trizol, Invitrogen Life Technologies) from each frozen tissue section and the total RNA was then purified on an affinity resin (RNeasy, Qiagen) according to the manufacturer's instructions and quantified. Total RNA was quantified by the absorbance at λ=260 nm (A₂₆₀ₙₘ), and the purity was estimated by the ratio A_{260 nm}/A₂₈₀ₙₘ. Integrity of the RNA molecules was confirmed by non-denaturing agarose gel electrophoresis. RNA was stored at approximately −80° C. until analysis.

2.2 GeneChip Experiment

All DNA microarray experiments were conducted in the Genomics Factory EU, Basel, Switzerland, following the instructions of the manufacturer of the GeneChip system (Affymetrix, Inc., San Diego, Calif., USA) and as previously described (Lockhart D J, et al., Nat Biotechnol. 14(13):1675-80 (1996)).

Total RNA was obtained from snap frozen kidney samples by acid guanidinium isothiocyanate-phenol-chloroform extraction (Chomczynski P, et al., Anal Biochem 162(1):156-9 (1987)) using Trizol (Invitrogen Life Technologies, San Diego, Calif., USA) and was purified on an affinity resin column (RNeasy; Qiagen, Hilden, Germany) according to the manufacturer's instructions. Human HG_{—}133_plus2_target arrays [Affymetrix] were used, comprising more than 54,000 probe sets, analyzing over 35,000 transcripts and variants from over 28,000 well-substantiated human genes. One GeneChip was used per tissue, per animal. The resultant image files (.dat files) were processed using the Microarray Analysis Suite 5 (MAS5) software (Affymetrix). Tab-delimited files containing data regarding signal intensity (Signal) and categorical expression level measurement (Absolute Call) were obtained. Raw data were converted to expression levels using a “target intensity” of 150. The data were checked for quality prior to uploading to an electronic database.

2.3 Data Analysis

Data analysis was performed using Silicon Genetics software package GeneSpring version 7.2 and with SIMCA-P+ (version 11) by Umetrics AB, Sweden.

2.3.1 Filtering, Interpretation

Various filtering and clustering tools in these software packages were used to explore the datasets and identify transcript level changes that inform on altered cellular and tissue functions and that can be used to establish working hypotheses on the mode of action of the compound.

To account for experimental microarray-wide variations in intensity, all measurements on each array were normalized by dividing them by the 50th percentile of that array. Furthermore, the expression values for each gene were normalized by dividing them by the median expression value for that gene in the control group.

For the identification of the various biomarkers different filters were applied, which are described separately for each biomarker. The information content of these data, which is a conjunction of numerical changes and biological information was evaluated by comparing the data to various databases and scientific literature. Several databases were used to explore biological relevance of the datasets, e.g., PubMed (http://www.ncbi.nlm.nih.gov), NIH David (http://david.niaid.nih.gov), Affymetrix (https://www.affymetrix.com), as well internal databases. The value of that relationship was assessed by the analyst, and any hypothesis generated from this analysis would need further validation with other analytical and experimental techniques.

2.3.2 Predictive Modelling and Validation Techniques

The challenge of minimizing the trade off between goodness of fit (R2) and goodness of prediction (Q2) was addressed.

Normalized expression values were log-transformed and Pareto scaled. For some of the predictive models, the data underwent orthogonal signal correction. Partial Least Squares (PLS) was employed as supervised learning algorithms.

2.3.3 Supervised Learning by Partial Least Squares

Partial Least Squares (PLS) is one of the methods of choice when the issue is the prediction of a variable and there exist a very large number of correlated predictors. It is probably one of the best statistical approaches for prediction when there is multicollineality and a much larger number of variables than observations.

The goal of PLS regression is to provide a dimension reduction strategy in a situation where we want to relate a set of response variables Y to a set of predictor variables X. We looked for orthogonal X-components tₕ=Xwₕ* and Y-components uₕ=Ycₕ maximising the covariance between tₕ and uₕ. It was a compromise between the principal component analyses of X and Y and the canonical correlation analysis of X and Y. Note that canonical correlation analysis or multivariate regression was not directly applicable because there are many more predictors (cDNA clones) than observations; in addition, the high multicollineality observed with microarray data causes a poor performance of the multivariate regression and of canonical analysis even if a subset of expression levels were selected. The PLS methodology, in contrast, can be applied even when there are many more predictor variables than observations, as is the case with microarray data (Pérez-Encisol M, et al, Human Genetics 112(5-6):581-92 (2003)). The particular case of PLS-DA is a PLS regression where Y is a set of binary variables describing the categories of a categorical variable on X; i.e., the number dependent, or response, variables is equal to the number of categories. Alternative discrimination strategies are found in Nguyen and Rocke (Nguyen D V, et al, Bioinformatics 18:39-50 (2002)). For each response variable, yₖ, a regression model on the X-components is written:

$${y}_{k} = {\sum }_{h = 1}^{m} ( {Xw}_{h}^{*} ) {c}_{h} + e = {XW}^{*} e + e ,$$

where wₕ* is a p dimension vector containing the weights given to each original variable in the k-th component, and cₕ is the regression coefficient of yₖ on h-th X-component variable. We used the algorithm developed by Wold et al. (Wold et al., The multivariate calibration problem in chemistry solved by the PLS method. In: Ruhe A, Kagstrom B (eds) Proc Conf Matrix Pencils. Springer, Heidelberg, pp 286-293 (1983)) that allows for missing values. A fundamental requirement for PLS to yield meaningful answers is some preliminary variable selection. We did this by selecting the variables on the basis of the VIP for each variable. The VIP is a popular measure in the PLS literature and is defined for variable j as:

$${VIP}_{j} = {\{ p {\sum }_{h = 1}^{m} {\sum }_{k} {R}^{2} ( {y}_{k} , {t}_{k} ) {w}_{hj}^{2} / {\sum }_{h = 1}^{w} {\sum }_{k} {R}^{2} ( {y}_{k} , {t}_{k} ) \}}^{1 / 2} ,$$

(Eriksson L, et al., Umetrics, Umea (1999); (Tenenhaus M, La régression PLS. Editions Technip, Paris (1998)) for each j-th predictor variable J=1, p, where R²(a,b) stands for the squared correlation between items in vector a and b, and tₕ=Xₕ₋₁wₕ, where Xₕ₋₁ is the residual matrix in the regression of X on components t₁, . . . tₕ₋₁ and wₕ is a vector of norm 1 (in the PLS regression algorithm tₕ is build with this normalisation constraint). Note that wₕⱼ measures the contribution of each variable j to the h-th PLS component. Thus, VIPⱼ quantifies the influence on the response of each variable summed over all components and categorical responses (for more than two categories in Y), relative to the total sum of squares of the model; this makes the VIP an intuitively appealing measure of the global effect of each cDNA clone. The VIP has also the property of

$${\sum }_{j = 1}^{p} VIP \text{?} = p . \\ \text{?} \text{indicates text missing or illegible when filed}$$

In this work, a first analysis was carried out with all variables (cDNA levels) and the VIP was assessed for each variable. The number of PLS components was selected if a new component satisfied the Q² criterion; i.e.,

Qₕ²=1−PRESSₕ/RESSₕ₋₁≧0.05,

where PRESSₕ is the predicted sum of squares of a model containing h components, and RESSₕ₋₁ is the residual sum of squares of a model containing h−1 components. PRESS is computed by cross validation,

$${PRESS}_{h} = {\sum }_{i = 1}^{n} {( {y}_{h - 1 , i} - {\hat{y}}_{h - 1 , - i} )}^{2} ,$$

with y_{h-1,i} being the residual of observation i when h−1 components are fitted, and ýh−1−1 is the predicted yᵢ obtained when the i-th observation is removed. Prediction of a new observation is simply obtained as

$${\hat{y}}_{i} = {\sum }_{h = 1}^{m} ( \text{?} \text{?} ) {c}_{h} , \\ \text{?} \text{indicates text missing or illegible when filed}$$

where xᵢ is the vector containing the variable records for the new observation i.

Model validation was carried out via permutation. Permutation tests are part of the computer intensive procedures that have become very popular in the last years due to their flexibility and to increasing computer power (Good PI, PERMUTATION TESTS: A PRACTICAL GUIDE TO RESAMPLING METHODS FOR TESTING HYPOTHESES. Springer, New York. The principle is very simple, to test the significance of a statistic T in a given sample, the response vector (Y) N times is randomised, Tᵢ, i=1, N is computed for each of the permutation sets, and the distribution of T under the null hypothesis is approximated by the set of Tᵢ values; e.g., the 5% significance threshold will be the 0.05×N largest value of all Tᵢ. In the present example, the response vector (Y) was permuted 200 times and, redoing the analysis, the values of Q² and R² were plotted, where

$${Q}_{2} = 1 - {\prod }_{k = 1}^{m} {PRESS}_{k} \text{/} {RESS}_{k - 1}$$

and R² is the fraction of the total sums of squares explained by the model. Q² is a measurement of the predictive ability of the model, whereas R² is related to the model's goodness of fit. Analyses were done with SIMCA-P software (Eriksson L, et al., Umetrics, Umea (1999)).

3 Results

3.1 Biomarker Week 06 Post Transplantation

3.1.1 Strategy

Gene expression profiles of renal allograft biopsy samples taken at week 06 after renal transplant (“TX”) from twelve patients with stable graft function until at least 12 months post TX were compared to eight patients with declining renal graft function and histopathological diagnosis of CAN at week 24. Importantly, at time point week 06, all biopsies in this study were diagnosed as stable.

3.1.2 Data Processing

MAS5 transformed data were normalized to the 50^{th} percentile of each microarray, then normalized on the median of all normal samples from the patients with stable graft function, according to the batch of hybridization (GeneSpring Version 7.2). The gene expression intensity per patient group was calculated as the trimmed mean (Tmean) allowing one outlier sample to the top and one to the low expression range (Windows Excel 2002). Coefficient of variance (CV) was calculated as the sixth of the difference of the 20^{th} and the 80^{th} percentile of the expression range of a group, and expressed as percentage of the Tmean of that group. Only genes with coefficient of variance (CV) smaller than 20% in the group of samples from patients with longterm stable renal allografts were included in the further analysis. These genes were then filtered by the following criteria:
(1) Tmean >100 in either of the two groups
(2) p-value of ttest (two-tailed, homoscedastic) <0.05
(3) fold change between T mean of the two groups >1.2

This filter resulted in 188 probe sets.

Normalized data were subjected to predictive modelling and validation techniques (section 2.3.2, 2.3.3) to identify the best model for this dataset.

3.1.3 Biomarker Week 06 Post TX (“N2-pre-CAN” vs “N”), Result

In the present example, 49 probe sets were identified to be sufficient and necessary to predict the membership of each sample to the correct group.

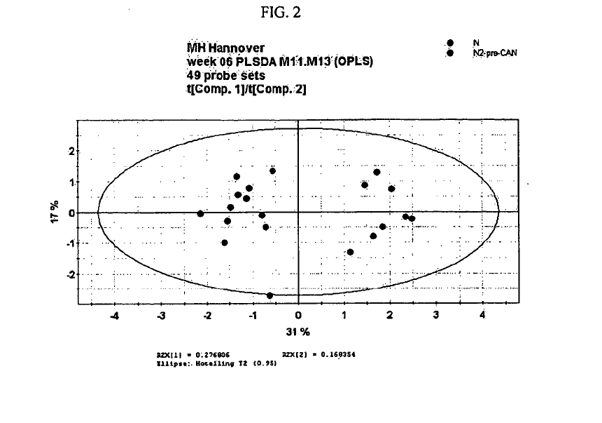

FIG. 2 is a scatter plot of the Biomarker week 06, PLS-DA model.

A scatterplot or scatter graph is a graph used in statistics to visually display and compare two sets of related quantitative, or numerical, data by displaying only finitely many points, each having a coordinate on a horizontal and a vertical axis. In FIG. 2 each dot represents a sample of a patient. Relative distance between data points is a measure of relationship/resemblance. The separation of the “N” samples from the “pre-CAN” samples indicates the potency of the algorithm/model to discriminate between the data points with the use of 49 probe sets.

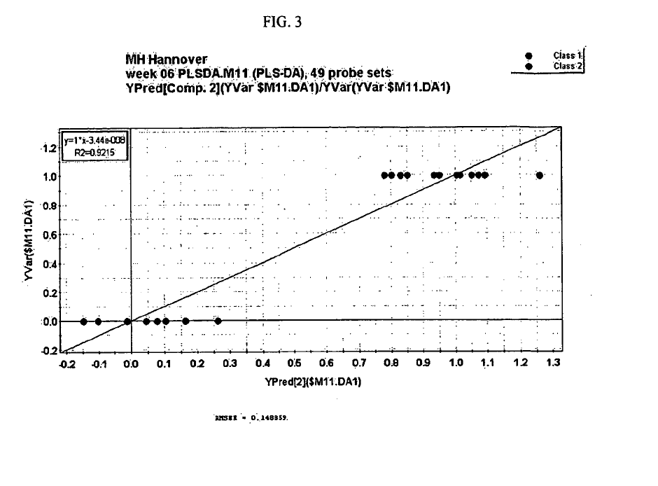

FIG. 3 is a graph comparing observed versus predicted data for the Biomarker week 06 PLSDA model.

The prediction of the Y space samples can be plotted as a scatter plot. RMSE (Root mean square error) is the standard deviation of the predicted residuals (error), and is computed as the square root of (Σ-(obs-pred)²/N). A small RMSE is a measure for a good fit of a model. The Y-axis of the plot represents the observed classes of the model, the X-axis the predicted classes. A match of Y- and X-values in this plot demonstrates the good fit of the model.

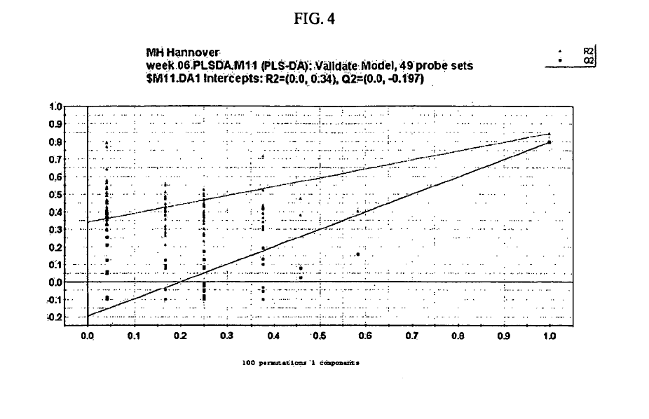

FIG. 4 shows the Biomarker week 06 PLSDA model: Validation by Response Permutation.

Validation by response permutation is an internal cross-validation, which creates a training set and a test set of samples. A model is fitted to explain the test set based on the training set and the values for R²Y (explained variance) and Q² (predicted variance) are computed and plotted. By random permutation of the training and test sets, a number of R²Y/Q² are obtained. The validate plot is then created by letting the Y-axis represent the R²Y/Q²-values of all models, including the “real” one, and by assigning the X-axis to the correlation coefficients between permuted and original response variables. A regression line is then fitted among the R²Y points and another one through the Q² points. The intercepts of the regression lines are interpretable as measures of “background” R2Y and Q² obtained to fit the data. Intercepts around 0.4 and below for R2Y and around 0.05 and below for Q² indicate valid models. Since these criteria are met in this model it is an indication of a valid model for the present dataset.

The combination of biomarker genes that form a molecular signature 6 weeks after tissue transplantation are shown in Table 4. Stable graft should describe the group values of the group of samples from patients which will not develop CAN at any later timepoint and indicates the level of expression of the genes at the “baseline” level.

TABLE 4
Genes of the Biomarker week 06, PLSDA model

| | | | | | Stable Graft: |
|---|---|---|---|---|---|
| | | | | | Raw |
| Affymetrix | | | | | Expression |
| Probe Set ID | Description | Common | Genbank | Fold change | Value |
| 221657_s_at | ankyrin repeat and SOCS | ASB6 | BC001719 | 0.72 | 127 |
| | box-containing 6 | | | | |
| 224489_at | ARF protein | LOC51326 | BC006271 | 1.52 | 74 |
| 213710_s_at | calmodulin 1 (phosphorylase | CALM1 | AL523275 | 1.53 | 142 |
| | kinase, delta) | | | | |
| 1558404_at | CDNA FLJ41173 fis, clone | | BC015390 | 0.78 | 174 |
| | BRACE2042394 | | | | |
| 201183_s_at | chromodomain helicase | CHD4 | AI613273 | 0.74 | 364 |
| | DNA binding protein 4 | | | | |
| 222809_x_at | chromosome 14 open | C14orf136 | AA728758 | 1.38 | 155 |
| | reading frame 136 | | | | |
| 222492_at | chromosome 21 open | C21orf124 | AW262867 | 0.62 | 169 |
| | reading frame 124 | | | | |
| 227188_at | chromosome 21 open | C21orf63 | AI744591 | 1.51 | 243 |
| | reading frame 63 | | | | |
| 224991_at | c-Maf-inducing protein | CMIP | AI819630 | 0.63 | 82 |
| 223495_at | coiled-coil domain | CCDC8 | AI970823 | 0.64 | 351 |
| | containing 8 | | | | |
| 239860_at | dihydropyrimidinase | DPYS | AI311917 | 0.66 | 143 |
| 212728_at | discs, large homolog 3 | DLG3 | T62872 | 0.74 | 113 |
| | (neuroendocrine-dlg, | | | | |
| | Drosophila) | | | | |
| 225167_at | FERM domain containing 4 | FRMD4 | AW515645 | 0.63 | 254 |
| 236656_s_at | Full length insert cDNA | | AW014647 | 1.34 | 276 |
| | YI37C01 | | | | |
| 213645_at | gb: AF305057 | | AF305057 | 1.39 | 387 |
| | /DB_XREF = gi: 11094017 | | | | |
| | /FEA = DNA_1 /CNT = 29 | | | | |
| | /TID = Hs.180433.1 | | | | |
| | /TIER = Stack /STK = 12 | | | | |
| | /UG = Hs.180433 /LL = 55556 | | | | |
| | /UG_GENE = HSRTSBETA | | | | |
| | /UG_TITLE = rTS beta | | | | |
| | protein /DEF = Homo sapiens | | | | |
| | RTS (RTS) gene, complete | | | | |
| | cds, alternatively spliced | | | | |
| 231951_at | guanine nucleotide binding | GNAO1 | AL512686 | 1.55 | 81 |
| | protein (G protein), alpha | | | | |
| | activating activity | | | | |
| | polypeptide O | | | | |
| 203394_s_at | hairy and enhancer of split 1, | HES1 | BE973687 | 0.72 | 618 |
| | (Drosophila) | | | | |
| 241031_at | hypothetical LOC145741 | | BE218239 | 0.78 | 80 |
| 223542_at | hypothetical protein | DKFZp761C121 | AL136560 | 0.71 | 74 |
| | DKFZp761C121 | | | | |
| 215063_x_at | hypothetical protein | FLJ20331 | AL390149 | 0.76 | 136 |
| | FLJ20331 | | | | |
| 226485_at | hypothetical protein | FLJ20674 | BG547864 | 0.71 | 278 |
| | FLJ20674 | | | | |
| 230012_at | hypothetical protein | FLJ34790 | AW574774 | 1.39 | 102 |
| | FLJ34790 | | | | |
| 1557207_s_at | hypothetical protein | LOC283177 | AI743605 | 0.72 | 152 |
| | LOC283177 | | | | |
| 225033_at | hypothetical protein | LOC286167 | AV721528 | 1.36 | 160 |
| | LOC286167 | | | | |
| 231424_at | hypothetical protein | MGC52019 | AV700405 | 2.08 | 351 |
| | MGC52019 | | | | |
| 224525_s_at | hypothetical protein PTD004 | PTD004 | AL136546 | 1.63 | 78 |
| 209291_at | inhibitor of DNA binding 4, | ID4 | AW157094 | 1.53 | 1689 |
| | dominant negative helix- | | | | |
| | loop-helix protein | | | | |
| 228002_at | isopentenyl-diphosphate | IDI2 | AI814569 | 1.44 | 104 |
| | delta isomerase 2 | | | | |
| 231850_x_at | KIAA1712 | KIAA1712 | AB051499 | 0.71 | 104 |
| 229095_s_at | LIM and senescent cell | | AI797263 | 1.83 | 135 |
| | antigen-like domains 3 | | | | |
| 229874_x_at | LOC388599 (LOC388599), | | BE865517 | 0.70 | 710 |
| | mRNA | | | | |
| 213215_at | MRNA full length insert | | AI910895 | 1.57 | 246 |
| | cDNA clone EUROIMAGE | | | | |
| | 42138 | | | | |
| 226991_at | nuclear factor of activated T- | | AA489681 | 0.68 | 92 |
| | cells, cytoplasmic, | | | | |
| | calcineurin-dependent 2 | | | | |
| 203195_s_at | nucleoporin 98 kDa | NUP98 | NM_005387 | 0.78 | 109 |
| 218414_s_at | nudE nuclear distribution | NDE1 | NM_017668 | 1.89 | 178 |
| | gene E homolog 1 (A. nidulans) | | | | |
| 206302_s_at | nudix (nucleoside | NUDT4 | NM_019094 | 0.73 | 934 |
| | diphosphate linked moiety | | | | |
| | X)-type motif 4 | | | | |
| 203118_at | proprotein convertase | PCSK7 | NM_004716 | 0.77 | 170 |
| | subtilisin/kexin type 7 | | | | |
| 203555_at | protein tyrosine phosphatase, | PTPN18 | NM_014369 | 2.39 | 83 |
| | non-receptor type 18 (brain- | | | | |
| | derived) | | | | |
| 238863_x_at | ring finger protein 135 | RNF135 | AI524240 | 0.70 | 87 |
| 215127_s_at | RNA binding motif, single | RBMS1 | AL517946 | 2.62 | 2152 |
| | stranded interacting protein 1 | | | | |
| 207939_x_at | RNA binding protein S1, | RNPS1 | NM_006711 | 0.63 | 149 |
| | serine-rich domain | | | | |
| 211325_x_at | RPL13-2 pseudogene | LOC283345 | U72518 | 0.73 | 110 |
| 225779_at | solute carrier family 27 (fatty | SLC27A4 | AK000722 | 1.32 | 85 |
| | acid transporter), member 4 | | | | |
| 235579_at | splicing factor, | SFRS2IP | AA679858 | 1.67 | 122 |
| | arginine/serine-rich 2, | | | | |
| | interacting protein | | | | |
| 1316_at | thyroid hormone receptor, | THRA | X55005mRNA | 2.47 | 115 |
| | alpha (erythroblastic | | | | |
| | leukemia viral (v-erb-a) | | | | |
| | oncogene homolog, avian) | | | | |
| 242536_at | Transcribed sequences | | AI522220 | 2.21 | 526 |
| 244018_at | Transcribed sequences | | AW451618 | 1.44 | 66 |
| 244026_at | Transcribed sequences | | BF063657 | 1.46 | 71 |
| 243514_at | WD repeat and FYVE | WDFY2 | AI475902 | 1.75 | 70 |
| | domain containing 2 | | | | |

In one embodiment, the preferred genes identified at 6 weeks include, but are not limited to, NFAT (Murphy et al., (2002) J. Immunol October 1;169(7):3717-25), Discs large 3, dlg3 (Hanada et al. (2000) Int. J. Cancer May 15;86(4):480-8), and thyroid hormone receptor alpha (Sato et al. Circ Res. (2005) September 16;97(6):550-7. Epub Aug. 11, 2005).

3.2 Biomarker Week 12 Post Transplantation

3.2.1 Strategy

Gene expression profiles of renal allograft biopsy samples taken at week 12 after renal TX from twelve patients with stable graft function until at least 12 months post TX were compared to eight patients with declining renal graft function and histopathological diagnosis of CAN at week 24. Importantly, at time point week 12, all biopsies in this study were diagnosed as stable.

3.2.2 Data Processing

MAS5 transformed data were normalized to the 50^{th} percentile of each microarray, then normalized on the median of all normal samples from the patients with stable graft function, according to the batch of hybridization (GeneSpring Version 7.2). The gene expression intensity per patient group was calculated as the trimmed mean (Tₘₑₐₙ) allowing one outlier sample to the top and one to the low expression range (Windows Excel 2002). Coefficient of variance (CV) was calculated as the sixth of the difference of the 20^{th} and the 80^{th} percentile of the expression range of a group, and expressed as percentage of the Tₘₑₐₙ of that group. Only genes with coefficient of variance (CV) smaller than 20% in the group of samples from patients with longterm stable renal allografts were included in the further analysis. These genes were then filtered by the following criteria:
(1) T mean>100 in either of the two groups
(2) p-value of ttest (two-tailed, homoscedastic) <0.05
(3) fold change between T mean of the two groups >1.5

This filter resulted in 664 probe sets. Normalized data were subjected to predictive modelling and validation techniques (section 2.3.2, 2.3.3) to identify the best model for this dataset.

3.2.3 Biomarker Week12 Post TX: OPLS Model, Result

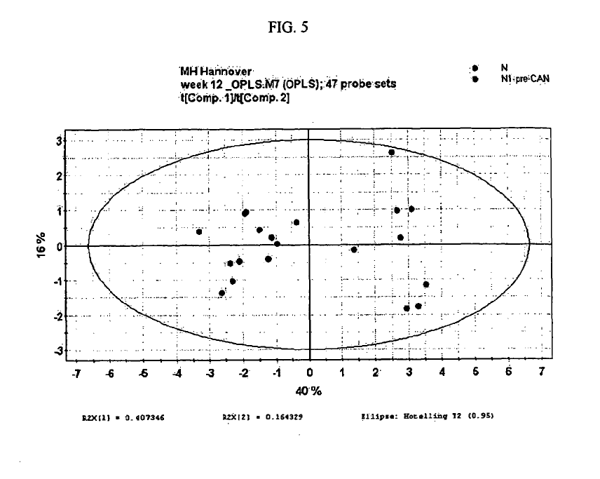

FIG. 5 shows the Biomarker week 12 OPLS model: Scatter plot.

A scatterplot or scatter graph is a graph used in statistics to visually display and compare two sets of related quantitative, or numerical, data by displaying only finitely many points, each having a coordinate on a horizontal and a vertical axis. In FIG. 5 each dot represents a sample of a patient. Relative distance between data points is a measure of relationship/resemblance. The separation of the “N” samples from the “pre-CAN” samples indicates the potency of the algorithm /model to discriminate between the data points with the use of these probe sets.

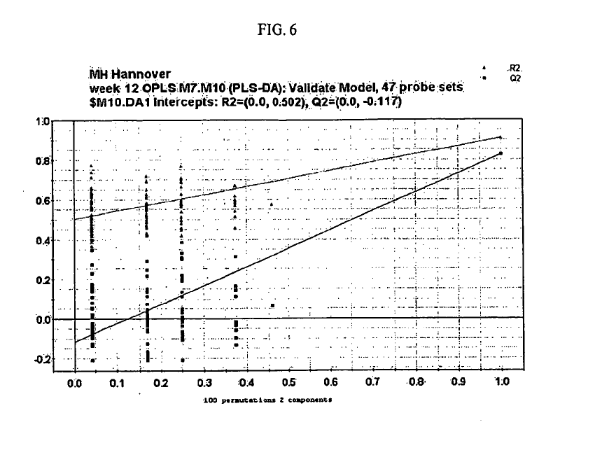

FIG. 6 shows the Biomarker week 12 OPLS model: Validation by Response Permutation.

Validation by response permutation is an internal cross-validation, which creates a training set and a test set of samples. A model is fitted to explain the test set based on the training set and the values for R²Y (explained variance) and Q² (predicted variance) are computed and plotted. By random permutation of the training and test sets, a number of R²Y/Q² are obtained. The validate plot is then created by letting the Y-axis represent the R²Y/Q²-values of all models, including the “real” one, and by assigning the X-axis to the correlation coefficients between permuted and original response variables. A regression line is then fitted among the R²Y points and another one through the Q² points. The intercepts of the regression lines are interpretable as measures of “background” R2Y and Q² obtained to fit the data. Intercepts around 0.4 and below for R2Y and around 0.05 and below for Q² indicate valid models. Since these criteria are met in this model it is an indication of a valid model for the present dataset.

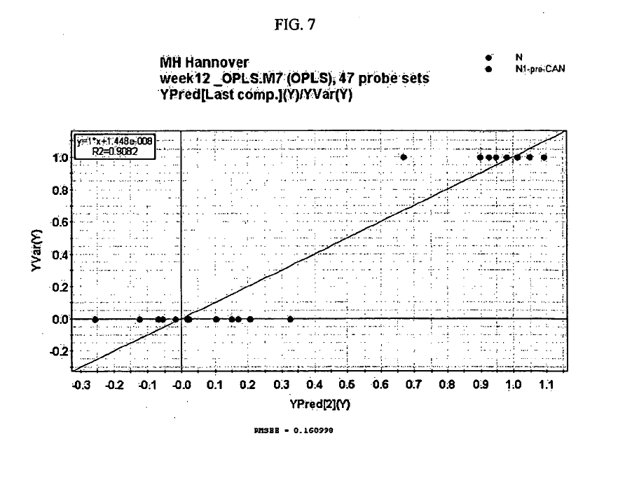

FIG. 7 shows the Biomarker week 12 OPLS model: observed vs predicted.

The prediction of the Y space samples can be plotted as a scatter plot. RMSE (Root mean square error) is the standard deviation of the predicted residuals (error), and is computed as the square root of (Σ(obs-pred)²/N). A small RMSE is a measure for a good fit of a model. The Y-axis of the plot represents the observed classes of the model, the X-axis the predicted classes. A match of Y- and X-values in this plot demonstrates the good fit of the model.

The combination of biomarker genes that form a molecular signature 12 weeks after tissue transplantation as determined by OPLS analysis are shown in Table 5.

TABLE 5
Genes of the Biomarker week 12, OPLS model

| | | | | | Stable |
|---|---|---|---|---|---|
| | | | | | Graft: Raw |
| Affymetrix | | | | Fold | Expression |
| Probe Set ID | Description | Common | Genbank | change | Value |
| 201792_at | AE binding protein 1 | AEBP1 | NM_001129 | 2.13 | 212 |
| 211712_s_at | annexin A9 | ANXA9 | BC005830 | 0.47 | 190 |
| 207367_at | ATPase, H+/K+ transporting, | ATP12A | NM_001676 | 0.48 | 108 |
| | nongastric, alpha polypeptide | | | | |
| 233085_s_at | AV734843 cdA Homo sapiens | FLJ22833 | AV734843 | 2.13 | 368 |
| | cDNA clone cdAAHD10 5′, | | | | |
| | mRNA sequence. | | | | |
| 227140_at | CDNA FLJ11041 fis, clone | | AI343467 | 1.95 | 105 |
| | PLACE1004405 | | | | |
| 232090_at | CDNA FLJ11481 fis, clone | | AI761578 | 1.89 | 102 |
| | HEMBA1001803 | | | | |
| 232991_at | CDNA FLJ11613 fis, clone | | AK021675 | 1.96 | 101 |
| | HEMBA1004012 | | | | |
| 1570198_x_at | Clone IMAGE: 5111803, | | BC019872 | 2.23 | 131 |
| | mRNA | | | | |
| 229218_at | collagen, type I, alpha 2 | COL1A2 | AA628535 | 4.04 | 212 |
| 232458_at | collagen, type III, alpha 1 | | AU146808 | 0.50 | 66 |
| | (Ehlers-Danlos syndrome type | | | | |
| | IV, autosomal dominant) | | | | |
| 201438_at | collagen, type VI, alpha 3 | COL6A3 | NM_004369 | 8.84 | 1146 |
| 226237_at | collagen, type VIII, alpha 1 | COL8A1 | AL359062 | 2.00 | 471 |
| 227336_at | deltex homolog 1 (Drosophila) | DTX1 | AW576405 | 0.42 | 125 |
| 210165_at | deoxyribonuclease I | DNASE1 | M55983 | 0.55 | 189 |
| 220625_s_at | E74-like factor 5 (ets domain | ELF5 | AF115403 | 2.26 | 405 |
| | transcription factor) | | | | |
| 221870_at | EH-domain containing 2 | EHD2 | AI417917 | 1.71 | 55 |
| 227353_at | epidermodysplasia | EVER2 | BE671663 | 2.42 | 70 |
| | verruciformis 2 | | | | |
| 242974_at | frizzled homolog 9 | FZD9 | AA446657 | 2.49 | 50 |
| | (Drosophila) | | | | |
| 211795_s_at | FYN binding protein FYB- | FYB | AF198052 | 0.40 | 89 |
| | 120/130) | | | | |
| 1560782_at | Homo sapiens cDNA clone | | BC035326 | 2.69 | 112 |
| | IMAGE: 5186324, partial cds. | | | | |
| 242372_s_at | hypothetical protein | DKFZp761N1114 | AL542291 | 2.52 | 329 |
| | DKFZp761N1114 | | | | |
| 222872_x_at | hypothetical protein FLJ22833 | FLJ22833 | AU157541 | 1.94 | 400 |
| 224489_at | hypothetical protein | LOC51326 | BC006271 | 0.45 | 94 |
| | LOC284058 | | | | |
| 212768_s_at | isoform 1 match: proteins: | GW112 | AL390736 | 2.40 | 143 |
| | Sw: Q07081 Tr: O95362 | | | | |
| | Tr: Q9Z2Y4 Tr: O95897 | | | | |
| | Tr: O70624 Sw: Q99972 | | | | |
| | Sw: Q99784 Sw: Q62609 | | | | |
| | Tr: Q9TV76 Tr: Q9I9K5 | | | | |
| | Sw: P01813 Tr: Q9IAK4 | | | | |
| | Tr: O35429; Human DNA | | | | |
| | sequence from clone RP11- | | | | |
| | 209J19 on chromosome 13 | | | | |
| | Contains ESTs, STSs and | | | | |
| | GSSs. Contains the gene for | | | | |
| | the GW112 protein with two | | | | |
| | isoforms (GW112 and | | | | |
| | KIAA4294), complete | | | | |
| | sequence. | | | | |
| 201744_s_at | lumican | LUM | NM_002345 | 2.22 | 1658 |
| 229554_at | lumican | LUM | AI141861 | 2.05 | 82 |
| 227438_at | lymphocyte alpha-kinase | LAK | AI760166 | 2.34 | 55 |
| 226841_at | macrophage expressed gene 1 | MPEG1 | BF590697 | 2.17 | 81 |
| 212999_x_at | major histocompatibility | HLA-DQB1 | AW276186 | 2.00 | 101 |
| | complex, class II, DQ beta 1 | | | | |
| 226210_s_at | maternally expressed 3 | MEG3 | AI291123 | 2.43 | 127 |
| 212012_at | Melanoma associated gene | D2S448 | BF342851 | 0.50 | 428 |
| 219666_at | membrane-spanning 4- | MS4A6A | NM_022349 | 3.20 | 157 |
| | domains, subfamily A, | | | | |
| | member 6A | | | | |
| 232113_at | MRNA; cDNA | | N90870 | 3.00 | 158 |
| | DKFZp564B182 (from clone | | | | |
| | DKFZp564B182) | | | | |
| 1556183_at | MRNA; cDNA | | AK097649 | 1.93 | 47 |
| | DKFZp686E1246 (from clone | | | | |
| | DKFZp686E1246) | | | | |
| 228055_at | napsin B pseudogene | NAP1L | AI763426 | 0.52 | 99 |
| 229070_at | ne10a12.s1 NCI_CGAP_Co3 | C6orf105 | AA470369 | 2.43 | 210 |
| | Homo sapiens cDNA clone | | | | |
| | IMAGE: 880798 3′, mRNA | | | | |
| | sequence. | | | | |
| 214111_at | opioid binding protein/cell | OPCML | AF070577 | 2.67 | 103 |
| | adhesion molecule-like | | | | |
| 205267_at | POU domain, class 2, | POU2AF1 | NM_006235 | 2.18 | 39 |
| | associating factor 1 | | | | |
| 216834_at | regulator of G-protein | RGS1 | S59049 | 1.98 | 36 |
| | signalling 1 | | | | |
| 218870_at | Rho GTPase activating protein | ARHGAP15 | NM_018460 | 2.79 | 56 |
| | 15 | | | | |
| 237639_at | SRSR846 | | AI913600 | 1.92 | 372 |
| 209374_s_at | synonym: MU; Homo sapiens | IGHM | BC001872 | 2.07 | 84 |
| | immunoglobulin heavy | | | | |
| | constant mu, mRNA (cDNA | | | | |
| | clone MGC: 1228 | | | | |
| | IMAGE: 3544448), complete | | | | |
| | cds. | | | | |
| 236203_at | te62a03.x1 | | AI377755 | 2.84 | 51 |
| | Soares_NFL_T_GBC_S1 | | | | |
| | Homo sapiens cDNA clone | | | | |
| | IMAGE: 2091244 3′ similar to | | | | |
| | gb: J02931 TISSUE FACTOR | | | | |
| | PRECURSOR (HUMAN);, | | | | |
| | mRNA sequence. | | | | |
| 203083_at | thrombospondin 2 | THBS2 | NM_003247 | 0.42 | 403 |
| 244061_at | Transcribed sequences | | AI510829 | 0.45 | 32 |
| 209960_at | unnamed protein product; | HGF | X16323 | 2.46 | 119 |
| | HGF (AA 1-728); Human | | | | |
| | mRNA for hepatocyte growth | | | | |
| | factor (HGF). | | | | |
| 202664_at | Wiskott-Aldrich syndrome | WASPIP | AW058622 | 2.71 | 385 |
| | protein interacting protein | | | | |

3.2.4 Biomarker Week12 Post TX (“N1-pre-CAN vs N”): PLSDA Model, Result

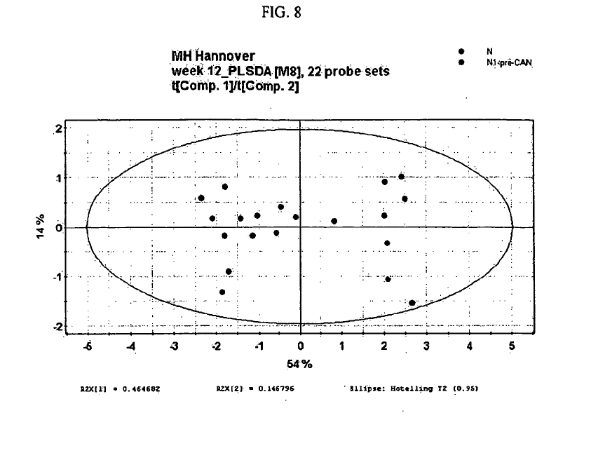

FIG. 8 shows a Biomarker week 12 PLSDA model: Scatter plot. A scatterplot or scatter graph is a graph used in statistics to visually display and compare two sets of related quantitative, or numerical, data by displaying only finitely many points, each having a coordinate on a horizontal and a vertical axis. In FIG. 8 each dot represents a sample of a patient. Relative distance between data points is a measure of relationship/resemblance. The separation of the “N” samples from the “pre-CAN” samples indicates the potency of the algorithm /model to discriminate between the data points with the use of these probe sets.

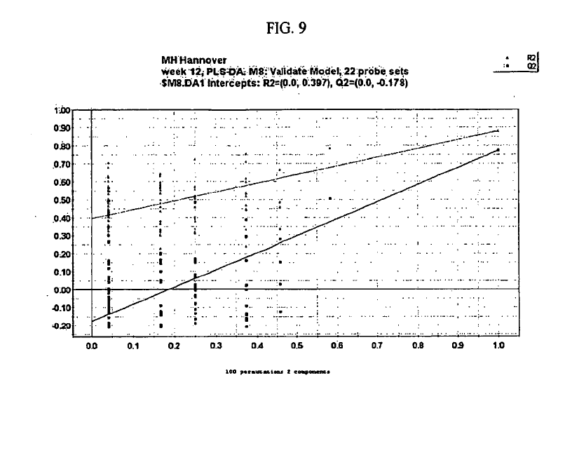

FIG. 9 shows the Biomarker week 12 PLSDA model: Validation by Response Permutation.

Validation by response permutation is an internal cross-validation, which creates a training set and a test set of samples. A model is fitted to explain the test set based on the training set and the values for R²Y (explained variance) and Q² (predicted variance) are computed and plotted. By random permutation of the training and test sets, a number of R²Y/Q² are obtained. The validate plot is then created by letting the Y-axis represent the R²Y/Q²-values of all models, including the “real” one, and by assigning the X-axis to the correlation coefficients between permuted and original response variables. A regression line is then fitted among the R²Y points and another one through the Q² points. The intercepts of the regression lines are interpretable as measures of “background” R2Y and Q² obtained to fit the data. Intercepts around 0.4 and below for R2Y and around 0.05 and below for Q² indicate valid models. Since these criteria are met in this model it is an indication of a valid model for the present dataset.

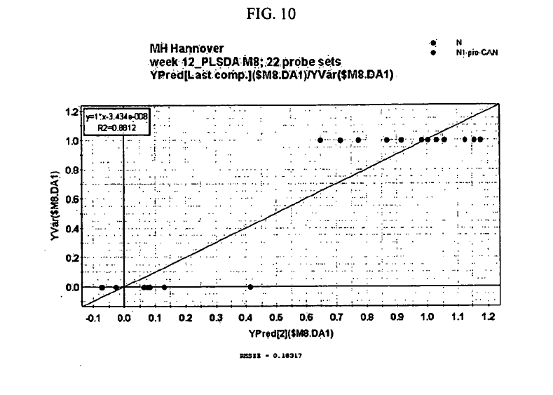

FIG. 10 shows the Biomarker week 12 PLSDA model: observed vs predicted.

The prediction of the Y space samples can be plotted as a scatter plot. RMSE (Root mean square error) is the standard deviation of the predicted residuals (error), and is computed as the square root of (Σ(obs-pred)²/N). A small RMSE is a measure for a good fit of a model. The Y-axis of the plot represents the observed classes of the model, the X-axis the predicted classes. A match of Y- and X-values in this plot demonstrates the good fit of the model.

The combination of biomarker genes that form a molecular signature 12 weeks after tissue transplantation as determined by PLSDA analysis are shown in Table 6.

TABLE 6
Genes of the Biomarker week 12, PLSDA model

| | | | | | Stable Graft: |
|---|---|---|---|---|---|
| | | | | | Raw |
| Affymetrix | | | | Fold | Expression |
| Probe Set ID | Description | Common | Genbank | change | Value |
| 201792_at | AE binding protein 1 | AEBP1 | NM_001129 | 8.84 | 212 |
| 242974_at | CD47 antigen (Rh-related | CD47 | AA446657 | 4.04 | 50 |
| | antigen, integrin-associated | | | | |
| | signal transducer) | | | | |
| 227140_at | CDNA FLJ11041 fis, clone | | AI343467 | 3.20 | 105 |
| | PLACE1004405 | | | | |
| 232090_at | CDNA FLJ11481 fis, clone | | AI761578 | 3.00 | 102 |
| | HEMBA1001803 | | | | |
| 229218_at | collagen, type I, alpha 2 | COL1A2 | AA628535 | 2.67 | 212 |
| 232458_at | collagen, type III, alpha 1 | COL3A1 | AU146808 | 0.47 | 66 |
| | (Ehlers-Danlos syndrome | | | | |
| | type IV, autosomal | | | | |
| | dominant) | | | | |
| 227336_at | deltex homolog 1 | DTX1 | AW576405 | 2.84 | 125 |
| | (Drosophila) | | | | |
| 210165_at | deoxyribonuclease I | DNASE1 | M55983 | 2.42 | 189 |
| 227353_at | epidermodysplasia | EVER2 | BE671663 | 2.46 | 70 |
| | verruciformis 2 | | | | |
| 1560782_at | Homo sapiens cDNA clone | C22orf1; 239AB; | BC035326 | 0.42 | 112 |
| | IMAGE: 5186324, partial | FAM1A | | | |
| | cds. | | | | |
| 242372_s_at | hypothetical protein | DKFZp761N1114 | AL542291 | 2.79 | 329 |
| | DKFZp761N1114 | | | | |
| 222872_x_at | hypothetical protein | FLJ22833 | AU157541 | 2.18 | 400 |
| | FLJ22833 | | | | |
| 212768_s_at | isoform 1 match: proteins: | bA209J19.1 | AL390736 | 2.43 | 143 |
| | Sw: Q07081 Tr: O95362 | | | | |
| | Tr: Q9Z2Y4 Tr: O95897 | | | | |
| | Tr: O70624 Sw: Q99972 | | | | |
| | Sw: Q99784 Sw: Q62609 | | | | |
| | Tr: Q9TV76 Tr: Q9I9K5 | | | | |
| | Sw: P01813 Tr: Q9IAK4 | | | | |
| | Tr: O35429; Human DNA | | | | |
| | sequence from clone RP11- | | | | |
| | 209J19 on chromosome 13 | | | | |
| | Contains ESTs, STSs and | | | | |
| | GSSs. Contains the gene for | | | | |
| | the GW112 protein with two | | | | |
| | isoforms (GW112 and | | | | |
| | KIAA4294), complete | | | | |
| | sequence. | | | | |
| 229554_at | lumican | LUM | AI141861 | 2.43 | 82 |
| 227438_at | lymphocyte alpha-kinase | LAK | AI760166 | 2.52 | 55 |
| 226210_s_at | maternally expressed 3 | MEG3 | AI291123 | 2.34 | 127 |
| 205267_at | POU domain, class 2, | POU2AF1 | NM_006235 | 2.23 | 39 |
| | associating factor 1 | | | | |
| 218870_at | Rho GTPase activating | ARHGAP15 | NM_018460 | 0.45 | 56 |
| | protein 15 | | | | |
| 237639_at | SRSR846 | UNQ846 | AI913600 | 0.42 | 372 |
| 209374_s_at | synonym: MU; Homo | IGHM; MU | BC001872 | 2.22 | 84 |
| | sapiens immunoglobulin | | | | |
| | heavy constant mu, mRNA | | | | |
| | (cDNA clone MGC: 1228 | | | | |
| | IMAGE: 3544448), complete | | | | |
| | cds. | | | | |
| 236203_at | te62a03.x1 | | AI377755 | 0.50 | 51 |
| | Soares_NFL_T_GBC_S1 | | | | |
| | Homo sapiens cDNA clone | | | | |
| | IMAGE: 2091244 3′ similar | | | | |
| | to gb: J02931 TISSUE | | | | |
| | FACTOR PRECURSOR | | | | |
| | (HUMAN);, mRNA | | | | |
| | sequence. | | | | |
| 203083_at | thrombospondin 2 | THBS2 | NM_003247 | 1.89 | 403 |

In one embodiment, the preferred genes identified at 12 weeks include, but are not limited to, lumican (Onda et al. Exp. Mol. Pathol. (2002) April;72(2):142-9), Smad3 (Saika et al., Am. J. Pathol. (2004) February;164(2):651-63), AE binding protein 1 (Layne et al. J. Biol. Chem. (1998) June 19;273(25):15654-60), and frizzled-9 (Karasawa et al. (2002) J. Biol. Chem October 4;277(40):37479-86. Epub Jul. 22, 2002.).

3.3 Biomarker “Global Analysis”: Identification of Genomic Predictive Biomarker Before and at Week 24 After Renal Transplantation

3.3.1 Strategy

Gene expression profiles of serial renal protocol biopsy samples taken at week 12 after renal TX from eight patients with declining renal graft function and histopathological diagnosis of CAN at week 24 were compared to 33 renal biopsy samples from patients with stable allograft function at least until 12 months post TX, and 18 biopsies with histological evidence of CAN grade 1. Classes of samples were defined as:
N (normal; longterm stable renal allograft): n=33

Week 06 (biopsy from a healthy patient who develops overt CAN between week 12 and week 24 post TX): n=8

Week 12 (biopsy from a healthy patient who develops overt CAN between week 12 and week 24 post TX): n=8

CAN: histopathological evidence of chronic allograft nephropathy: n=18.

3.3.2 Data Processing

MAS5 transformed data were normalized to the 50^{th} percentile of each microarray, then normalized by time point and batch on the median of all normal samples (n=33) from the patients with stable graft function, according to the batch of hybridization (GeneSpring Version 7.2). Only probe sets with raw expression intensity of at least 100 in at least 25% of the samples (n=18) were included in the following analysis (20,549 probe sets).

These probe sets were subjected to a Fisher's Exact Test to find an association between gene expression changes and class membership. The Find Significant Parameters using an Association Test option performs an association test for each gene, over all parameters and attributes. Both numeric and non-numeric parameters and attributes can be tested.

In this analysis the groups were defined as described in section 1.1. The test resulted in a list of 578 probe sets with a correlation of <0.0001 with the class membership described in section 1.1. Normalized data were subjected to predictive modelling and validation techniques (section 2.3.2, 2.3.3) to identify the best model for this dataset.

3.3.3 Biomarker “Global Analysis”; OSC Model, Result

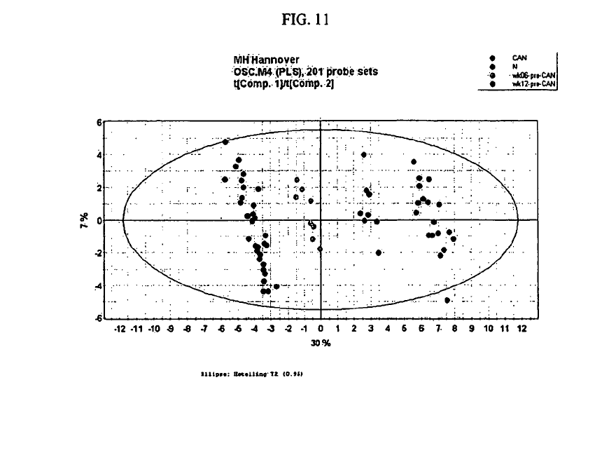

FIG. 11 shows the Biomarker global analysis OSC model: Scatter plot. A scatterplot or scatter graph is a graph used in statistics to visually display and compare two sets of related quantitative, or numerical, data by displaying only finitely many points, each having a coordinate on a horizontal and a vertical axis. In FIG. 11 each dot represents a sample of a patient. Relative distance between data points is a measure of relationship/resemblance. The separation of the “N” samples from the “week 06 pre-CAN”, “week 12 pre-CAN” and “CAN” samples indicates the potency of the algorithm /model to discriminate between the data points with the use of these probe sets.

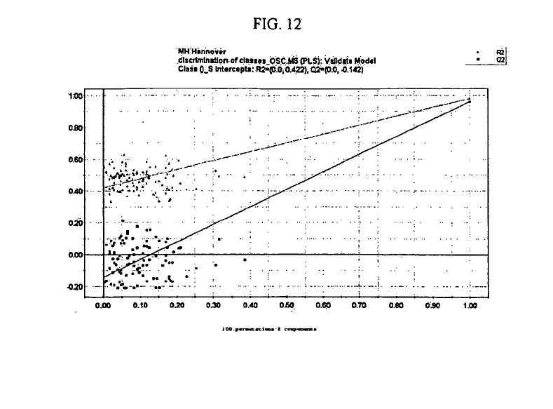

FIG. 12 shows the Biomarker global analysis OSC model: Validation by response permutation. Validation by response permutation is an internal cross-validation, which creates a training set and a test set of samples. A model is fitted to explain the test set based on the training set and the values for R²Y (explained variance) and Q² (predicted variance) are computed and plotted. By random permutation of the training and test sets, a number of R²Y/Q² are obtained. The validate plot is then created by letting the Y-axis represent the R²Y/Q²-values of all models, including the “real” one, and by assigning the X-axis to the correlation coefficients between permuted and original response variables. A regression line is then fitted among the R²Y points and another one through the Q² points. The intercepts of the regression lines are interpretable as measures of “background” R2Y and Q² obtained to fit the data. Intercepts around 0.4 and below for R2Y and around 0.05 and below for Q² indicate valid models. Since these criteria are met in this model it is an indication of a valid model for the present dataset.

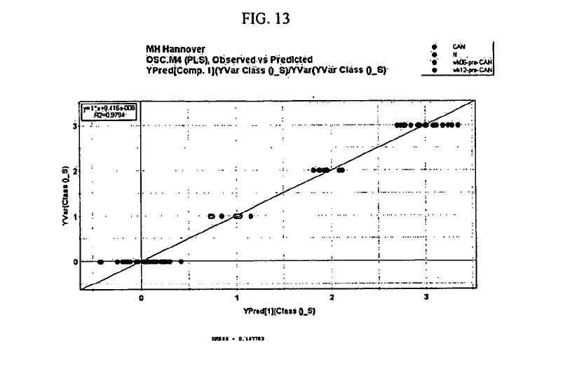

FIG. 13 Biomarker global analysis OSC model: Observed vs. predicted. The prediction of the Y space samples can be plotted as a scatter plot. RMSE (Root mean square error) is the standard deviation of the predicted residuals (error), and is computed as the square root of (Σ(obs-pred)²/N). A small RMSE is a measure for a good fit of a model. The Y-axis of the plot represents the observed classes of the model, the X-axis the predicted classes. A match of Y- and X-values in this plot demonstrates the good fit of the model.

The combination of biomarker genes that form a molecular signature after tissue transplantation as determined by global data analysis using OSC model are shown in Table 7.

TABLE 7
Genes of Biomarker Global Analysis, OSC Model

| | | | | Fold | Fold | | Stable |
|---|---|---|---|---|---|---|---|
| | | | | change | change | Fold | Graft: Raw |
| Affymetrix | | | | wk06- | wk12- | change | Expression |
| Probe Set ID | Description | Common | Genbank | pre-CAN | pre-CAN | CAN | Value |
| 244567_at | 602343781F1 NIH_MGC_89 | | BG165613 | 1.51 | 1.21 | 1.71 | 103 |
| | Homo sapiens cDNA clone | | | | | | |
| | IMAGE: 4453556 5′, mRNA | | | | | | |
| | sequence. | | | | | | |
| 244145_at | 602371458F1 NIH_MGC_93 | | BG260337 | 1.49 | 1.58 | 1.52 | 102 |
| | Homo sapiens cDNA clone | | | | | | |
| | IMAGE: 4479327 5′, mRNA | | | | | | |
| | sequence. | | | | | | |
| 201660_at | acyl-CoA Synthetase long- | ACSL3 | AL525798 | 1.94 | 2.28 | 1.91 | 876 |
| | chain family member 3 | | | | | | |
| 232175_at | ADP-ribosylation factor 1 | ARF1 | AI972094 | 1.43 | 1.58 | 1.78 | 108 |
| 232865_at | ALL1 fused gene from 5q31 | AF5Q31 | N59653 | 1.55 | 1.51 | 1.97 | 179 |
| 236778_at | alpha thalassemia/mental | ATRX | AA826176 | 1.08 | 1.17 | 1.87 | 77 |
| | retardation syndrome X- | | | | | | |
| | linked (RAD54 homolog, S. cerevisiae) | | | | | | |
| 1563792_at | amnionless homolog (mouse) | AMN | AK092824 | 1.37 | 1.57 | 1.81 | 98 |
| 226718_at | amphoterin-induced gene | KIAA1163 | AA001423 | 1.12 | 1.24 | 1.37 | 142 |
| 227260_at | ankyrin repeat domain 10 | ANKRD10 | AV724266 | 1.32 | 1.59 | 1.54 | 708 |
| 230972_at | ankyrin repeat domain 9 | ANKRD9 | AW194999 | 1.16 | 1.33 | 1.66 | 656 |
| 206993_at | ATP synthase, H+ | ATP5S | NM_015684 | 1.27 | 1.53 | 1.52 | 119 |
| | transporting, mitochondrial F0 | | | | | | |
| | complex, subunit s (factor B) | | | | | | |
| 204719_at | ATP-binding cassette, sub- | ABCA8 | NM_007168 | 0.81 | 0.65 | 0.65 | 350 |
| | family A (ABC1), member 8 | | | | | | |
| 233271_at | AU145563 HEMBA1 Homo | | AU145563 | 1.18 | 1.95 | 1.50 | 143 |
| | sapiens cDNA clone | | | | | | |
| | HEMBA1005133 3′, mRNA | | | | | | |
| | sequence. | | | | | | |
| 215204_at | AU147295 MAMMA1 Homo | | AU147295 | 1.99 | 2.06 | 3.37 | 90 |
| | sapiens cDNA clone | | | | | | |
| | MAMMA1000264 3′, mRNA | | | | | | |
| | sequence. | | | | | | |
| 236892_s_at | B1 for mucin | HAB1 | BF590528 | 1.34 | 1.25 | 1.45 | 312 |
| 227896_at | BRCA2 and CDKN1A | BCCIP | AI373643 | 1.31 | 1.27 | 2.56 | 223 |
| | interacting protein | | | | | | |
| 223679_at | catenin (cadherin-associated | CTNNB1 | AF130085 | 1.64 | 1.73 | 1.58 | 146 |
| | protein), beta 1, 88 kDa | | | | | | |
| 233019_at | CCR4-NOT transcription | CNOT7 | AU145061 | 1.17 | 1.32 | 1.59 | 89 |
| | complex, subunit 7 | | | | | | |
| 233399_x_at | CDNA clone | | AU145662 | 1.60 | 1.66 | 1.95 | 183 |
| | IMAGE: 30352956, partial cds | | | | | | |
| 232351_at | CDNA FLJ10150 fis, clone | | AK022308 | 1.54 | 1.76 | 1.70 | 152 |
| | HEMBA1003395 | | | | | | |
| 234074_at | CDNA FLJ10946 fis, clone | | AU155494 | 1.29 | 1.15 | 1.76 | 99 |
| | PLACE1000005 | | | | | | |
| 232544_at | CDNA FLJ11572 fis, clone | | AU144916 | 0.89 | 0.77 | 0.69 | 231 |
| | HEMBA1003373 | | | | | | |
| 232991_at | CDNA FLJ11613 fis, clone | | AK021675 | 0.91 | 0.81 | 0.79 | 107 |
| | HEMBA1004012 | | | | | | |
| 232952_at | CDNA FLJ11942 fis, clone | | AU146493 | 0.83 | 0.75 | 0.74 | 83 |
| | HEMBB1000652 | | | | | | |
| 230791_at | CDNA FLJ12033 fis, clone | | AU146924 | 1.37 | 1.58 | 1.43 | 241 |
| | HEMBB1001899 | | | | | | |
| 233296_x_at | CDNA FLJ12131 fis, clone | | AU147291 | 0.89 | 0.81 | 0.71 | 425 |
| | MAMMA1000254 | | | | | | |
| 233498_at | CDNA FLJ14142 fis, clone | | AK024204 | 0.58 | 0.61 | 0.68 | 282 |
| | MAMMA1002880 | | | | | | |
| 230986_at | CDNA FLJ30065 fis, clone | | AI821447 | 0.95 | 0.83 | 0.73 | 96 |
| | ADRGL2000328 | | | | | | |
| 241941_at | CDNA FLJ31511 fis, clone | | AA778747 | 0.94 | 0.84 | 0.67 | 75 |
| | NT2RI1000035 | | | | | | |
| 1557270_at | CDNA FLJ36375 fis, clone | | AA632049 | 1.21 | 1.55 | 1.72 | 283 |
| | THYMU2008226 | | | | | | |
| 235028_at | CDNA FLJ46440 fis, clone | | BG288330 | 0.81 | 0.72 | 0.49 | 659 |
| | THYMU3016022 | | | | | | |
| 234604_at | CDNA: FLJ21228 fis, clone | | AK024881 | 0.68 | 0.69 | 0.64 | 62 |
| | COL00739 | | | | | | |
| 233824_at | CDNA: FLJ21428 fis, clone | | AK025081 | 0.91 | 0.80 | 0.76 | 114 |
| | COL04203 | | | | | | |
| 228143_at | ceruloplasmin (ferroxidase) | CP | AI684991 | 1.44 | 5.78 | 3.93 | 69 |
| 223191_at | chromosome 14 open reading | C14orf112 | AF151037 | 0.68 | 0.73 | 0.58 | 541 |
| | frame 112 | | | | | | |
| 218453_s_at | chromosome 6 open reading | C6orf35 | NM_018452 | 1.56 | 2.02 | 1.59 | 110 |
| | frame 35 | | | | | | |
| 229012_at | chromosome 9 open reading | C9orf24 | AW269443 | 0.77 | 0.58 | 0.41 | 142 |
| | frame 24 | | | | | | |
| 1552455_at | chromosome 9 open reading | C9orf65 | NM_138818 | 1.23 | 1.31 | 1.48 | 81 |
| | frame 65 | | | | | | |
| 225377_at | chromosome 9 open reading | C9orf86 | BE783949 | 0.81 | 0.80 | 0.76 | 173 |
| | frame 86 | | | | | | |
| 239683_at | citrate lyase beta like | CLYBL | AI476268 | 0.98 | 1.01 | 0.67 | 243 |
| 215504_x_at | Clone 25061 mRNA sequence | | AF131777 | 1.04 | 1.17 | 1.45 | 482 |
| 243329_at | Clone IMAGE: 121662 | | AI074450 | 1.33 | 1.65 | 1.62 | 195 |
| | mRNA sequence | | | | | | |
| 231808_at | Clone IMAGE: 5302006, | | AY007106 | 1.04 | 1.54 | 1.44 | 213 |
| | mRNA | | | | | | |
| 225288_at | collagen, type XXVII, alpha 1 | COL27A1 | AI949136 | 1.13 | 1.37 | 1.47 | 304 |
| 211025_x_at | cytochrome c oxidase subunit | COX5B | BC006229 | 1.28 | 1.14 | 1.49 | 1299 |
| | Vb | | | | | | |
| 1556820_a_at | deleted in lymphocytic | DLEU2 | H48516 | 1.36 | 1.37 | 1.78 | 67 |
| | leukemia, 2 | | | | | | |
| 1556821_x_at | deletcd in lymphocytic | DLEU2 | H48516 | 1.31 | 1.33 | 1.55 | 100 |
| | leukemia, 2 | | | | | | |
| 210165_at | deoxyribonuclease 1 | DNASE1 | M55983 | 1.22 | 1.16 | 1.55 | 149 |
| 218650_at | DiGeorge syndrome critical | DGCR8 | NM_022775 | 1.41 | 1.56 | 1.64 | 167 |
| | region gene 8 | | | | | | |
| 223763_at | dystrobrevin binding protein 1 | DTNBP1 | AL136637 | 1.10 | 1.16 | 1.44 | 82 |
| 227353_at | epidermodysplasia | EVER2 | BE671663 | 1.41 | 1.59 | 2.19 | 85 |
| | verruciformis 2 | | | | | | |
| 236520_at | EST384471 MAGE | | AW972380 | 1.25 | 1.24 | 1.66 | 128 |
| | resequences, MAGL Homo | | | | | | |
| | sapiens cDNA, mRNA | | | | | | |
| | sequence. | | | | | | |
| 214805_at | eukaryotic translation | EIF4A1 | U79273 | 1.24 | 1.25 | 1.61 | 153 |
| | initiation factor 4A, isoform 1 | | | | | | |
| 242029_at | FAD104 | FAD104 | N32832 | 0.87 | 0.75 | 0.76 | 96 |
| 243649_at | F-box only protein 7 | FBXO7 | AI678692 | 0.91 | 0.75 | 0.74 | 71 |
| 230389_at | formin binding protein 1 | FNBP1 | BE046511 | 0.90 | 0.85 | 0.72 | 188 |
| 227163_at | glutathione S-transferase | GSTO2 | AL162742 | 0.71 | 0.72 | 0.67 | 361 |
| | omega 2 | | | | | | |
| 215203_at | golgi autoantigen, golgin | GOLGA4 | AW438464 | 1.25 | 1.44 | 1.36 | 109 |
| | subfamily a, 4 | | | | | | |
| 229255_x_at | golgi SNAP receptor complex | GOSR2 | BF593917 | 0.81 | 0.77 | 0.75 | 142 |
| | member 2 | | | | | | |
| 227085_at | H2A histone family, member V | H2AV | AI823792 | 0.77 | 0.69 | 0.64 | 234 |
| 240405_at | H326 | H326 | AA707411 | 0.87 | 1.16 | 1.40 | 61 |
| 203394_s_at | hairy and enhancer of split 1, | HES1 | BE973687 | 0.78 | 0.80 | 0.70 | 703 |
| | (Drosophila) | | | | | | |
| 209960_at | hepatocyte growth factor | HGF | X16323 | 1.31 | 1.54 | 1.55 | 118 |
| | (hepapoietin A; scatter factor) | | | | | | |
| 213359_at | heterogeneous nuclear | HNRPD | W74620 | 1.47 | 1.66 | 1.96 | 207 |
| | ribonucleoprotein D (AU-rich | | | | | | |
| | element RNA binding protein | | | | | | |
| | 1, 37 kDa) | | | | | | |
| 215553_x_at | Homo sapiens cDNA | | AK024315 | 1.03 | 1.34 | 1.69 | 262 |
| | FLJ14253 fis, clone | | | | | | |
| | OVARC1001376. | | | | | | |
| 233813_at | Homo sapiens cDNA: | | AK026900 | 1.13 | 1.20 | 1.57 | 76 |
| | FLJ23247 fis, clone | | | | | | |
| | COL03425. | | | | | | |
| 227298_at | Hypothetical gene supported | | AI806330 | 1.63 | 2.06 | 1.45 | 167 |
| | by AK095117 (LOC401264), | | | | | | |
| | mRNA | | | | | | |
| 237108_x_at | hypothetical protein | DKFZp761G0122 | AW611845 | 0.83 | 0.82 | 0.70 | 276 |
| | DKFZp761G0122 | | | | | | |
| 219074_at | hypothetical protein | FLJ10846 | NM_018241 | 1.41 | 1.52 | 1.64 | 418 |
| | FLJ10846 | | | | | | |
| 1557828_a_at | hypothetical protein | FLJ21657 | BE675061 | 0.81 | 0.69 | 0.72 | 148 |
| | FLJ21657 | | | | | | |
| 222872_x_at | hypothetical protein | FLJ22833 | AU157541 | 1.17 | 1.48 | 1.40 | 456 |
| | FLJ22833 | | | | | | |
| 233085_s_at | hypothetical protein | FLJ22833 | AV734843 | 1.21 | 1.37 | 1.44 | 415 |
| | FLJ22833 | | | | | | |
| 229145_at | hypothetical protein | LOC119504 | AA541762 | 1.19 | 1.25 | 1.39 | 659 |
| | LOC119504 | | | | | | |
| 227550_at | hypothetical protein | LOC143381 | AW242720 | 1.01 | 1.07 | 1.36 | 222 |
| | LOC143381 | | | | | | |
| 227415_at | hypothetical protein | LOC283508 | BF109303 | 1.59 | 1.37 | 1.99 | 350 |
| | LOC283508 | | | | | | |
| 232288_at | hypothetical protein | LOC283970 | AK026209 | 4.60 | 6.51 | 13.54 | 77 |
| | LOC283970 | | | | | | |
| 226901_at | hypothetical protein | LOC284018 | AI214996 | 0.81 | 0.86 | 0.65 | 342 |
| | LOC284018 | | | | | | |
| 235482_at | hypothetical protein | LOC285002 | BE886868 | 0.82 | 0.82 | 0.73 | 132 |
| | LOC285002 | | | | | | |
| 227466_at | hypothetical protein | LOC285550 | BF108695 | 0.86 | 0.77 | 0.74 | 589 |
| | LOC285550 | | | | | | |
| 228040_at | hypothetical protein | LOC286286 | AW294192 | 1.19 | 1.40 | 1.49 | 468 |
| | LOC286286 | | | | | | |
| 1569189_at | hypothetical protein | MGC29649 | AF289605 | 0.77 | 0.76 | 0.67 | 75 |
| | MGC29649 | | | | | | |
| 225065_x_at | hypothetical protein | MGC40157 | AI826279 | 0.80 | 0.76 | 0.75 | 237 |
| | MGC40157 | | | | | | |
| 229444_at | hypothetical protein | MGC4614 | AI051046 | 0.82 | 0.73 | 0.77 | 198 |
| | MGC4614 | | | | | | |
| 218750_at | hypothetical protein | MGC5306 | NM_024116 | 1.26 | 1.99 | 1.55 | 239 |
| | MGC5306 | | | | | | |
| 223797_at | hypothetical protein PRO2852 | PRO2852 | AF130079 | 0.81 | 0.74 | 0.14 | 169 |
| 235756_at | IL2-UM0076-240300-056- | | AW802645 | 1.81 | 1.97 | 1.66 | 75 |
| | G02 UM0076 Homo sapiens | | | | | | |
| | cDNA, mRNA sequence. | | | | | | |
| 239842_x_at | IMAGE: 20075 Soares infant | | W18186 | 0.89 | 0.80 | 0.75 | 190 |
| | brain 1NIB Homo sapiens | | | | | | |
| | cDNA clone IMAGE: 20075, | | | | | | |
| | mRNA sequence. | | | | | | |
| 209374_s_at | immunoglobulin heavy | IGHM | BC001872 | 0.83 | 0.79 | 0.73 | 123 |
| | constant mu | | | | | | |
| 242903_at | interferon gamma receptor 1 | | AI458949 | 1.56 | 1.82 | 2.00 | 90 |
| 229310_at | kelch repeat and BTB (POZ) | KBTBD9 | BE465475 | 0.86 | 0.84 | 0.76 | 175 |
| | domain containing 9 | | | | | | |
| 236368_at | KIAA0368 | | BF059292 | 1.40 | 3.18 | 1.82 | 142 |
| 216000_at | KIAA0484 protein | KIAA0484 | AA732995 | 1.20 | 1.26 | 1.45 | 74 |
| 231956_at | KIAA1618 | KIAA1618 | AA976354 | 1.62 | 2.80 | 1.80 | 111 |
| 238087_at | kinesin family member 2C | KIF2C | AI587389 | 0.82 | 0.83 | 0.74 | 92 |
| 1555929_s_at | laa10f11.x1 8 5 week embryo | | BM873997 | 1.23 | 1.78 | 1.84 | 230 |
| | anterior tongue 8 5 EAT | | | | | | |
| | Homo sapiens cDNA 3′, | | | | | | |
| | mRNA sequence. | | | | | | |
| 1557360_at | leucine-rich PPR-motif | LRPPRC | CA430402 | 1.33 | 1.26 | 1.48 | 103 |
| | containing | | | | | | |
| 1569003_at | likely ortholog of rat vacuole | VMP1 | AL541655 | 0.85 | 0.82 | 0.73 | 213 |
| | membrane protein 1 | | | | | | |
| 223223_at | likely ortholog of yeast ARV1 | ARV1 | AF321442 | 1.23 | 1.37 | 1.58 | 520 |
| 227438_at | lymphocyte alpha-kinase | LAK | AI760166 | 0.84 | 0.76 | 0.65 | 63 |
| 226841_at | macrophage expressed gene 1 | MPEG1 | BF590697 | 1.06 | 1.62 | 1.76 | 87 |
| 214048_at | methyl-CpG binding domain | MBD4 | AI913365 | 1.03 | 0.96 | 0.65 | 89 |
| | protein 4 | | | | | | |
| 239001_at | microsomal glutathione S- | MGST1 | AV705233 | 1.19 | 1.33 | 1.40 | 62 |
| | transferase 1 | | | | | | |
| 217980_s_at | mitochondrial ribosomal | MRPL16 | NM_017840 | 0.82 | 0.84 | 0.65 | 609 |
| | protein L16 | | | | | | |
| 231274_s_at | mitochondrial solute carrier | MSCP | R92925 | 0.79 | 0.81 | 0.69 | 193 |
| | protein | | | | | | |
| 1558732_at | mitogen-activated protein | MAP4K4 | AK074900 | 0.82 | 0.87 | 0.70 | 128 |
| | kinase kinase kinase kinase 4 | | | | | | |
| 223218_s_at | molecule possessing ankyrin | MAIL | AB037925 | 0.84 | 0.75 | 0.71 | 708 |
| | repeats induced by | | | | | | |
| | lipopolysaccharide (MAIL), | | | | | | |
| | homolog of mouse | | | | | | |
| 1563469_at | MRNA; cDNA | | AL832681 | 1.35 | 1.30 | 1.38 | 74 |
| | DKFZp313M0417 (from | | | | | | |
| | clone DKFZp313M0417) | | | | | | |
| 234224_at | MRNA; cDNA | | AL137541 | 0.93 | 0.79 | 0.80 | 79 |
| | DKFZp434O0919 (from | | | | | | |
| | clone DKFZp434O0919) | | | | | | |
| 227576_at | MRNA; cDNA | | AW003140 | 0.99 | 0.77 | 0.69 | 452 |
| | DKFZp686K1098 (from | | | | | | |
| | clone DKFZp686K1098) | | | | | | |
| 228217_s_at | MRNA; cDNA | | BF973374 | 1.02 | 1.41 | 1.77 | 365 |
| | DKFZp686P09209 (from | | | | | | |
| | clone DKFZp686P09209) | | | | | | |
| 210210_at | myelin protein zero-like 1 | MPZL1 | AF181660 | 1.24 | 1.41 | 1.78 | 105 |
| 233539_at | N-acyl- | NAPE-PLD | AK000801 | 1.15 | 1.37 | 1.69 | 135 |
| | phosphatidylethanolamine- | | | | | | |
| | hydrolyzing phospholipase D | | | | | | |
| 202000_at | NADH dehydrogenase | NDUFA6 | BC002772 | 1.20 | 1.48 | 1.45 | 693 |
| | (ubiquinone) 1 alpha | | | | | | |
| | subcomplex, 6, 14 kDa | | | | | | |
| 218320_s_at | neuronal protein 17.3 | P17.3 | NM_019056 | 0.87 | 0.67 | 0.68 | 993 |
| 233626_at | neuropilin 1 | NRP1 | AK024580 | 1.38 | 1.39 | 1.43 | 53 |
| 235985_at | nj45a06.x5 NCI_CGAP_Pr9 | | AI821477 | 0.96 | 0.80 | 0.73 | 115 |
| | Homo sapiens cDNA clone | | | | | | |
| | IMAGE: 995410 similar to | | | | | | |
| | contains Alu repetitive | | | | | | |
| | element; contains element | | | | | | |
| | TAR1 repetitive element;, | | | | | | |
| | mRNA sequence. | | | | | | |
| 226991_at | nuclear factor of activated T- | | AA489681 | 1.38 | 1.73 | 1.87 | 88 |
| | cells, cytoplsamic, | | | | | | |
| | calcineurin-dependent 2 | | | | | | |
| 206302_s_at | nudix (nucleoside diphosphate | NUDT4 | NM_019094 | 1.29 | 1.35 | 1.52 | 955 |
| | linked moiety X)-type motif 4 | | | | | | |
| 238408_at | oxidation resistance 1 | OXR1 | AW086258 | 1.27 | 1.28 | 1.46 | 84 |
| 205336_at | parvalbumin | PVALB | NM_002854 | 0.87 | 0.71 | 0.74 | 319 |
| 204300_at | PET112-like (yeast) | PET112L | NM_004564 | 1.21 | 1.39 | 1.55 | 205 |
| 209504_s_at | pleckstrin homology domain | PLEKHB1 | AF081583 | 1.34 | 1.59 | 1.55 | 144 |
| | containing, family B | | | | | | |
| | (evectins) member 1 | | | | | | |
| 242922_at | pM5 protein | PM5 | AU151198 | 1.21 | 1.23 | 1.49 | 60 |
| 236407_at | potassium voltage-gated | KCNE1 | R73518 | 1.28 | 1.47 | 1.52 | 127 |
| | channel, Isk-relatad family, | | | | | | |
| | member 1 | | | | | | |
| 1568706_s_at | Pp12719 mRNA, complete | | AF318328 | 1.38 | 1.42 | 2.03 | 96 |
| | cds | | | | | | |
| 1558017_s_at | PRKC, apoptosis, WT1, | PAWR | BG109597 | 1.24 | 1.37 | 1.47 | 179 |
| | regulator | | | | | | |
| 200979_at | pyruvate dehydrogenase | PDHA1 | BF739979 | 1.29 | 1.49 | 1.69 | 650 |
| | (lipoamide) alpha 1 | | | | | | |
| 223802_s_at | retinoblastoma binding | RBBP6 | AF063596 | 1.43 | 1.69 | 1.97 | 249 |
| | protein 6 | | | | | | |
| 225171_at | Rho GTPase activating | ARHGAP18 | BE644830 | 1.16 | 1.28 | 1.47 | 1407 |
| | protein 18 | | | | | | |
| 221989_at | ribosomal protein L10 | RPL10 | AW057781 | 1.11 | 1.35 | 1.69 | 212 |
| 1555878_at | ribosomal protein S24 | RPS24 | AK094613 | 1.63 | 1.79 | 1.66 | 138 |
| 212030_at | RNA-binding region (RNP1, | RNPC7 | BG251218 | 1.11 | 1.42 | 1.74 | 293 |
| | RRM) containing 7 | | | | | | |
| 241996_at | RUN and FYVE domain | RUFY2 | AI669591 | 1.52 | 1.92 | 1.44 | 194 |
| | containing 2 | | | | | | |
| 215028_at | sema domain, transmembrane | SEMA6A | AB002438 | 1.05 | 1.43 | 1.30 | 63 |
| | domain (TM), and | | | | | | |
| | cytoplasmic domain, | | | | | | |
| | (semaphorin) 6A | | | | | | |
| 1559263_s_at | Similar to hypothetical protein | | BG397809 | 1.34 | 1.37 | 1.54 | 96 |
| | D730019B10 (LOC340152), | | | | | | |
| | mRNA | | | | | | |
| 222145_at | Similar to PI-3-kinase-related | | AK027225 | 1.16 | 1.15 | 1.34 | 64 |
| | kinase SMG-1 isoform 1; | | | | | | |
| | lambda/iota protein kinase C- | | | | | | |
| | interacting protein; | | | | | | |
| | phosphatidylinositol 3-kinase- | | | | | | |
| | related protein kinase | | | | | | |
| | (LOC390682), mRNA | | | | | | |
| 202781_s_at | skeletal muscle and kidney | SKIP | AI806031 | 0.79 | 0.79 | 0.64 | 101 |
| | enriched inositol phosphatase | | | | | | |
| 217591_at | SKI-like | SKIL | BF725121 | 1.21 | 1.07 | 1.63 | 114 |
| 1559351_at | solute carrier family 16 | SLC16A9 | BI668873 | 1.67 | 1.36 | 1.80 | 138 |
| | (monocarboxylic acid | | | | | | |
| | transporters), member 9 | | | | | | |
| 244353_s_at | solute carrier family 2 | SLC2A12 | AI675682 | 1.09 | 1.21 | 1.74 | 125 |
| | (facilitated glucose | | | | | | |
| | transporter), member 12 | | | | | | |
| 231437_at | solute carrier family 35, | SLC35D2 | AA693722 | 1.81 | 1.71 | 1.87 | 120 |
| | member D2 | | | | | | |
| 233123_at | solute carrier family 40 (iron- | SLC40A1 | AU156956 | 1.43 | 1.85 | 2.09 | 120 |
| | regulated transporter), | | | | | | |
| | member 1 | | | | | | |
| 232392_at | splicing factor, | SFRS3 | BE927772 | 1.39 | 1.64 | 1.60 | 565 |
| | arginine/serine-rich 3 | | | | | | |
| 204690_at | syntaxin 8 | STX8 | NM_004853 | 1.00 | 1.19 | 1.48 | 622 |
| 221617_at | TAF9-like RNA polymerase | | AF077053 | 1.22 | 1.37 | 1.75 | 80 |
| | II, TATA box binding protein | | | | | | |
| | (TBP)-associated factor, | | | | | | |
| | 31 kDa | | | | | | |
| 221938_x_at | thyroid hormone receptor | THRAP5 | AW262690 | 1.18 | 1.11 | 1.73 | 168 |
| | associated protein 5 | | | | | | |
| 228793_at | thyroid hormone receptor | TRIP8 | BF002296 | 1.43 | 1.60 | 1.92 | 395 |
| | interactor 8 | | | | | | |
| 210886_x_at | TP53 activated protein 1 | TP53AP1 | AB007457 | 1.33 | 1.37 | 2.04 | 182 |
| 228971_at | Transcribed sequence with | | AI357655 | 1.07 | 1.19 | 1.46 | 704 |
| | moderate similarity to protein | | | | | | |
| | ref: NP_055301.1 (H. sapiens) | | | | | | |
| | neuronal thread protein | | | | | | |
| | [Homo sapiens] | | | | | | |
| 233518_at | Transcribed sequence with | | AU144449 | 0.97 | 1.20 | 1.57 | 74 |
| | moderate similarity to protein | | | | | | |
| | ref: NP_071431.1 (H. sapiens) | | | | | | |
| | cytokine receptor-like factor | | | | | | |
| | 2; cytokine receptor CRL2 | | | | | | |
| | precusor [Homo sapiens] | | | | | | |
| 241798_at | Transcribed sequence with | | AI339930 | 0.77 | 0.64 | 0.73 | 69 |
| | moderate similarity to protein | | | | | | |
| | sp: P39195 (H. sapiens) | | | | | | |
| | ALU8_HUMAN Alu | | | | | | |
| | subfamily SX sequence | | | | | | |
| | contamination warning entry | | | | | | |
| 243256_at | Transcribed sequence with | | AW796364 | 1.31 | 1.47 | 1.54 | 157 |
| | weak similarity to protein | | | | | | |
| | ref: NP_060265.1 (H. sapiens) | | | | | | |
| | hypothetical protein | | | | | | |
| | FLJ20378 [Homo sapiens] | | | | | | |
| 239735_at | Transcribed sequence with | | N67106 | 1.33 | 1.27 | 1.56 | 150 |
| | weak similarity to protein | | | | | | |
| | ref: NP_060312.1 (H. sapiens) | | | | | | |
| | hypothetical protein | | | | | | |
| | FLJ20489 [Homo sapiens] | | | | | | |
| 242191_at | Transcribed sequence with | | AI701905 | 0.68 | 0.50 | 0.49 | 174 |
| | weak similarity to protein | | | | | | |
| | ref: NP_060312.1 (H. sapiens) | | | | | | |
| | hypothetical protein | | | | | | |
| | FLJ20489 [Homo sapiens] | | | | | | |
| 242490_at | Transcribed sequence with | | AA564255 | 1.16 | 1.23 | 1.55 | 165 |
| | weak similarity to protein | | | | | | |
| | ref: NP_062553.1 (H. sapiens) | | | | | | |
| | hypothetical protein | | | | | | |
| | FLJ11267 [Homo sapiens] | | | | | | |
| 241897_at | Transcribed sequence with | | AA491949 | 1.32 | 1.49 | 1.93 | 492 |
| | weak similarity to protein | | | | | | |
| | ref: NP_071431.1 (H. sapiens) | | | | | | |
| | cytokine receptor-like factor | | | | | | |
| | 2; cytokine receptor CRL2 | | | | | | |
| | precusor [Homo sapiens] | | | | | | |
| 230590_at | Transcribed sequences | | BE675486 | 0.88 | 0.81 | 0.67 | 107 |
| 230733_at | Transcribed sequences | | H98113 | 0.67 | 0.63 | 0.61 | 127 |
| 230773_at | Transcribed sequences | | AA628511 | 1.09 | 1.26 | 1.60 | 131 |
| 237317_at | Transcribed sequences | | AW136338 | 1.02 | 0.75 | 0.70 | 79 |
| 239238_at | Transcribed sequences | | AI208857 | 1.35 | 2.25 | 2.19 | 113 |
| 240128_at | Transcribed sequences | | H94876 | 1.18 | 1.34 | 1.62 | 54 |
| 241837_at | Transcribed sequences | | AI289774 | 1.64 | 1.71 | 1.73 | 59 |
| 241936_x_at | Transcribed sequences | | AI654130 | 1.07 | 1.17 | 1.51 | 175 |
| 241940_at | Transcribed sequences | | BF477544 | 1.22 | 1.25 | 1.66 | 63 |
| 242299_at | Transcribed sequences | | AW274468 | 0.80 | 0.77 | 0.70 | 82 |
| 242536_at | Transcribed sequences | | AI522220 | 1.25 | 1.28 | 1.97 | 533 |
| 242579_at | Transcribed sequences | | AA935461 | 1.35 | 1.16 | 1.73 | 270 |
| 242673_at | Transcribed sequences | | AA931284 | 1.36 | 1.55 | 1.62 | 99 |
| 243591_at | Transcribed sequences | | AI887749 | 1.30 | 1.72 | 2.12 | 106 |
| 243675_at | Transcribed sequences | | BF512500 | 1.12 | 1.42 | 1.89 | 81 |
| 243933_at | Transcribed sequences | | AI096634 | 1.15 | 1.24 | 1.48 | 142 |
| 244414_at | Transcribed sequences | | AI148006 | 1.31 | 1.62 | 1.54 | 439 |
| 244674_at | Transcribed sequences | | AA936428 | 1.19 | 1.11 | 1.54 | 131 |
| 244797_at | Transcribed sequences | | AI269245 | 1.37 | 1.23 | 1.57 | 168 |
| 224566_at | trophoblast-derived | TncRNA | AI042152 | 1.27 | 1.42 | 1.95 | 1769 |
| | noncoding RNA | | | | | | |
| 202510_s_at | tumor necrosis factor, alpha- | TNFAIP2 | NM_006291 | 1.46 | 1.63 | 1.71 | 211 |
| | induced protein 2 | | | | | | |
| 232141_at | U2(RNU2) small nuclear | U2AF1 | AU144161 | 1.03 | 1.24 | 1.32 | 109 |
| | RNA auxiliary factor 1 | | | | | | |
| 228142_at | ubiquinol-cytochrome c | HSPC051 | BE208777 | 1.34 | 1.39 | 1.42 | 177 |
| | reductase complex (7.2 kD) | | | | | | |
| 1557409_at | UI-CF-FN0-aex-p-22-0-UI.s1 | | CA313226 | 1.19 | 1.56 | 1.64 | 124 |
| | UI-CF-FN0 Homo sapiens | | | | | | |
| | cDNA clone UI-CF-FN0-aex- | | | | | | |
| | p-22-0-UI 3′, mRNA | | | | | | |
| | sequence. | | | | | | |
| 1558801_at | unnamed protein product; | | AK055769 | 1.14 | 1.40 | 1.55 | 169 |
| | Homo sapiens cDNA | | | | | | |
| | FLJ31207 fis, clone | | | | | | |
| | KIDNE2003357. | | | | | | |
| 225198_at | VAMP (vesicle-aaaociated | VAPA | AL571942 | 1.78 | 1.90 | 2.34 | 658 |
| | membrane protein)-associated | | | | | | |
| | protein A, 33 kDa | | | | | | |
| 222303_at | v-ets erythroblastosis virus | ETS2 | AV700891 | 1.32 | 1.86 | 2.52 | 177 |
| | E26 oncogene homolog 2 | | | | | | |
| | (avian) | | | | | | |
| 235850_at | WD repeat domain 5B | WDR5B | BF434228 | 1.13 | 1.21 | 1.56 | 289 |
| 229647_at | wh65e08.x1 | | AI762401 | 2.01 | 2.01 | 2.22 | 793 |
| | NCI_CGAP_Kid11 Homo | | | | | | |
| | sapiens cDNA clone | | | | | | |
| | IMAGE: 2385638 3′ similar to | | | | | | |
| | contains Alu repetitive | | | | | | |
| | element; contains element | | | | | | |
| | MER22 repetitive element;, | | | | | | |
| | mRNA sequence. | | | | | | |
| 242406_at | wl47a04.x1 NCI_CGAP_Ut1 | | AI870547 | 0.73 | 0.58 | 0.70 | 126 |
| | Homo sapiens cDNA clone | | | | | | |
| | IMAGE: 2428014 3′, mRNA | | | | | | |
| | sequence. | | | | | | |
| 224590_at | X (inactive)-specific transcript | XIST | BE644917 | 1.26 | 1.44 | 1.54 | 261 |
| 238913_at | xm54d01.x1 | | AW235215 | 1.25 | 1.60 | 1.64 | 111 |
| | NCI_CGAP_GC6 Homo | | | | | | |
| | sapiens cDNA clone | | | | | | |
| | IMAGE: 2688001 3′ similar to | | | | | | |
| | contains Alu repetitive | | | | | | |
| | element; contains element | | | | | | |
| | MER28 MER28 repetitive | | | | | | |
| | element;, mRNA sequence. | | | | | | |
| 222281_s_at | xs86h03.x1 NCI_CGAP_Ut2 | | AW517716 | 1.47 | 1.56 | 1.78 | 350 |
| | Homo sapiens cDNA clone | | | | | | |
| | IMAGE: 2776565 3′ similar to | | | | | | |
| | contains Alu repetitive | | | | | | |
| | element; contains element | | | | | | |
| | MER38 repetitive element;, | | | | | | |
| | mRNA sequence. | | | | | | |
| 234033_at | yd35c06.s1 Soares fetal liver | | T71269 | 1.15 | 1.19 | 1.61 | 130 |
| | spleen 1NFLS Homo sapiens | | | | | | |
| | cDNA clone IMAGE: 110218 | | | | | | |
| | 3′, mRNA sequence. | | | | | | |
| 239654_at | ye62h04.s1 Soares fetal liver | | T98846 | 1.07 | 1.32 | 1.62 | 139 |
| | spleen 1NFLS Homo sapiens | | | | | | |
| | cDNA clone IMAGE: 122359 | | | | | | |
| | 3′, mRNA sequence. | | | | | | |
| 242241_x_at | yi33f06.s1 Soares placenta | | R66713 | 114 | 1.36 | 1.63 | 73 |
| | Nb2HP Homo sapiens cDNA | | | | | | |
| | clone IMAGE: 141059 3′ | | | | | | |
| | similar to contains Alu | | | | | | |
| | repetitive element; contains L1 | | | | | | |
| | repetitive element;, mRNA | | | | | | |
| | sequence. | | | | | | |
| 1565566_a_at | yn76g07.s1 Soares adult brain | | H21394 | 0.96 | 1.26 | 1.35 | 84 |
| | N2b5HB55Y Homo sapiens | | | | | | |
| | cDNA clone IMAGE: 174396 | | | | | | |
| | 3′ similar to contains Alu | | | | | | |
| | repetitive element;, mRNA | | | | | | |
| | sequence. | | | | | | |
| 217586_x_at | yy28g05.s1 Soares | | N35922 | 1.44 | 1.53 | 1.58 | 370 |
| | melanocyte 2NbHM Homo | | | | | | |
| | sapiens cDNA clone | | | | | | |
| | IMAGE: 272600 3′ similar to | | | | | | |
| | contains Alu repetitive | | | | | | |
| | element;, mRNA sequence. | | | | | | |
| 226163_at | zinc finger and BTB domain | ZBTB9 | AW291499 | 1.27 | 1.15 | 1.56 | 159 |
| | containing 9 | | | | | | |
| 1569312_at | zinc finger protein 146 | ZNF146 | BE383308 | 1.08 | 1.24 | 1.53 | 85 |
| 231848_x_at | zinc finger protein 207 | ZNF207 | AW192569 | 0.94 | 0.56 | 0.66 | 344 |
| 239937_at | zinc finger protein 207 | ZNF207 | AI860558 | 1.02 | 1.17 | 1.52 | 128 |
| 215012_at | zinc finger protein 451 | ZNF451 | AU144775 | 1.35 | 2.08 | 2.60 | 153 |
| 219741_x_at | zinc finger protein 552 | ZNF552 | NM_024762 | 1.20 | 1.31 | 1.66 | 184 |
| 230503_at | zo02d03.s1 Stratagene colon | | AA151917 | 0.69 | 0.68 | 0.68 | 159 |
| | (#937204) Homo sapiens | | | | | | |
| | cDNA clone IMAGE: 566501 | | | | | | |
| | 3′, mRNA sequence. | | | | | | |

3.3.4 Biomarker Global Analysis; OPLS Model, Result

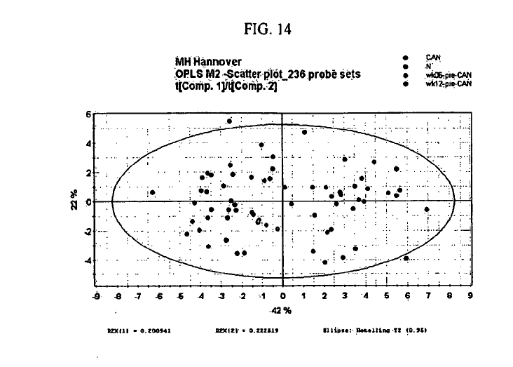

FIG. 14 shows the Biomarker global analysis OPLS model: Scatter plot. A scatterplot or scatter graph is a graph used in statistics to visually display and compare two sets of related quantitative, or numerical, data by displaying only finitely many points, each having a coordinate on a horizontal and a vertical axis. In FIG. 2 each dot represents a sample of a patient. Relative distance between data points is a measure of relationship/resemblance. The separation of the “N” samples from the “week 06 pre-CAN”, “week 12 pre-CAN”, “CAN” samples indicates the potency of the algorithm /model to discriminate between the data points with the use of these probe sets.

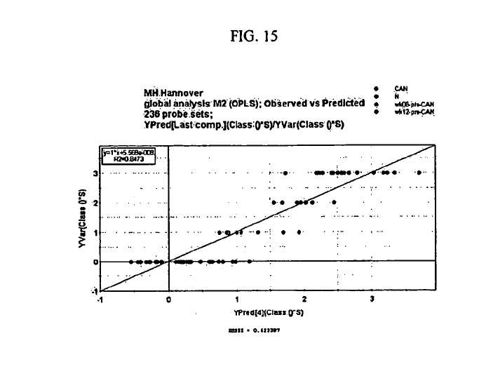

FIG. 15 shows the Biomarker global analysis OPLS model: observed vs prediction.

Validation by response permutation is an internal cross-validation, which creates a training set and a test set of samples. A model is fitted to explain the test set based on the training set and the values for R²Y (explained variance) and Q² (predicted variance) are computed and plotted. By random permutation of the training and test sets, a number of R²Y/Q² are obtained. The validate plot is then created by letting the Y-axis represent the R²Y/Q²-values of all models, including the “real” one, and by assigning the X-axis to the correlation coefficients between permuted and original response variables. A regression line is then fitted among the R²Y points and another one through the Q² points. The intercepts of the regression lines are interpretable as measures of “background” R2Y and Q² obtained to fit the data. Intercepts around 0.4 and below for R2Y and around 0.05 and below for Q² indicate valid models. Since these criteria are met in this model it is an indication of a valid model for the present dataset.

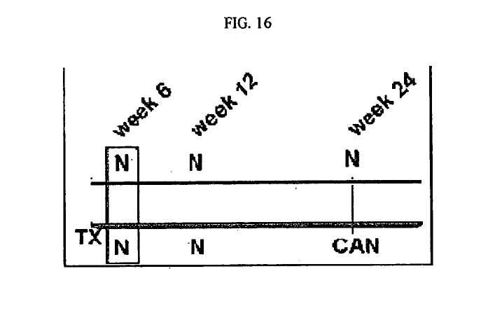

FIG. 16 shows the Biomarker global analysis OPLS model: observed vs predicted.

The prediction of the Y space samples can be plotted as a scatter plot. RMSE (Root mean square error) is the standard deviation of the predicted residuals (error), and is computed as the square root of (Σ(obs-pred)²/N). A small RMSE is a measure for a good fit of a model.

The Y-axis of the plot represents the observed classes of the model, the X-axis the predicted classes. A match of Y- and X-values in this plot demonstrates the good fit of the model.

The combination of biomarker genes that form a molecular signature after tissue transplantation as determined by global data analysis using OPLS model are shown in Table 11.

TABLE 11
Genes of the Biomarker Global Analysis, OPLS Model

| | | | | Fold | Fold | | Stable |
|---|---|---|---|---|---|---|---|
| | | | | change | change | | Graft: |
| | | | | wk06- | wk12- | Fold | Raw |
| Affymetrix | | | | pre- | pre- | change | Expression |
| Probe Set ID | Description | Common | Genbank | CAN | CAN | CAN | Value |
| 244567_at | 602343781F1 NIH_MGC_89 | | BG165613 | 1.5 | 1.2 | 1.7 | 103 |
| | Homo sapiens cDNA clone | | | | | | |
| | IMAGE: 4453556 5′, mRNA | | | | | | |
| | sequence. | | | | | | |
| 244145_at | 602371458F1 NIH_MGC_93 | | BG260337 | 1.2 | 2.0 | 1.7 | 102 |
| | Homo sapiens cDNA clone | | | | | | |
| | IMAGE: 4479327 5′, mRNA | | | | | | |
| | sequence. | | | | | | |
| 232175_at | ADP-ribosylation factor 1 | ARF1 | AI972094 | 1.5 | 1.6 | 1.5 | 108 |
| 238996_x_at | aldolase A, fructose- | ALDOA | AI921586 | 1.9 | 2.3 | 1.9 | 413 |
| | bisphosphate | | | | | | |
| 232865_at | ALL1 fused gene from 5q31 | AF5Q31 | N59653 | 1.4 | 1.6 | 1.8 | 179 |
| 236778_at | alpha thalassemia/mental | ATRX | AA826176 | 1.6 | 1.5 | 2.0 | 77 |
| | retardation syndrome X- | | | | | | |
| | linked (RAD54 homolog, S. cerevisiae) | | | | | | |
| 1563792_at | amnionless homolog (mouse) | AMN | AK092824 | 1.1 | 1.2 | 1.9 | 98 |
| 226718_at | amphoterin-induced gene | KIAA1163 | AA001423 | 1.4 | 1.6 | 1.8 | 142 |
| 229903_x_at | amylase, alpha 2B; pancreatic | AMY2B | AI632212 | 1.1 | 1.2 | 1.4 | 350 |
| 219962_at | angiotensin I converting | ACE2 | NM_021804 | 1.3 | 1.6 | 1.5 | 378 |
| | enzyme (peptidyl-dipeptidase | | | | | | |
| | A) 2 | | | | | | |
| 227260_at | ankyrin repeat domain 10 | ANKRD10 | AV724266 | 1.2 | 1.3 | 1.7 | 708 |
| 230972_at | ankyrin repeat domain 9 | ANKRD9 | AW194999 | 1.3 | 1.5 | 1.5 | 656 |
| 224489_at | ARF protein | LOC51326 | BC006271 | 0.8 | 0.6 | 0.6 | 86 |
| 206993_at | ATP synthase, H+ | ATP5S | NM_015684 | 1.2 | 2.0 | 1.5 | 119 |
| | transporting, mitochondrial F0 | | | | | | |
| | complex, subunit s (factor B) | | | | | | |
| 204719_at | ATP-binding cassette, sub- | ABCA8 | NM_007168 | 2.0 | 2.1 | 3.4 | 350 |
| | family A (ABC1), member 8 | | | | | | |
| 233271_at | AU145563 HEMBA1 Homo | | AU145563 | 0.8 | 0.5 | 0.7 | 143 |
| | sapiens cDNA clone | | | | | | |
| | HEMBA1005133 3′, mRNA | | | | | | |
| | sequence. | | | | | | |
| 215204_at | AU147295 MAMMA1 Homo | | AU147295 | 1.3 | 1.2 | 1.5 | 90 |
| | sapiens cDNA clone | | | | | | |
| | MAMMA1000264 3′, mRNA | | | | | | |
| | sequence. | | | | | | |
| 236892_s_at | B1 for mucin | HAB1 | BF590528 | 1.3 | 1.3 | 1.6 | 312 |
| 239791_at | B1 for mucin | HAB1 | AI125255 | 1.0 | 1.0 | 0.7 | 94 |
| 227896_at | BRCA2 and CDKN1A | BCCIP | AI373643 | 1.6 | 1.7 | 1.6 | 223 |
| | interacting protein | | | | | | |
| 223679_at | catenin (cadherin-associated | CTNNB1 | AF130085 | 1.2 | 1.3 | 1.6 | 146 |
| | protein), beta 1, 88 kDa | | | | | | |
| 233019_at | CCR4-NOT transcription | CNOT7 | AU145061 | 1.6 | 1.7 | 2.0 | 89 |
| | complex, subunit 7 | | | | | | |
| 204510_at | CDC7 cell division cycle 7 (S. cerevisiae) | CDC7 | NM_003503 | 1.5 | 1.8 | 1.7 | 104 |
| 233399_x_at | CDNA clone | | AU145662 | 1.3 | 1.1 | 1.8 | 183 |
| | IMAGE: 30352956, partial cds | | | | | | |
| 232351_at | CDNA FLJ10150 fis, clone | | AK022308 | 0.9 | 0.8 | 0.7 | 152 |
| | HEMBA1003395 | | | | | | |
| 234074_at | CDNA FLJ10946 fis, clone | | AU155494 | 1.4 | 1.3 | 1.4 | 99 |
| | PLACE1000005 | | | | | | |
| 227140_at | CDNA FLJ11041 fis, clone | | AI343467 | 0.8 | 0.7 | 0.7 | 108 |
| | PLACE1004405 | | | | | | |
| 232544_at | CDNA FLJ11572 fis, clone | | AU144916 | 1.4 | 1.6 | 1.4 | 231 |
| | HEMBA1003373 | | | | | | |
| 232991_at | CDNA FLJ11613 fis, clone | | AK021675 | 0.9 | 0.8 | 0.7 | 107 |
| | HEMBA1004012 | | | | | | |
| 232952_at | CDNA FLJ11942 fis, clone | | AU146493 | 0.6 | 0.6 | 0.7 | 83 |
| | HEMBB1000652 | | | | | | |
| 230791_at | CDNA FLJ12033 fis, clone | | AU146924 | 1.0 | 0.9 | 0.7 | 241 |
| | HEMBB1001899 | | | | | | |
| 233498_at | CDNA FLJ14142 fis, clone | | AK024204 | 0.9 | 0.8 | 0.7 | 282 |
| | MAMMA1002880 | | | | | | |
| 230986_at | CDNA FLJ30065 fis, clone | | AI821447 | 1.1 | 1.4 | 1.3 | 96 |
| | ADRGL2000328 | | | | | | |
| 241941_at | CDNA FLJ31511 fis, clone | | AA778747 | 0.9 | 0.8 | 0.7 | 75 |
| | NT2RI1000035 | | | | | | |
| 1557270_at | CDNA FLJ36375 fis, clone | | AA632049 | 1.2 | 1.5 | 1.7 | 283 |
| | THYMU2008226 | | | | | | |
| 235028_at | CDNA FLJ46440 fis, clone | | BG288330 | 1.5 | 2.1 | 1.5 | 659 |
| | THYMU3016022 | | | | | | |
| 234604_at | CDNA: FLJ21228 fis, clone | | AK024881 | 1.6 | 2.0 | 1.6 | 62 |
| | COL00739 | | | | | | |
| 233824_at | CDNA: FLJ21428 fis, clone | | AK025081 | 0.8 | 0.7 | 0.5 | 114 |
| | COL04203 | | | | | | |
| 216782_at | CDNA: FLJ23026 fis, clone | | AK026679 | 0.7 | 0.7 | 0.6 | 488 |
| | LNG01738 | | | | | | |
| 214196_s_at | ceroid-lipofuscinosis, | CLN2 | AA602532 | 1.6 | 1.3 | 1.9 | 84 |
| | neuronal 2, late infantile | | | | | | |
| | (Jansky-Bielschowsky | | | | | | |
| | disease) | | | | | | |
| 228143_at | ceruloplasmin (ferroxidase) | CP | AI684991 | 0.9 | 0.8 | 0.8 | 69 |
| 223191_at | chromosome 14 open reading | C14orf112 | AF151037 | 0.7 | 0.7 | 0.7 | 541 |
| | frame 112 | | | | | | |
| 218796_at | chromosome 20 open reading | C20orf42 | NM_017671 | 1.4 | 5.8 | 3.9 | 107 |
| | frame 42 | | | | | | |
| 218453_s_at | chromosome 6 open reading | C6orf35 | NM_018452 | 0.7 | 0.7 | 0.6 | 110 |
| | frame 35 | | | | | | |
| 229012_at | chromosome 9 open reading | C9orf24 | AW269443 | 1.2 | 1.6 | 1.4 | 142 |
| | frame 24 | | | | | | |
| 1552455_at | chromosome 9 open reading | C9orf65 | NM_138818 | 1.6 | 2.0 | 1.6 | 81 |
| | frame 65 | | | | | | |
| 225377_at | chromosome 9 open reading | C9orf86 | BE783949 | 0.8 | 0.6 | 0.4 | 173 |
| | frame 86 | | | | | | |
| 239683_at | citrate lyase beta like | CLYBL | AI476268 | 1.2 | 1.3 | 1.5 | 243 |
| 215504_x_at | Clone 25061 mRNA sequence | | AF131777 | 0.7 | 0.6 | 0.7 | 482 |
| 243329_at | Clone IMAGE: 121662 | | AI074450 | 1.0 | 1.0 | 0.7 | 195 |
| | mRNA sequence | | | | | | |
| 231808_at | Clone IMAGE: 5302006, | | AY007106 | 1.0 | 1.2 | 1.4 | 213 |
| | mRNA | | | | | | |
| 205229_s_at | coagulation factor C homolog, | COCH | AA669336 | 0.8 | 1.5 | 1.5 | 86 |
| | cochlin (Limulus | | | | | | |
| | polyphemus) | | | | | | |
| 225288_at | collagen, type XXVII, alpha 1 | COL27A1 | AI949136 | 1.3 | 1.7 | 1.6 | 304 |
| 205159_at | colony stimulating factor 2 | CSF2RB | AV756141 | 1.0 | 1.5 | 1.4 | 106 |
| | receptor, beta, low-affinity | | | | | | |
| | (granulocyte-macrophage) | | | | | | |
| 211025_x_at | cytochrome c oxidase subunit | COX5B | BC006229 | 1.4 | 1.5 | 1.4 | 1299 |
| | Vb | | | | | | |
| 225503_at | dehydrogenase/reductase | DHRSX | AL547782 | 1.1 | 1.4 | 1.5 | 178 |
| | (SDR family) X-linked | | | | | | |
| 1556820_a_at | deleted in lymphocytic | DLEU2 | H48516 | 1.3 | 1.1 | 1.5 | 67 |
| | leukemia, 2 | | | | | | |
| 1556821_x_at | deleted in lymphocytic | DLEU2 | H48516 | 1.4 | 1.4 | 1.8 | 100 |
| | leukemia, 2 | | | | | | |
| 210165_at | deoxyribonuclease 1 | DNASE1 | M55983 | 1.3 | 1.3 | 1.6 | 149 |
| 218650_at | DiGeorge syndrome critical | DGCR8 | NM_022775 | 1.2 | 1.2 | 1.6 | 167 |
| | region gene 8 | | | | | | |
| 223763_at | dystrobrevin binding protein 1 | DTNBP1 | AL136637 | 1.4 | 1.6 | 1.6 | 82 |
| 227353_at | epidermodysplasia | EVER2 | BE671663 | 1.1 | 1.2 | 1.4 | 85 |
| | verruciformis 2 | | | | | | |
| 236520_at | EST384471 MAGE | | AW972380 | 1.4 | 1.6 | 2.2 | 128 |
| | resequences, MAGL Homo | | | | | | |
| | sapiens cDNA, mRNA | | | | | | |
| | sequence. | | | | | | |
| 214805_at | eukaryotic translation | EIF4A1 | U79273 | 1.5 | 1.4 | 1.7 | 153 |
| | initiation factor 4A, isoform 1 | | | | | | |
| 230389_at | formin binding protein 1 | FNBP1 | BE046511 | 1.2 | 1.2 | 1.7 | 188 |
| 244509_at | G protein-coupled receptor | GPR155 | AW449728 | 1.2 | 1.2 | 1.6 | 69 |
| | 155 | | | | | | |
| 210358_x_at | GATA binding protein 2 | GATA2 | BC002557 | 0.9 | 07 | 0.8 | 111 |
| 227163_at | glutathione S-transferase | GSTO2 | AL162742 | 0.9 | 0.7 | 0.7 | 361 |
| | omega 2 | | | | | | |
| 215203_at | golgi autoantigen, golgin | GOLGA4 | AW438464 | 0.9 | 0.8 | 0.7 | 109 |
| | subfamily a, 4 | | | | | | |
| 229255_x_at | golgi SNAP receptor complex | GOSR2 | BF593917 | 0.7 | 0.7 | 0.7 | 142 |
| | member 2 | | | | | | |
| 240405_at | H326 | H326 | AA707411 | 1.3 | 1.4 | 1.4 | 61 |
| 203394_s_at | hairy and enhancer of split 1, | HES1 | BE973687 | 1.9 | 2.2 | 1.8 | 703 |
| | (Drosophila) | | | | | | |
| 209960_at | hepatocyte growth factor | HGF | X16323 | 0.8 | 0.8 | 0.7 | 118 |
| | (hepapoietin A; scatter factor) | | | | | | |
| 213359_at | heterogeneous nuclear | HNRPD | W74620 | 0.8 | 0.7 | 0.6 | 207 |
| | ribonucleoprotein D (AU-rich | | | | | | |
| | element RNA binding protein | | | | | | |
| | 1, 37 kDa) | | | | | | |
| 1560782_at | Homo sapiens cDNA clone | | BC035326 | 1.7 | 1.6 | 1.9 | 101 |
| | IMAGE: 5186324, partial cds. | | | | | | |
| 215553_x_at | Homo sapiens cDNA | | AK024315 | 1.5 | 1.6 | 2.0 | 262 |
| | FLJ14253 fis, clone | | | | | | |
| | OVARC1001376. | | | | | | |
| 233813_at | Homo sapiens cDNA: | | AK026900 | 1.7 | 1.4 | 1.9 | 76 |
| | FLJ23247 fis, clone | | | | | | |
| | COL03425. | | | | | | |
| 231886_at | Homo sapiens mRNA; cDNA | | AL137655 | 0.8 | 0.8 | 0.7 | 73 |
| | DKFZp434B2016 (from clone | | | | | | |
| | DKFZp434B2016). | | | | | | |
| 228564_at | hypothetical gene supported | | AI569804 | 1.3 | 1.5 | 1.5 | 439 |
| | by BC013438 | | | | | | |
| 241031_at | hypothetical LOC145741 | | BE218239 | 1.5 | 1.7 | 2.0 | 68 |
| 237108_x_at | hypothetical protein | DKFZp761G0122 | AW611845 | 1.0 | 1.3 | 1.7 | 276 |
| | DKFZp761G0122 | | | | | | |
| 219074_at | hypothetical protein | FLJ10846 | NM_018241 | 1.1 | 1.2 | 1.6 | 418 |
| | FLJ10846 | | | | | | |
| 222788_s_at | hypothetical protein | FLJ11220 | BE888593 | 0.9 | 0.8 | 0.6 | 106 |
| | FLJ11220 | | | | | | |
| 226967_at | hypothetical protein | FLJ14768 | BG231981 | 1.6 | 2.1 | 1.4 | 156 |
| | FLJ14768 | | | | | | |
| 1557828_a_at | hypothetical protein | FLJ21657 | BE675061 | 0.8 | 0.8 | 0.7 | 148 |
| | FLJ21657 | | | | | | |
| 222872_x_at | hypothetical protein | FLJ22833 | AU157541 | 1.4 | 1.5 | 1.6 | 456 |
| | FLJ22833 | | | | | | |
| 233085_s_at | hypothetical protein | FLJ22833 | AV734843 | 0.8 | 0.7 | 0.7 | 415 |
| | FLJ22833 | | | | | | |
| 229145_at | hypothetical protein | LOC119504 | AA541762 | 1.2 | 1.5 | 1.4 | 659 |
| | LOC119504 | | | | | | |
| 227550_at | hypothetical protein | LOC143381 | AW242720 | 1.2 | 1.4 | 1.4 | 222 |
| | LOC143381 | | | | | | |
| 227415_at | hypothetical protein | LOC283508 | BF109303 | 1.2 | 1.3 | 1.4 | 350 |
| | LOC283508 | | | | | | |
| 232288_at | hypothetical protein | LOC283970 | AK026209 | 1.6 | 1.5 | 1.6 | 77 |
| | LOC283970 | | | | | | |
| 226901_at | hypothetical protein | LOC284018 | AI214996 | 1.9 | 2.1 | 1.5 | 342 |
| | LOC284018 | | | | | | |
| 235482_at | hypothetical protein | LOC285002 | BE886868 | 1.6 | 1.4 | 2.0 | 132 |
| | LOC285002 | | | | | | |
| 228040_at | hypothetical protein | LOC286286 | AW294192 | 4.6 | 6.5 | 13.5 | 468 |
| | LOC286286 | | | | | | |
| 1569189_at | hypothetical protein | MGC29649 | AF289605 | 0.8 | 0.9 | 0.6 | 75 |
| | MGC29649 | | | | | | |
| 225065_x_at | hypothetical protein | MGC40157 | AI826279 | 0.8 | 0.8 | 0.7 | 237 |
| | MGC40157 | | | | | | |
| 218750_at | hypothetical protein | MGC5306 | NM_024116 | 0.9 | 0.8 | 0.7 | 239 |
| | MGC5306 | | | | | | |
| 223797_at | hypothetical protein PRO2852 | PRO2852 | AF130079 | 1.2 | 1.4 | 1.5 | 169 |
| 235756_at | IL2-UM0076-240300-056- | | AW802645 | 0.8 | 0.8 | 0.7 | 75 |
| | G02 UM0076 Homo sapiens | | | | | | |
| | cDNA, mRNA sequence. | | | | | | |
| 239842_x_at | IMAGE: 20075 Soares infant | | W18186 | 0.9 | 0.9 | 0.6 | 190 |
| | brain 1NIB Homo sapiens | | | | | | |
| | cDNA clone IMAGE: 20075, | | | | | | |
| | mRNA sequence. | | | | | | |
| 209374_s_at | immunoglobulin heavy | IGHM | BC001872 | 0.8 | 0.8 | 0.8 | 123 |
| | constant mu | | | | | | |
| 212827_at | immunoglobulin heavy | IGHM | X17115 | 0.9 | 0.7 | 0.6 | 95 |
| | constant mu | | | | | | |
| 209031_at | immunoglobulin superfamily, | IGSF4 | AL519710 | 1.3 | 1.8 | 1.6 | 921 |
| | member 4 | | | | | | |
| 201508_at | insulin-like growth factor | IGFBP4 | NM_001552 | 0.8 | 0.7 | 0.8 | 238 |
| | binding protein 4 | | | | | | |
| 226535_at | integrin, beta 6 | ITGB6 | AK026736 | 1.3 | 2.0 | 1.6 | 1574 |
| 242903_at | interferon gamma receptor 1 | | AI458949 | 0.8 | 0.7 | 0.7 | 90 |
| 224361_s_at | interleukin 17 receptor B | IL17RB | AF250309 | 0.9 | 0.7 | 0.7 | 394 |
| 229310_at | kelch repeat and BTB (POZ) | KBTBD9 | BE465475 | 1.8 | 2.0 | 1.7 | 175 |
| | domain containing 9 | | | | | | |
| 236368_at | KIAA0368 | | BF059292 | 1.7 | 2.5 | 1.5 | 142 |
| 216000_at | KIAA0484 protein | KIAA0484 | AA732995 | 0.8 | 0.8 | 0.7 | 74 |
| 231956_at | KIAA1618 | KIAA1618 | AA976354 | 1.6 | 1.8 | 2.0 | 111 |
| 238087_at | kinesin family member 2C | KIF2C | AI587389 | 1.4 | 3.2 | 1.8 | 92 |
| 1555929_s_at | laa10f11.x1 8 5 week embryo | | BM873997 | 1.2 | 1.3 | 1.5 | 230 |
| | anterior tongue 8 5 EAT | | | | | | |
| | Homo sapiens cDNA 3′, | | | | | | |
| | mRNA sequence. | | | | | | |
| 1557360_at | leucine-rich PPR-motif | LRPPRC | CA430402 | 1.6 | 2.8 | 1.8 | 103 |
| | containing | | | | | | |
| 1569003_at | likely ortholog of rat vacuole | VMP1 | AL541655 | 1.2 | 1.8 | 1.8 | 213 |
| | membrane protein 1 | | | | | | |
| 223223_at | likely ortholog of yeast ARV1 | ARV1 | AF321442 | 1.3 | 1.3 | 1.5 | 520 |
| 229554_at | lumican | LUM | AI141861 | 0.8 | 0.8 | 0.7 | 95 |
| 227438_at | lymphocyte alpha-kinase | LAK | AI760166 | 1.2 | 1.4 | 1.6 | 63 |
| 226841_at | macrophage expressed gene 1 | MPEG1 | BF590697 | 0.8 | 0.8 | 0.7 | 87 |
| 214048_at | methyl-CpG binding domain | MBD4 | AI913365 | 1.1 | 1.6 | 1.8 | 89 |
| | protein 4 | | | | | | |
| 239001_at | microsomal glutathione S- | MGST1 | AV705233 | 1.0 | 1.0 | 0.6 | 62 |
| | transferase I | | | | | | |
| 217980_s_at | mitochondrial ribosomal | MRPL16 | NM_017840 | 0.8 | 0.8 | 0.7 | 609 |
| | protein L16 | | | | | | |
| 231274_s_at | mitochondrial solute carrier | MSCP | R92925 | 1.2 | 1.3 | 1.4 | 193 |
| | protein | | | | | | |
| 1558732_at | mitogen-activated protein | MAP4K4 | AK074900 | 0.8 | 0.8 | 0.6 | 128 |
| | kinase kinase kinase kinase 4 | | | | | | |
| 223218_s_at | molecule possessing ankyrin | MAIL | AB037925 | 0.8 | 0.8 | 0.7 | 708 |
| | repeats induced by | | | | | | |
| | lipopolysaccharide (MAIL), | | | | | | |
| | homolog of mouse | | | | | | |
| 243683_at | mortality factor 4 like 2 | MORF4L2 | H43976 | 0.8 | 0.9 | 0.7 | 65 |
| 1563469_at | MRNA; cDNA | | AL832681 | 0.6 | 0.8 | 0.6 | 74 |
| | DKFZp313M0417 (from | | | | | | |
| | clone DKFZp313M0417) | | | | | | |
| 234224_at | MRNA; cDNA | | AL137541 | 0.8 | 0.7 | 0.7 | 79 |
| | DKFZp434O0919 (from clone | | | | | | |
| | DKFZp434O0919) | | | | | | |
| 227576_at | MRNA; cDNA | | AW003140 | 0.8 | 0.7 | 0.8 | 452 |
| | DKFZp686K1098 (from clone | | | | | | |
| | DKFZp686K1098) | | | | | | |
| 228217_s_at | MRNA; cDNA | | BF973374 | 1.4 | 1.3 | 1.4 | 365 |
| | DKFZp686P09209 (from | | | | | | |
| | clone DKFZp686P09209) | | | | | | |
| 210210_at | myelin protein zero-like 1 | MPZL1 | AF181660 | 1.8 | 2.4 | 1.4 | 105 |
| 233539_at | N-acyl- | NAPE-PLD | AK000801 | 1.0 | 0.8 | 0.7 | 135 |
| | phosphatidylethanolamine- | | | | | | |
| | hydrolyzing phospholipase D | | | | | | |
| 202000_at | NADH dehydrogenase | NDUFA6 | BC002772 | 0.8 | 0.9 | 0.7 | 693 |
| | (ubiquinone) 1 alpha | | | | | | |
| | subcomplex, 6, 14 kDa | | | | | | |
| 218320_s_at | neuronal protein 17.3 | P17.3 | NM_019056 | 1.0 | 1.4 | 1.8 | 993 |
| 233626_at | neuropilin 1 | NRP1 | AK024580 | 1.2 | 1.4 | 1.8 | 53 |
| 235985_at | nj45a06.x5 NCI_CGAP_Pr9 | | AI821477 | 1.3 | 1.4 | 1.6 | 115 |
| | Homo sapiens cDNA clone | | | | | | |
| | IMAGE: 995410 similar to | | | | | | |
| | contains Alu repetitive | | | | | | |
| | element; contains element | | | | | | |
| | TAR1 repetitive element;, | | | | | | |
| | mRNA sequence. | | | | | | |
| 226991_at | nuclear factor of activated T- | | AA489681 | 1.1 | 1.4 | 1.7 | 88 |
| | cells, cytoplasmic, | | | | | | |
| | calcineurin-dependent 2 | | | | | | |
| 209505_at | nuclear receptor subfamily 2, | NR2F1 | AI951185 | 1.2 | 1.5 | 1.4 | 499 |
| | group F, member 1 | | | | | | |
| 206302_s_at | nudix (nucleoside diphosphate | NUDT4 | NM_019094 | 0.9 | 0.7 | 0.7 | 955 |
| | linked moiety X)-type motif 4 | | | | | | |
| 244450_at | oc86a09.s1 | | AA741300 | 1.4 | 1.4 | 1.4 | 65 |
| | NCI_CGAP_GCBI Homo | | | | | | |
| | sapiens cDNA clone | | | | | | |
| | IMAGE: 1356568 3′ similar to | | | | | | |
| | gb: M81181 | | | | | | |
| | SODIUM/POTASSIUM- | | | | | | |
| | TRANSPORTING ATPASE | | | | | | |
| | BETA-2 (HUMAN); contains | | | | | | |
| | element PTR5 repetitive | | | | | | |
| | element;, mRNA sequence. | | | | | | |
| 238408_at | oxidation resistance 1 | OXR1 | AW086258 | 1.0 | 0.8 | 0.7 | 84 |
| 205336_at | parvalbumin | PVALB | NM_002854 | 1.4 | 1.7 | 1.9 | 319 |
| 220303_at | PDZ domain containing 2 | PDZK2 | NM_024791 | 1.3 | 1.4 | 1.5 | 95 |
| 204300_at | PET112-like (yeast) | PET112L | NM_004564 | 1.3 | 1.3 | 1.5 | 205 |
| 209504_s_at | pleckstrin homology domain | PLEKHB1 | AF081583 | 0.9 | 0.7 | 0.7 | 144 |
| | containing, family B | | | | | | |
| | (evectins) member 1 | | | | | | |
| 242922_at | pM5 protein (nomo) | PM5 | AU151198 | 1.2 | 1.4 | 1.5 | 60 |
| 236407_at | potassium voltage-gated | KCNE1 | R73518 | 1.3 | 1.5 | 1.5 | 127 |
| | channel, Isk-related family, | | | | | | |
| | member 1 | | | | | | |
| 1568706_s_at | Pp12719 mRNA, complete | | AF318328 | 1.3 | 1.6 | 1.5 | 96 |
| | cds | | | | | | |
| 1558017_s_at | PRKC, apoptosis, WTI, | PAWR | BG109597 | 1.1 | 1.1 | 1.6 | 179 |
| | regulator | | | | | | |
| 229158_at | protein kinase, lysine deficient 4 | PRKWNK4 | AW082836 | 1.2 | 1.2 | 1.5 | 859 |
| 200979_at | pyruvate dehydrogenase | PDHA1 | BF739979 | 1.3 | 1.5 | 1.5 | 650 |
| | (lipoamide) alpha 1 | | | | | | |
| 225171_at | Rho GTPase activating | ARHGAP18 | BE644830 | 1.3 | 1.2 | 1.5 | 1407 |
| | protein 18 | | | | | | |
| 221989_at | ribosomal protein L10 | RPL10 | AW057781 | 1.4 | 1.4 | 2.0 | 212 |
| 1555878_at | ribosomal protein S24 | RPS24 | AK094613 | 1.2 | 1.4 | 1.5 | 138 |
| 212030_at | RNA-binding region (RNP1, | RNPC7 | BG251218 | 1.3 | 1.5 | 1.7 | 293 |
| | RRM) containing 7 | | | | | | |
| 241996_at | RUN and FYVE domain | RUFY2 | AI669591 | 1.4 | 1.7 | 2.0 | 194 |
| | containing 2 | | | | | | |
| 215028_at | sema domain, transmembrane | SEMA6A | AB002438 | 1.2 | 1.8 | 1.5 | 63 |
| | domain (TM), and | | | | | | |
| | cytoplasmic domain, | | | | | | |
| | (semaphorin) 6A | | | | | | |
| 226492_at | sema domain, transmembrane | SEMA6D | AL036088 | 1.2 | 1.3 | 1.5 | 793 |
| | domain (TM), and | | | | | | |
| | cytoplasmic domain, | | | | | | |
| | (semaphorin) 6D | | | | | | |
| 1559263_s_at | Similar to hypothetical protein | | BG397809 | 1.1 | 1.3 | 1.7 | 96 |
| | D730019B10(LOC340152), | | | | | | |
| | mRNA | | | | | | |
| 222145_at | Similar to P1-3-kinase-related | | AK027225 | 1.6 | 1.8 | 1.7 | 64 |
| | kinase SMG-1 isoform 1; | | | | | | |
| | lambda/iota protein kinase C- | | | | | | |
| | interacting protein; | | | | | | |
| | phosphatidylinositol 3-kinase- | | | | | | |
| | related protein kinase | | | | | | |
| | (LOC390682), mRNA | | | | | | |
| 202781_s_at | skeletal muscle and kidney | SKIP | AI806031 | 1.1 | 1.4 | 1.7 | 101 |
| | enriched inositol phosphatase | | | | | | |
| 217591_at | SKI-like | SKIL | BF725121 | 1.5 | 1.9 | 1.4 | 114 |
| 220503_at | solute carrier family 13 | SLC13A1 | AF260824 | 1.3 | 1.4 | 1.5 | 501 |
| | (sodium/sulfate symporters), | | | | | | |
| | member 1 | | | | | | |
| 1559351_at | solute carrier family 16 | SLC16A9 | BI668873 | 0.8 | 0.8 | 0.6 | 138 |
| | (monocarboxylic acid | | | | | | |
| | transporters), member 9 | | | | | | |
| 206872_at | solute carrier family 17 | SLC17A1 | NM_005074 | 1.2 | 1.1 | 1.6 | 592 |
| | (sodium phosphate), member 1 | | | | | | |
| 244353_s_at | solute carrier family 2 | SLC2A12 | AI675682 | 1.7 | 1.4 | 1.8 | 125 |
| | (facilitated glucose | | | | | | |
| | transporter), member 12 | | | | | | |
| 231437_at | solute carrier family 35, | SLC35D2 | AA693722 | 1.1 | 1.2 | 1.7 | 120 |
| | member D2 | | | | | | |
| 232597_x_at | splicing factor, | SFRS2IP | AK025132 | 1.8 | 1.7 | 1.9 | 499 |
| | arginine/serine-rich 2, | | | | | | |
| | interacting protein | | | | | | |
| 232392_at | splicing factor, | SFRS3 | BE927772 | 1.4 | 1.8 | 2.1 | 565 |
| | arginine/serine-rich 3 | | | | | | |
| 237639_at | SRSR846 | | AI913600 | 1.4 | 1.6 | 1.6 | 318 |
| 204690_at | syntaxin 8 | STX8 | NM_004853 | 1.0 | 1.2 | 1.5 | 622 |
| 242512_at | te33f12.x1 | | AI382029 | 1.2 | 1.4 | 1.8 | 92 |
| | Soares_NhHMPu_S1 Homo | | | | | | |
| | sapiens cDNA clone | | | | | | |
| | IMAGE: 2088527 3′ similar to | | | | | | |
| | contains L1 t3 L1 repetitive | | | | | | |
| | element;, mRNA sequence. | | | | | | |
| 1555392_at | Testin-related protein TRG | | AY143171 | 1.2 | 1.1 | 1.7 | 74 |
| | mRNA, complete cds | | | | | | |
| 221938_x_at | thyroid hormone receptor | THRAP5 | AW262690 | 1.4 | 1.6 | 1.9 | 168 |
| | associated protein 5 | | | | | | |
| 228793_at | thyroid hormone receptor | TRIP8 | BF002296 | 1.3 | 1.4 | 2.0 | 395 |
| | interactor 8 | | | | | | |
| 232017_at | tight junction protein 2 (zona | TJP2 | AK025185 | 1.1 | 1.2 | 1.5 | 118 |
| | occludens 2) | | | | | | |
| 228971_at | Transcribed sequence with | | AI357655 | 1.0 | 1.2 | 1.6 | 704 |
| | moderate similarity to protein | | | | | | |
| | ref: NP_055301.1 (H. sapiens) | | | | | | |
| | neuronal thread protein | | | | | | |
| | [Homo sapiens] | | | | | | |
| 233518_at | Transcribed sequence with | | AU144449 | 0.8 | 0.6 | 0.7 | 74 |
| | moderate similarity to protein | | | | | | |
| | ref: NP_071431.1 (H. sapiens) | | | | | | |
| | cytokine receptor-like factor | | | | | | |
| | 2; cytokine receptor CRL2 | | | | | | |
| | precusor [Homo sapiens] | | | | | | |
| 241798_at | Transcribed sequence with | | AI339930 | 1.3 | 1.5 | 1.5 | 69 |
| | moderate similarity to protein | | | | | | |
| | sp: P39195 (H. sapiens) | | | | | | |
| | ALU8_HUMAN Alu | | | | | | |
| | subfamily SX sequence | | | | | | |
| | contamination warning entry | | | | | | |
| 243256_at | Transcribed sequence with | | AW796364 | 1.3 | 1.3 | 1.6 | 157 |
| | weak similarity to protein | | | | | | |
| | ref: NP_060265.1 (H. sapiens) | | | | | | |
| | hypothetical protein | | | | | | |
| | FLJ20378 [Homo sapiens] | | | | | | |
| 239735_at | Transcribed sequence with | | N67106 | 0.7 | 0.5 | 0.5 | 150 |
| | weak similarity to protein | | | | | | |
| | ref: NP_060312.1 (H. sapiens) | | | | | | |
| | hypothetical protein | | | | | | |
| | FLJ20489 [Homo sapiens] | | | | | | |
| 242191_at | Transcribed sequence with | | AI701905 | 1.3 | 1.4 | 1.7 | 174 |
| | weak similarity to protein | | | | | | |
| | ref: NP_060312.1 (H. sapiens) | | | | | | |
| | hypothetical protein | | | | | | |
| | FLJ20489 [Homo sapiens] | | | | | | |
| 242490_at | Transcribed sequence with | | AA564255 | 1.2 | 1.2 | 1.6 | 165 |
| | weak similarity to protein | | | | | | |
| | ref: NP_062553.1 (H. sapiens) | | | | | | |
| | hypothetical protein | | | | | | |
| | FLJ11267 [Homo sapiens] | | | | | | |
| 241897_at | Transcribed sequence with | | AA491949 | 1.3 | 1.5 | 1.9 | 492 |
| | weak similarity to protein | | | | | | |
| | ref: NP_071431.1 (H. sapiens) | | | | | | |
| | cytokine receptor-like factor | | | | | | |
| | 2; cytokine receptor CRL2 | | | | | | |
| | precusor [Homo sapiens] | | | | | | |
| 230590_at | Transcribed sequences | | BE675486 | 0.9 | 0.8 | 0.7 | 107 |
| 230733_at | Transcribed sequences | | H98113 | 0.7 | 0.6 | 0.6 | 127 |
| 230773_at | Transcribed sequences | | AA628511 | 1.1 | 1.3 | 1.6 | 131 |
| 236432_at | Transcribed sequences | | AA682425 | 0.7 | 0.6 | 0.6 | 70 |
| 237317_at | Transcribed sequences | | AW136338 | 1.0 | 0.7 | 0.7 | 79 |
| 238875_at | Transcribed sequences | | BE644953 | 1.4 | 2.3 | 2.2 | 75 |
| 239238_at | Transcribed sequences | | AI208857 | 1.4 | 1.2 | 1.4 | 113 |
| 240128_at | Transcribed sequences | | H94876 | 1.2 | 1.3 | 1.6 | 54 |
| 241837_at | Transcribed sequences | | AI289774 | 1.2 | 0.9 | 0.6 | 59 |
| 241936_x_at | Transcribed sequences | | AI654130 | 1.6 | 1.7 | 1.7 | 175 |
| 241940_at | Transcribed sequences | | BF477544 | 1.1 | 1.2 | 1.5 | 63 |
| 242299_at | Transcribed sequences | | AW274468 | 1.2 | 1.2 | 1.7 | 82 |
| 242536_at | Transcribed sequences | | AI522220 | 0.8 | 0.8 | 0.7 | 533 |
| 242579_at | Transcribed sequences | | AA935461 | 1.2 | 1.3 | 2.0 | 270 |
| 242673_at | Transcribed sequences | | AA931284 | 0.6 | 0.5 | 0.7 | 99 |
| 243591_at | Transcribed sequences | | AI887749 | 1.3 | 1.2 | 1.7 | 106 |
| 243675_at | Transcribed sequences | | BF512500 | 1.4 | 1.5 | 1.6 | 81 |
| 243933_at | Transcribed sequences | | AI096634 | 1.3 | 1.7 | 2.1 | 142 |
| 244414_at | Transcribed sequences | | AI148006 | 1.1 | 1.4 | 1.9 | 439 |
| 244674_at | Transcribed sequences | | AA936428 | 2.8 | 2.5 | 1.7 | 131 |
| 244797_at | Transcribed sequences | | AI269245 | 1.2 | 1.2 | 1.5 | 168 |
| 224566_at | trophoblast-derived | TncRNA | AI042152 | 1.3 | 1.6 | 1.5 | 1769 |
| | noncoding RNA | | | | | | |
| 204141_at | tubulin, beta polypeptide | TUBB | NM_001069 | 1.2 | 1.1 | 1.5 | 1453 |
| 202510_s_at | tumor necrosis factor, alpha- | TNFAIP2 | NM_006291 | 1.4 | 1.2 | 1.6 | 211 |
| | induced protein 2 | | | | | | |
| 232141_at | U2(RNU2) small nuclear | U2AFI | AU144161 | 1.3 | 1.4 | 1.9 | 109 |
| | RNA auxiliary factor I | | | | | | |
| 228142_at | ubiquinol-cytochrome c | HSPC051 | BE208777 | 1.5 | 1.6 | 1.7 | 177 |
| | reductase complex (7.2 kD) | | | | | | |
| 1557409_at | UI-CF-FN0-sex-p-22-0-UI.s1 | | CA313226 | 1.0 | 1.2 | 1.3 | 124 |
| | UI-CF-FN0 Homo sapiens | | | | | | |
| | cDNA clone UI-CF-FN0-aex- | | | | | | |
| | p-22-0-UI 3′, mRNA | | | | | | |
| | sequence. | | | | | | |
| 1558801_at | unnamed protein product; | | AK055769 | 1.2 | 1.6 | 1.6 | 169 |
| | Homo sapiens cDNA | | | | | | |
| | FLJ31207 fis, clone | | | | | | |
| | KIDNE2003357. | | | | | | |
| 225198_at | VAMP (vesicle-associated | VAPA | AL571942 | 1.1 | 1.4 | 1.6 | 658 |
| | membrane protein)-associated | | | | | | |
| | protein A, 33 kDa | | | | | | |
| 222303_at | v-ets erythroblastosis virus | ETS2 | AV700891 | 1.8 | 1.9 | 2.3 | 177 |
| | E26 oncogene homolog 2 | | | | | | |
| | (avian) | | | | | | |
| 235850_at | WD repeat domain 5B | WDR5B | BF434228 | 1.3 | 1.9 | 2.5 | 289 |
| 229647_at | wh65e08.x1 | | AI762401 | 1.1 | 1.2 | 1.6 | 793 |
| | NCI_CGAP_Kid11 Homo | | | | | | |
| | sapiens cDNA clone | | | | | | |
| | IMAGE: 2385638 3′ similar to | | | | | | |
| | contains Alu repetitive | | | | | | |
| | element; contains element | | | | | | |
| | MER22 repetitive element;, | | | | | | |
| | mRNA sequence. | | | | | | |
| 242406_at | w147a04.x1 NCI_CGAP_Ut1 | | AI870547 | 1.0 | 1.0 | 1.6 | 126 |
| | Homo sapiens cDNA clone | | | | | | |
| | IMAGE: 2428014 3′, mRNA | | | | | | |
| | sequence. | | | | | | |
| 224590_at | X (inactive)-specific transcript | XIST | BE644917 | 2.0 | 2.0 | 2.2 | 261 |
| 1565454_at | XAGE-4 protein | XAGE-4 | AJ318895 | 0.7 | 0.6 | 0.7 | 119 |
| 230554_at | xenobiotic/medium-chain | LOC348158 | AV696234 | 1.3 | 1.4 | 1.5 | 5210 |
| | fatty acid:CoA ligase | | | | | | |
| 238913_at | xm54d01.x1 | | AW235215 | 1.3 | 1.6 | 1.6 | 111 |
| | NCI_CGAP_GC6 Homo | | | | | | |
| | sapiens cDNA clone | | | | | | |
| | IMAGE: 2688001 3′ similar to | | | | | | |
| | contains Alu repetitive | | | | | | |
| | element; contains element | | | | | | |
| | MER28 MER28 repetitive | | | | | | |
| | element;, mRNA sequence. | | | | | | |
| 222281_s_at | xs86h03.x1 NCI_CGAP_Ut2 | | AW517716 | 1.5 | 1.6 | 1.8 | 350 |
| | Homo sapiens cDNA clone | | | | | | |
| | IMAGE: 2776565 3′ similar to | | | | | | |
| | contains Alu repetitive | | | | | | |
| | element; contains element | | | | | | |
| | MER38 repetitive element;, | | | | | | |
| | mRNA sequence. | | | | | | |
| 234033_at | yd35c06.s1 Soares fetal liver | | T71269 | 1.1 | 1.2 | 1.6 | 130 |
| | spleen INFLS Homo sapiens | | | | | | |
| | cDNA clone IMAGE: 110218 | | | | | | |
| | 3′, mRNA sequence. | | | | | | |
| 239654_at | ye62h04.s1 Soares fetal liver | | T98846 | 1.1 | 1.3 | 1.6 | 139 |
| | spleen INFLS Homo sapiens | | | | | | |
| | cDNA clone IMAGE: 122359 | | | | | | |
| | 3′, mRNA sequence. | | | | | | |
| 242241_x_at | yi33f06.s1 Soares placenta | | R66713 | 1.2 | 1.4 | 1.6 | 73 |
| | Nb2HP Homo sapiens cDNA | | | | | | |
| | clone IMAGE: 141059 3′ | | | | | | |
| | similar to contains Alu | | | | | | |
| | repetitive element; contains L1 | | | | | | |
| | repetitive element;, mRNA | | | | | | |
| | sequence. | | | | | | |
| 232216_at | YME1-like 1 (S. cerevisiae) | YME1L1 | AA828049 | 1.4 | 1.5 | 1.6 | 70 |
| 1565566_s_at | yn76g07.s1 Soares adult brain | | H21394 | 1.8 | 1.8 | 1.5 | 84 |
| | N2b5HB55Y Homo sapiens | | | | | | |
| | cDNA clone IMAGE: 174396 | | | | | | |
| | 3′ similar to contains Alu | | | | | | |
| | repetitive element;, mRNA | | | | | | |
| | sequence. | | | | | | |
| 217586_x_at | yy28g05.s1 Soares | | N35922 | 1.3 | 1.2 | 1.6 | 370 |
| | melanocyte 2NbHM Homo | | | | | | |
| | sapiens cDNA clone | | | | | | |
| | IMAGE: 272600 3′ similar to | | | | | | |
| | contains Alu repetitive | | | | | | |
| | element;, mRNA sequence. | | | | | | |
| 226163_at | zinc finger and BTB domain | ZBTB9 | AW291499 | 1.1 | 1.2 | 1.5 | 159 |
| | containing 9 | | | | | | |
| 1569312_at | zinc finger protein 146 | ZNF146 | BE383308 | 0.9 | 0.6 | 0.7 | 85 |
| 231848_x_at | zinc finger protein 207 | ZNF207 | AW192569 | 1.0 | 1.2 | 1.5 | 344 |
| 239937_at | zinc finger protein 207 | ZNF207 | AI860558 | 1.1 | 1.9 | 1.6 | 128 |
| 229279_at | zinc finger protein 432 | ZNF432 | AW235102 | 1.4 | 1.4 | 1.6 | 93 |
| 215012_at | zinc finger protein 451 | ZNF451 | AU144775 | 1.3 | 2.1 | 2.6 | 153 |
| 219741_x_at | zinc finger protein 552 | ZNF552 | NM_024762 | 1.2 | 1.3 | 1.7 | 184 |
| 230503_at | zo02d03.s1 Stratagene colon | | AA151917 | 0.7 | 0.7 | 0.7 | 159 |
| | (#937204) Homo sapiens | | | | | | |
| | cDNA clone IMAGE: 566501 | | | | | | |
| | 3′, mRNA sequence. | | | | | | |

In one embodiment, the preferred genes identified using the global analysis include, but are not limited to, ceruloplasmin (Chen et al., Biochem, Biophys Res Commun. (2001);282; 475-82), pM5/NOMO (Ju et al., Mol. Cell. Biol. (2006), 26; 654-67), colonly stimulating factor 2 receptor (Steinman et al. Annu Rev. Immunol. (1991), 9; 271-96), Hairy and enhancer of split-1 (Hes-1) (Deregowski et al. J. Biol. Chem (2006)), insulin growth factor binding protein 4 (Jehle et al, Kidney Int. (2000) 57; 1209-10), hepatocyte growth factor (hepapoietin A) (Azuma et al., J. Am. Soc. Nephrol (2001), 12; 1280-92),solute carrier family 2 (Linden et al, Am. J. Physiol Renal. Physiol. (2006) January;290(1):F205-13. Epub Aug. 9, 2005), ski-like (snoN) (Zhu et al. Mol. Cell. Biol. (2005) December;25(24):1073144).

4 Discussion

Gene expression profiling of serial renal allograft protocol biopsies was performed with the goal to identify genomic biomarkers for prediction/early diagnosis of CAN. The biomarkers are useful as molecular tools to diagnose latent CAN grade I 18 weeks and/or 12 weeks before CAN is manifest by histological parameters.

Statistical analysis of gene expression data from serial renal protocol biopsies allowed the identification of predictive/early diagnostic biomarkers of CAN I

Individual biomarker models were generated for
4.5 months before clinical/histopathol. evidence of CAN
3 months before clinical/histopathol. evidence of CAN
across timepoints and diagnosis

Biomarker variables (i.e. probe sets) are quite different at individual timepoints, here: 4.5 months and 3 months before histopathological diagnosis of CAN I

The validity of the biomarkers has to be proven by validation on new datasets.

To reveal biological processes on molecular level which are involved in the development of CAN, the analysis will focus on
temporarily expressed genes and networks, and
genes present at CAN, tracking back there expression and pathways to earlier timepoints

Equivalents

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatuses within the scope of the invention, in addition to those enumerated herein, will be apparent to those skilled in the art from the foregoing descriptions. Such modifications and variations are intended to fall within the scope of the appended claims. The present invention is to be limited only by the terms of the appended claims, along with the full scope of equivalents to which such claims are entitled.

TABLE 12
Biomarker Identification: week 12 (3 months before CAN)*

| Affymetrix | | | Fold |
|---|---|---|---|
| Probe Set ID | Description | Common | change |
| 201792_at | AE binding protein 1 | AEBP1 | 1.89 |
| 211712_s_at | annexin A9 | ANXA9 | 0.55 |
| 207367_at | ATPase, H+/K+ transporting, | ATP12A | 0.50 |
| | nongastric, alpha polypeptide | | |
| 229218_at | collagen, type I, alpha 2 | COL1A2 | 2.43 |
| 232458_at | collagen, type III, alpha 1 | | 2.79 |
| | (Ehlers-Danlos syndrome type | | |
| | IV, autosomal dominant) | | |
| 201438_at | collagen, type VI, alpha 3 | COL6A3 | 2.13 |
| 226237_at | collagen, type VIII, alpha 1 | COL8A1 | 2.17 |
| 227336_at | deltex homolog 1 (Drosophila) | DTX1 | 0.50 |
| 210165_at | deoxyribonuclease I | DNASE1 | 0.42 |
| 220625_s_at | E74-like factor 5 | ELF5 | 0.45 |
| | (ets domain transcription factor) | | |
| 221870_at | EH-domain containing 2 | EHD2 | 2.40 |
| 227353_at | epidermodysplasia | EVER2 | 3.20 |
| | verruciformis 2 | | |
| 242974_at | frizzled homolog 9 (Drosophila) | FZD9 | 2.46 |
| 211795_s_at | FYN binding protein | FYB | 2.26 |
| | (FYB-120/130) | | |
| 201744_s_at | lumican | LUM | 1.95 |
| 229554_at | lumican | LUM | 2.67 |
| 227438_at | lymphocyte alpha-kinase | LAK | 3.00 |
| 226841_at | macrophage expressed gene 1 | MPEG1 | 2.00 |
| 212999_x_at | major histocompatibility | HLA-DQB1 | 2.49 |
| | complex, class II, DQ beta 1 | | |
| 226210_s_at | maternally expressed 3 | MEG3 | 2.34 |
| 212012_at | Melanoma associated gene | D2S448 | 1.71 |
| 219666_at | membrane-spanning 4-domains, | MS4A6A | 1.94 |
| | subfamily A, member 6A | | |
| 228055_at | napsin B pseudogene | NAP1L | 1.93 |
| 214111_at | opioid binding protein/cell | OPCML | 0.40 |
| | adhesion molecule-like | | |
| 205267_at | POU domain, class 2, | POU2AF1 | 4.04 |
| | associating factor 1 | | |
| 216834_at | regulator of G-protein | RGS1 | 2.69 |
| | signalling 1 | | |
| 218870_at | Rho GTPase activating | ARHGAP15 | 2.52 |
| | protein 15 | | |
| 209374_s_at | immunoglobulin heavy | IGHM | 8.84 |
| | constant mu | | |
| 203083_at | thrombospondin 2 | THBS2 | 2.23 |
| 209960_at | hepatocyte growth factor (HGF). | HGF | 2.00 |
| 202664_at | Wiskott-Aldrich syndrome | WASPIP | 1.96 |
| | protein interacting protein | | |

*Probe sets of biomarker without functionally non-annotated probe sets omitted

TABLE 13
Week 12 (3 months prior to histological diagnosis of CAN):
Large overrepresentation of immune related genes

| Affymetrix ID | Gene Name | FC | T test |
|---|---|---|---|
| 213539_at | CD3D antigen, delta polypeptide | 2.5 | 1.3E−02 |
| | (TiT3 complex) | | |
| 210031_at | CD3Z antigen, zeta polypeptide | 2.1 | 1.4E−02 |
| | (TiT3 complex) | | |
| 212063_at | CD44 antigen (homing function and | 2.1 | 8.2E−02 |
| | Indian blood group system) | | |
| 204118_at | CD48 antigen | 1.7 | 6.1E−02 |
| | (B-cell membrane protein) | | |
| 213958_at | CD6 antigen | 2.3 | 2.8E−02 |
| 206978_at | chemokine (C-C motif) receptor 2 | 2.3 | 6.4E−03 |
| 206337_at | chemokine (C-C motif) receptor 7 | 2.0 | 7.4E−02 |
| 205898_at | chemokine (C—X3—C motif) | 2.0 | 4.4E−03 |
| | receptor 1 | | |
| 217028_at | chemokine (C—X—C motif) | 2.1 | 1.1E−01 |
| | receptor 4 | | |
| 224733_at | chemokine-like factor super family 3 | 1.4 | 2.9E−01 |
| 224998_at | chemokine-like factor super family 4 | 0.6 | 4.3E−03 |
| 211339_s_at | IL2-inducible T-cell kinase | 2.1 | 2.6E−02 |
| 232024_at | immunity associated protein 2 | 1.7 | 9.6E−02 |
| 211430_s_at | immunoglobulin heavy constant | 8.5 | 2.9E−02 |
| | gamma 3 (G3m marker) | | |
| 209374_s_at | immunoglobulin heavy constant mu | 25.6 | 1.2E−02 |
| 212827_at | immunoglobulin heavy constant mu | 1.6 | 1.2E−01 |
| 212592_at | immunoglobulin J polypeptide, linker | 9.9 | 1.9E−02 |
| | protein for immunoglobulin al [symbol] | | |
| 214677_x_at | immunoglobulin lambda joining 3 | 10.7 | 3.5E−02 |
| 215121_x_at | immunoglobulin lambda locus | 14.8 | 5.4E−02 |
| 209031_at | immunoglobulin superfamily, | 0.5 | 1.2E−02 |
| | member 4 | | |
| 226818_at | macrophage expressed gene 1 | 2.4 | 2.8E−02 |
| 226841_at | macrophage expressed gene 1 | 2.7 | 1.1E−04 |
| 211654_x_at | major histocompatibility complex, | 1.8 | 5.9E−02 |
| | class II, DQ beta 1 | | |
| 212999_x_at | major histocompatibility complex, | 2.9 | 6.3E−03 |
| | class II, DQ beta 1 | | |
| 209312_x_at | major histocompatibility complex, | 1.7 | 1.0E−01 |
| | class II, DR beta 3 | | |
| 204670_x_at | major histocompatibility complex, | 1.6 | 5.0E−03 |
| | class II, DR beta 4 | | |
| 208306_x_at | major histocompatibility complex, | 1.6 | 9.6E−03 |
| | class II, DR beta 4 | | |
| 202687_s_at | tumor necrosis factor | 1.7 | 2.0E−01 |
| | (ligand) superfamily, member 10 | | |
| 214329_x_at | tumor necrosis factor | 1.5 | 1.5E−01 |
| | (ligand) superfamily, member 10 | | |
| 204781_s_at | tumor necrosis factor | 1.7 | 2.7E−02 |
| | receptor superfamily, member 6 | | |
| 202510_s_at | tumor necrosis factor, | 1.6 | 8.4E−02 |
| | alpha-induced protein 2 | | |
| 202644_s_at | tumor necrosis factor, | 1.6 | 8.2E−02 |
| | alpha-induced protein 3 | | |
| 206026_s_at | tumor necrosis factor, | 2.8 | 4.4E−02 |
| | alpha-induced protein 6 | | |
| 210260_s_at | tumor necrosis factor, | 1.7 | 3.7E−03 |
| | alpha-induced protein 8 | | |

[symbol]

indicates data missing or illegible when filed

TABLE 14
Week 12 (3 months prior to histological diagnosis of CAN):
Large overrepresentation of ECM related genes

| Affymetrix ID | Gene Name | FC | T test |
|---|---|---|---|
| 1556499_s_at | collagen, type I, alpha 1 | 1.5 | 8.0E−02 |
| 202403_s_at | collagen, type I, alpha 2 | 1.7 | 3.2E−02 |
| 202404_s_at | collagen, type I, alpha 2 | 2.0 | 3.1E−02 |
| 229218_at | collagen, type I, alpha 2 | 2.3 | 6.4E−03 |
| 201852_x_at | collagen, type III, alpha 1 | 2.1 | 1.7E−02 |
| | (Ehlers-Danlos syndrome type IV, | | |
| | autos [symbol] | | |
| 215076_s_at | collagen, type III, alpha 1 | 1.7 | 1.4E−02 |
| | (Ehlers-Danlos syndrome type IV | | |
| | autos [symbol] | | |
| 212488_at | collagen, type V, alpha 1 | 2.0 | 1.5E−02 |
| 212489_at | collagen, type V, alpha 1 | 1.6 | 5.2E−02 |
| 209156_s_at | collagen, type VI, alpha 2 | 2.2 | 6.9E−02 |
| 201438_at | collagen, type VI, alpha 3 | 2.3 | 2.6E−03 |
| 226237_at | collagen, type VIII, alpha 1 | 2.8 | 2.1E−02 |
| 212865_s_at | collagen, type XIV, alpha 1 (undulin) | 1.7 | 9.9E−03 |
| 204345_at | collagen, type XVI, alpha 1 | 1.6 | 1.1E−02 |
| 201893_x_at | decorin | 1.6 | 4.2E−02 |
| 209335_at | decorin | 1.5 | 3.8E−02 |
| 211813_x_at | decorin | 1.5 | 2.1E−01 |
| 211896_s_at | decorin | 1.7 | 3.0E−02 |
| 210495_x_at | fibronectin 1 | 1.5 | 2.1E−01 |
| 211719_x_at | fibronectin 1 | 1.5 | 2.4E−01 |
| 212464_s_at | fibronectin 1 | 1.5 | 2.2E−01 |
| 218255_s_at | fibrosin 1 | 0.6 | 1.2E−03 |
| 202995_s_at | fibulin 1 | 2.0 | 1.6E−02 |
| 202994_s_at | fibulin 1 | 1.6 | 8.8E−02 |
| 201744_s_at | lumican | 1.9 | 2.2E−02 |
| 229554_at | lumican | 2.9 | 8.4E−04 |
| 204259_at | matrix metalloproteinase 7 | 1.8 | 2.3E−01 |
| | (matrilysin, uterine) | | |

[symbol]

indicates data missing or illegible when filed

TABLE 15
Overview for “Global Analysis”.

| | |
|---|---|
| Intention: | Identification of biomarker model across timepoints and |
| | diagnosis |
| Samples: | 33 N samples from non-progressors (“N”) |
| | 8 pre-CAN, week 6 (“week 06 pre-CAN”) |
| | 8 pre-CAN, week12 (“week 12 pre-CAN”) |
| | 18 CAN grd. I (week 6, 12 and 24) (“CAN”) |
| | total: 67 samples |
| Normalization: | each group to median of N samples, by batch |
| Filter: | Coefficient of Variation: small (<20% in group N) |
| | Raw expression values >100 in >25% of all samples) |
| Analysis: | SIMCA (OSC, i.e partial least square with |
| | orthogonal signal correction) |

TABLE 16
Excerpt of genes from the global analysis

| | | | Fold change |
|---|---|---|---|
| Affymetrix ID | Gene Name | Role | trend |
| 223679_at | catenin (cadherin-associated protein), beta 1, | Wnt pathway; EMT | [symbol] |
| | 88 kDa | | |
| 228143_at | ceruloplasmin (ferroxidase) | copper carrier; elevated in serum in | [symbol] |
| | | nephrotic syndrome | |
| 225288_at | collagen, type XXVII, alpha 1 | ECM | [symbol] |
| 1556820_a_at | deleted in lymphocytic leukemia, 2 | | [symbol] |
| 210165_at | deoxyribonuclease I | tubular damage | [symbol] |
| 227353_at | epidermodysplasia verruciformis 2 | | [symbol] |
| 203394_s_at | hairy and enhancer of split 1, (Drosophila) | Notch signaling; T cell; regulation of | [symbol] |
| | | prostaglandin synthase | |
| 209960_at | HGF (AA 1-728) | antagonizes TGFbeta; ameliorates | [symbol] |
| | | interstitial inflammation; inhibits EMT | |
| 212827_at | IgM heavy chain complete sequence. | immune response | [symbol] |
| 242903_at | interferon gamma receptor 1 | | [symbol] |
| 227438_at | lymphocyte alpha-kinase | maintenance of epithelial polarity | [symbol] |
| 226841_at | macrophage expressed gene 1 | | [symbol] |
| 226991_at | nuclear factor of activated T-cells, cytoplasmic, | potential metabolic sensor for the | [symbol] |
| | calcineurin-dependent 2 | arterial smooth muscle response to | |
| | | high glucose; immune response | |
| 206302_s_at | nudix (nucleoside diphosphate linked moiety X)- | pyrophosphate hydrolase | [symbol] |
| | type motif 4 | | |
| 1558017_s_at | Prostate apoptosis response-4 protein | interacts with WT1; apoptosis | [symbol] |
| 217591_at | SKI-like | TGFbeta pathway; interacts with | [symbol] |
| | | Smad3 | |
| 221938_x_at | thyroid hormone receptor associated protein 5 | | [symbol] |
| 228793_at | thyroid hormone receptor interactor 8 | | [symbol] |
| 224566_at | trophoblast MHC class II suppressor | non-coding RNA; suppresses MHC | [symbol] |
| | | class expression | |
| 202510_s_at | tumor necrosis factor, alpha-induced protein 2 | | [symbol] |

1. A method for predicting the onset of a rejection of a transplanted organ in a subject, comprising the steps of:
(a) obtaining a post-transplantation sample from the subject;
(b) determining the magnitude of gene expression in the post-transplantation sample of a combination of a plurality of genes selected from the group consisting of the genes set forth in Table 4, Table 5, Table 6, Table 7, and Table 8 in combination with a predictive model selected from the group consisting of a PLDSA model and an OPLS model;
(c) comparing the magnitude of gene expression of the combination of the plurality of genes in the post-transplantation sample with the magnitude of gene expression of the same combination of the plurality of genes in a control sample; and
(d) determining whether the magnitude of gene expression of the combination of the plurality of genes is up-regulated or down-regulated relative to the control sample, wherein up-regulation or down-regulation of the magnitude of expression of the combination of the plurality of genes indicates that the subject is likely to experience transplant rejection, thereby predicting the onset of rejection of the transplanted organ in the subject.

2. The method of claim 1, wherein the post-translation sample comprises cells obtained from the subject.

3. The method of claim 1, wherein the post-translation sample is selected from the group consisting of: a graft biopsy; blood; serum; and urine.

4. The method of claim 1, wherein the rejection is chronic/sclerosing allograph nephropathy.

5. The method of claim 1, wherein the magnitude of expression in the post-transplantation sample differs from the magnitude of expression in the control sample by a factor of at least about 1.5.

6. The method of claim 1, wherein the magnitude of expression in the post-transplantation sample differs from the magnitude of expression in the control sample by a factor of at least about 2.

7. A method for predicting the onset of a rejection of a transplanted organ in a subject, comprising the steps of:
(a) obtaining a post-transplantation sample from the subject;
(b) determining the gene expression pattern in the post-transplantation sample of a combination of a plurality of genes selected from the group consisting of the genes set forth in Table 4, Table 5, Table 6, Table 7, and Table 8 in combination with a predictive model selected from the group consisting of a PLDSA model and an OPLS model; and
(c) comparing the gene expression pattern of the combination of the plurality of genes in the post-transplantation sample with the gene expression pattern of the same combination of the plurality of genes in a control sample, wherein a similarity in the gene expression pattern of the combination of the plurality of genes in the post-transplantation sample compared to the gene expression pattern of the same combination of the plurality of genes in a control sample gene expression pattern indicates indicates that the subject is likely to experience transplant rejection, thereby predicting the onset of rejection of the transplanted organ in the subject.

8. The method of claim 7, wherein the post-transplantation sample comprises cells obtained from the subject.

9. The method of claim 7, wherein the post-transplantation sample is selected from the group consisting of: a graft biopsy; blood; serum; and urine.

10. The method of claim 7, wherein the rejection is chronic/sclerosing allograft nephropathy.

11. A method of monitoring transplant rejection in a subject, comprising the steps of:
(a) taking as a first baseline value the magnitude of gene expression of a combination of a plurality of genes in a sample obtained from a transplanted subject who is known not to develop rejection;
(b) taking as a second value the magnitude of gene expression of the same combination of a the plurality of genes in a sample obtained from a the transplanted subject post-transplantation; and
(c) comparing the first baseline value with the second value, wherein a first baseline value lower or higher than the second value predicts that the transplanted subject is at risk of developing rejection, wherein the combination of the plurality of genes are selected from the group consisting of the genes set forth in Table 4, Table 5, Table 6, Table 7, and Table 8 in combination with a predictive model selected from the group consisting of a PLDSA model and an OPLS model, thereby monitoring transplant rejection in the subject.

12. A method of monitoring transplant rejection in a subject, comprising the steps of:
(a) taking as a first value a pattern of gene expression corresponding to a combination of a plurality of genes from a sample obtained from a donor subject at the day of transplantation;
(b) taking as a second value a pattern of gene expression corresponding to the combination of the plurality of genes from a sample obtained from a recipient subject post-transplantation; and
(c) comparing the first value with the second value, wherein a first value lower or higher than the second value predicts that the recipient subject is at risk of developing rejection; wherein the a combination of the plurality of genes selected from the group consisting of the genes set forth in Table 4, Table 5, Table 6, Table 7, and Table 8 in combination with a predictive model selected from the group consisting of a PLDSA model and an OPLS model, thereby monitoring transplant rejection in the subject.

13. A method for monitoring modifying transplant rejection treatment in a subject at risk thereof, comprising the steps of:
(a) obtaining a pre-administration sample or samples from a transplanted subject prior to administration of a rejection inhibiting agent;
(b) detecting the pattern of gene expression of a plurality of genes in the pre-administration sample or samples; and
(c) obtaining a post-administration sample or samples from the transplanted subject;
(d) detecting the pattern of gene expression of a the plurality of genes in the post-administration sample or samples;
(e) comparing the pattern of gene expression of the plurality of genes in the pre-administration sample with the pattern of gene expression in the post-administration sample or samples; and
(f) adjusting the agent accordingly, wherein the plurality of genes are selected from the group consisting of the genes Table 4, Table 5, Table 6, Table 7, and Table 8 in combination with a predictive model selected from the group consisting of a PLDSA model and an OPLS model, thereby modifying transplant refection treatment.

14. A method for preventing, inhibiting, reducing or treating transplant rejection in a subject in need of such treatment comprising administering to the subject a compound that modulates the synthesis, expression or activity of one or more genes or gene products encoded thereby, said genes being selected from the group consisting of the genes set forth in Table 4, Table 5, Table 6, Table 7, and Table 8 in combination with a predictive model selected from the group consisting of a PLDSA model and an OPLS model, such that at least one symptom of rejection is ameliorated.

15. (canceled)

16. The method according to claim 1, wherein the transplanted subject is a kidney transplanted subject.

17. The method according to of claim 1, wherein the magnitude of gene expression is assessed by detecting the presence of a protein encoded by the combination of the plurality of genes.

18. The method of claim 17, wherein the presence of the protein is detected using a reagent which specifically binds to the protein.

19. The method of claim 12, wherein the pattern of gene expression is detected by techniques selected from the group consisting of Northern blot analysis, reverse transcription PCR and real time quantitative PCR.

20-24. (canceled)
